(19) United States
(12) Patent Application Publication
(10) Pub. No.: US 2012/0202756 A1
(43) Pub. Date: Aug. 9, 2012

(54) USE OF PRODRUGS TO AVOID GI MEDIATED ADVERSE EVENTS

(76) Inventors: Richard Franklin, Basingstoke (GB); Martin Quibell, (US); Robert G. Tyson, (US); Bernard T. Golding, (US)

(21) Appl. No.: 13/253,677

(22) Filed: Oct. 5, 2011

Related U.S. Application Data
(63) Continuation-in-part of application No. 12/753,042, filed on Apr. 1, 2010.
(60) Provisional application No. 61/211,831, filed on Apr. 2, 2009.
(60) Provisional application No. 61/227,716, filed on Jul. 22, 2009.

(30) Foreign Application Priority Data
Oct. 5, 2010 (GB) GB1016752.6
Jul. 4, 2011 (GB) GB1111378.4
Jul. 4, 2011 (GB) GB1111382.6

(51) Int. Cl. A61K 31/7056; C07H 15/14; C07C 229/22; C07C 229/60; C07D 211/46; C07D 211/22; C07D 417/12; C07D 403/12; A61K 31/4164; A61K 31/225; A61K 31/24; A61K 31/445; A61K 31/554; A61K 31/506; A61P 31/04; A61P 9/06; A61P 25/28; A61P 19/02; A61P 25/24; A61P 35/00; A61P 19/10; A61P 31/12; A61P 3/10; C07D 233/94

(52) U.S. Cl. 514/24; 548/328.5; 536/16.8; 560/171; 560/44; 546/242; 546/206; 544/49; 544/324; 514/398; 514/548; 514/533; 514/315; 514/319; 514/211.09; 514/275

(57) ABSTRACT

The present invention relates to prodrugs of a wide variety of drugs and pharmaceutical compositions containing such prodrugs. Methods for minimizing locally mediated (from within the gut lumen) adverse gastrointestinal events associated with the underivatised drug and increasing the sustainment of plasma drug levels with the aforementioned prodrugs are also provided. Thus, the present invention relates to the use of prodrugs of a wide diversity of drugs (other than opioids) to transiently inactivate them and so reduce directly, locally mediated adverse gastrointestinal (GI) side-effects normally evident after administration of the parent compound. Additionally, such prodrugs may confer improved pharmacokinetics.

### CROSS-REFERENCE TO RELATED APPLICATIONS

This application claims benefit under 35 U.S.C. §120 and is a continuation-in-part of U.S. application Ser. No. 12/753,042 filed on Apr. 1, 2010, which claims priority under 35 U.S.C. §119(e) U.S. Provisional Application No. 61/211,831, filed on Apr. 2, 2009 and U.S. Provisional Application No. 61/227,716, filed on Jul. 22, 2009. This application als claims benefit under 35 U.S.C. §119(e) to Great Britian Application Nos. GB1016752.6, filed on Oct. 5, 2010; GB1111382.6, filed on Jul. 4, 2011, and GB1111378.4 filed on Jul. 4, 2011. This application is related to co-pending International Application No. PCT/GB2011/051911, filed Oct. 5, 2011. Each of the applications above are hereby incorporated by references in their entirety.

### FIELD OF THE INVENTION

The present invention relates to the use of prodrugs of a wide diversity of drugs (other than opioids) to transiently inactivate them and so reduce directly, locally mediated adverse gastrointestinal (GI) side-effects normally evident after administration of the parent compound. Additionally, such prodrugs may confer improved pharmacokinetics.

### BACKGROUND OF THE INVENTION

Many, inherently valuable, older “gold standard” medications have associated with them adverse GI events which can limit their utility. Patients may be reluctant to continue with medication which causes profound constipation, diarrhoea or nausea and/or vomiting leading to so-called serious “patient non-compliance” and inadequate treatment. Such side-effects may also limit the maximum dose which can be used, denying the possibility of achieving an optimal dosage. In some situations these GI side-effects may potentially present a serious medical risk as for example with the use of non-steroidal anti-inflammatory agents which can cause gastric or duodenal ulceration and potentially perforation and haemorrhage. Similarly serious oesophageal ulcers may occur with the use bisphosphonates especially in patients with hiatal hernias.

In many instances these adverse GI effects are the result, in part or wholly, of direct drug action from within the GI tract i.e. they are locally mediated from within the gut lumen. Sometimes the systemic pharmacological target receptor may also be present in the gut and interactions at this level may cause unwanted local effects. Alternatively there may be a direct irritant effect of the drug on the gut or the drug may, in some cases, result in a disturbance of normal gut flora.

An example of the latter is to be found in the use of many oral antibiotics. Antibiotic associated diarrhoea occurs in about 5-30% of patients either during early antibiotic therapy or up to 2 months after the end of treatment. While almost all antibiotics, particularly those that acting on anaerobes, can cause diarrhoea, this is higher with aminopenicillins, cephalosporins and clindamycin and lincomycin. A prominent influencing factor in such effects is the extent of absorption and also entero hepatic circulation which may lead to drug reaching the lower gut whereupon it can act locally on the gut microflora in the large bowel. This disruption in the normal gut microfloral population can lead to subsequent dominance of Clostridium difficile, and pronounced diarrhoea. The resultant water loss and dehydration can be dangerous especially for the frail, the elderly and otherwise compromised patient groups. Cessation of treatment with the oral antibiotic usually results in the symptoms remitting but in some cases chronic pseudomembraneous colitis may become established. The cephalosporins are particularly valuable broad spectrum antibiotics but are associated with the development of C. difficile colonisation of the gut due to alteration of the normal gut microfloral balance. Furthermore some of the newer cephalosporins such as ceftobiprole and ceftaroline which are effective against methicillin resistant Staphylococcus aureus (MRSA) are poorly orally absorbed whether free or as their respective prodrugs, ceftobiprole medocaril and ceftaroline fosamil (El Sohl AA (2009) Expert Opinion Pharmacother. 10 1675-1686). There is a real need to improve the oral absorption of these compounds to be of greater value. The use of the valuable broad spectrum fluoroquinolones has been much restricted due to their side-effects which include not only overgrowth of C difficile but in some cases methicillin resistant Staphylococcus aureus in the gut due to disruption of the normal gut microfloral population (LeBlanc L et al (2006) Emerg. Infect. Dis. 12 1398-13405). Overcoming this risk would be hugely clinically beneficial.

Another class of drugs blighted by significant GI side effects are the sodium channel blocking class 1 anti-arrhythmics. Compounds such as propafenone are associated with marked nausea and vomiting which may be ascribed to their direct local anaesthetic activity in inhibiting slow wave movement of the stomach which may lead to gastric stasis and emesis.

The adverse GI effects of systemic glucosylceramide synthase inhibitors used for Gaucher disease, such as miglustat, result from an action on gut disaccharidases (Giraldo P et al (2009) Haematologica 94 1771-1775), which causes profound diarrhoea. According to the FDA label for miglustat, up to 89% of patients treated with miglusat suffer from diarrhoea. Management of these side-effects involves restriction to a low carbohydrate diet and the use of antidiarrhoeal drugs such as loperamide. However these measures may not be fully effective.

Acetyl choline esterase inhibitors such as donepezil used in the treatment of Alzheimer's disease are associated with marked nausea, vomiting and diarrhoea. The incidence of nausea after a daily dose of 10 mg was ˜24% while diarrhoea was seen in some 17% of patients after the same dosage (Geldmecher DS (2004) 4, 5-16). Both donepezil and rivastigmine are reputed to be better tolerated when given by the transdermal route avoiding direct instillation into the gut (Terahara T et al (2007) WO2007/129712, Sadowsky C H (2010) Int J Clin Pract. 64, 188-93). This tends to suggest that their emetic effects may be brought about directly in the gut via a local cholinergic mechanism. Such is the problem of nausea and vomiting associated with oral administration of donepezil that slow dose titration is needed to minimize the risk of patient discontinuation (FDA Label). However even with this approach, optimally effective doses may never be reached in some patients due to intolerance to the adverse GI side-effects.

Non-steroidal anti-inflammatory drugs may lead to stomach ulcers and other serious complications such as GI bleeding or perforation. One study estimates that these complications cause >100,000 hospital admissions and 16,500 deaths each year in the United States (Wolf M M et al (1999) N Engl J Med 340-1888-89). While these effects are generally thought to be, in large part, the result of systemic inhibition of prostaglandin synthetase, there is also considered to be an importantcontribution from direct local irritancy of these acidic molecules within the stomach and upper GI tract (Fiorucci S et al (2001) Digest Liver Dis 33 (Suppl 2) S35-43). Hence the use of enteric coated formulations in an attempt to mitigate this upper GI irritant effect. Additionally patients may be given either H2 antagonists or proton pump inhibitors or misoprostol in an attempt to ameliorate the effects of gastric acid production on the ulceration. However, these measures are not always effective.

The use of serotonin re-uptake inhibitors, SSRI's, for the treatment of depression, is associated with marked gastrointestinal side effects such as nausea, diarrhoea and constipation. Up to 25% of treated patients may be so affected which can limit the use of these compounds. The expression of the serotonin re-uptake transporter (SERT) in the gastrointestinal mucosa and the known role of serotonin in the propulsive actions of the gut suggests that these effects are the result of a direct action of these compounds within the lumen (Gerson MD(2000) Gastroenterol 16 113-120).

Use of oral anticancer agents is beset by problems of diarrhoea and vomiting involving a variety of different mechanisms. Chemotherapeutic agents cause nausea and vomiting by stimulating serotonin release from entero-chromaffin cells within the gut which activates the vagus (Fortun P & Hawkey C J (2007) Medicine 35 210-215). The use of 5-HT3 antagonists such as ondansetron and granesetron to counter these effects may be only partially successful. Newer tyrosine kinase anti-cancer agents such as erlotinib (Tarceva) targeting epidermal growth factor receptor (EGFR) can in result in diarrhoea in 50-60% of patients treated. The incidence of diarrhoea correlated with dose and not with plasma concentrations suggesting a direct effect of erlotinib on the gastrointestinal tract. EGFR is widely expressed in the normal colonic mucosa which regulates both chloride secretion and sodium absorption by colonocytes. Thus EFGR inhibition can subsequently lead to secretory diarrhoea (Asnacios A et al (2009) Eur J Cancer 45 Suppl 1:332-42). Another tyrosine kinase inhibitor, this time of vascular endothelial growth factor receptor (VGFR), pazopanib results in some 50% of patients being affected with diarrhoea and may exert this effect by a similar mechanism.

Bisphosphonates, used in the treatment of osteoporosis, represent another class of drug associated with unwanted GI effects. Between 25 and 50% of patients experience serious gastrointestinal side-effects including ulceration of the oesophagus. To minimize the risk of upper GI irritation patients are instructed to ensure tablets are taken on arising (not at bedtime), that they should not be taken with liquids other than water, and that patients should wait at least 30 minutes after dosing before eating, drinking, or taking any other medications. The action of this class of compounds on the gut, including the oesophagus, has been unequivocally proven to be due to a direct irritant action on the mucosa. Perfusion of the dog oesophagus with alendronate caused marked oesophageal damage (Peter C P et al (1998) Dig Dis Sci 43 1998-2002) while other studies on the rat gastric mucosa likewise showed injury after direct application (Kanatsu K et al (2004) J Gastroenterol Hepatol 19, 512-20).

Yet a further example of adverse GI events likely to be locally mediated, is seen with many antiviral drugs. For example amprenavir has an incidence of nausea, vomiting and diarrhoea of 74, 34 and 39% respectively. Stavudine has an incidence of diarrhoea of 44%, with similar levels of nausea and vomiting (39-44%). Didananosine has an incidence of diarrhoea of 19-28%. Although there has been no systematic investigation into the mechanism underlying these adverse GI events it is relevant to note that enteric coated capsules of didanosine are associated with an improved GI tolerability (Moreno S et al (2007) Drugs 67 1441-1462) implying these GI adverse events are effected by direct local drug action within the stomach.

Another class of therapeutic agent associated with marked GI side effects include the biguanide anti-diabetic agents typified by metformin. In a study using 141 patients receiving metformin, the incidence of diarrhoea was 53% compared to 11.7% in those receiving placebo, vomiting was recorded in 25.5% of patients on therapy compared to 8.3% receiving placebo. Diarrhoea led to a discontinuation of the medication in 6% of these patients (Prescribing information on Glucophage® and Glucophage XR® Bristol-Myers Squibb). Again no detailed pharmacology studies have been reported investigating the mechanism of metformin induced adverse GI effects but much circumstantial evidence, including the lesser GI effects associated a sustained release formulation, suggest a role for direct action from within the lumen.

Yet another compound with which GI side-effects are evident is acetazolamide. These can include taste alteration and gastrointestinal disturbances such as nausea, vomiting and diarrhoea and may be the result of locally mediated inhibition of carbonic anhydrase in the gut mucosa.

As indicated above, the consequences of locally mediated GI adverse events are wide ranging. Minimally they can result in poor or erratic patient compliance leading to under medication of the patients. Under medication may also result from drug loss due to vomiting or to incomplete absorption due to intestinal hurry/diarrhoea. Amongst the more serious outcomes, the use of NSAIDS may result in life-threatening major gastric haemorrhage while antibiotic use in elderly patients succumbing to C. difficile infection may result in dangerous dehydration.

None of the adjunctary medication or other strategies for dealing with these GI adverse events are entirely effective. There thus remains a real need in the treatment of a wide range of medical conditions, for drug products which retain all the inherent pharmacological advantages of the parent molecule but which avoid or minimize their principal limitations, namely induction of adverse GI side effects.

### SUMMARY OF THE INVENTION

According to a first aspect of the present invention, there is provided a prodrug for treating a systemic disorder, the prodrug having a structure of Formula (I) or a pharmaceutically acceptable salt thereof:

D-L-R (I)

wherein:

D—is a non-opioid drug having a free hydroxyl group, a free amine group or an enolisable carbonyl group, the drug being for use in treating said systemic disorder;

—R is an amino acid residue containing from 2 to 20 carbon atoms or a peptide formed from 2 to 10 independently selected amino acids each containing from 2 to 20 carbon atoms; or —R is an amino amide residue containing from 2 to 20 carbon atoms and terminating with a —CONR^{a}R^{b} group, or —R is a peptide formed from 2 to 9 independently selected amino acids each containing from 2 to 20 carbon atoms and terminating with an amino amide residue containing from 2 to 20 carbon atoms, the amino amide residue terminating with a —CONR^{a}R^{b} group;

R^{a} and R^{b} when present are each independently selected from the group consisting of: H, C₁₋₆ alkyl, —(CH₂)ᵣ—C₃₋₆ cycloalkyl, phenyl and benzyl, or wherein R^{a} and R^{b} together with the nitrogen atom to which they are attached form a ring containing 3, 4, 5 or 6 carbon atoms; wherein each of the R^{a} and R^{b} groups may be unsubstituted or substituted with 1 or 2 substituent groups independently selected at each occurrence from the group consisting of: F, CI, CN and OH; r is an integer of 0 or 1;

-L- is

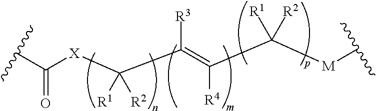

R¹ and R² are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷; or together with the atom to which they are bonded, R¹ and R² may form a carbonyl, an ethylene or a C₃₋₆ cycloalkyl; and

R³ and R⁴ are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷;

R⁵ and R⁶ are each independently selected from the group consisting of: H, C₁₋₆ alkyl, C₁₋₆ haloalkyl, C₃₋₈ cycloalkyl and phenyl;

R⁷ is selected from the group consisting of: hydroxyl, C₁₋₆ alkyl, C₁₋₆ alkoxy, C₃₋₈ cycloalkyl and phenyl;

X is selected from the group consisting of: a bond, —O—, —NH—, and a saturated or unsaturated ring having from 3 to 6 carbon atoms in the ring;

M is selected from the group consisting of:

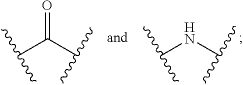

n and p are each independently an integer of 0-16, provided that the sum of n and p is an integer of 0-16;

m is an integer of 0-2; and

q is an integer of 0-3.

The terminal portion of the amino acid residue or peptide (—R) may be in the form of the free acid i.e. terminating in a —COOH group or may be in a masked (protected) form such as in the form of a carboxylate ester or carboxamide. Sometimes, the amino acid or peptide residue terminates with an amino group.

In an embodiment, the amino acid residue or peptide (—R) terminates with a carboxylic acid group —COOH or an amino group —NH₂. In another embodiment, the residue terminates with a carboxamide group CONR^{a}R^{b}. In an alternate embodiment, the residue terminates with a carboxylate ester COOR^{a}.

According to a second aspect of the present invention, there is provided a prodrug for treating a systemic disorder, the prodrug having a structure of Formula (II) or a pharmaceutically acceptable salt thereof:

D-L-R (II)

wherein:

D- is a non-opioid drug having a free hydroxyl group, a free amine group or an enolisable carbonyl group, the drug being for use in treating said systemic disorder;

—R is —OR^{a} or —NR^{a}R^{b};

R^{a} and R^{b} are each independently selected from the group consisting of: H, C₁₋₆ alkyl, —(CH₂)ᵣ—C₃₋₆ cycloalkyl, phenyl and benzyl, or wherein R^{a} and R^{b} together with the nitrogen atom to which they are attached form a ring containing 3, 4, 5 or 6 carbon atoms; wherein each of the R^{a} and R^{b} groups may be unsubstituted or substituted with 1 or 2 substituent groups independently selected at each occurrence from the group consisting of: F, CI, CN and OH; r is an integer of 0 or 1;

-L- is

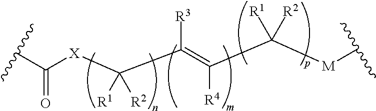

R¹ and R² are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷; or together with the atom to which they are bonded, R¹ and R² may form a carbonyl, an ethylene or a C₃₋₆ cycloalkyl; and

R³ and R⁴ are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷;

R⁵ and R⁶ are each independently selected from the group consisting of: H, C₁₋₆ alkyl, C₁₋₆ haloalkyl, C₃₋₈ cycloalkyl and phenyl;

R⁷ is selected from the group consisting of: hydroxyl, C₁₋₆ alkyl, C₁₋₆ alkoxy, C₃₋₈ cycloalkyl and phenyl;

X is selected from the group consisting of: a bond, —O—, —NH—, and a saturated or unsaturated ring having from 3 to 6 carbon atoms in the ring;

M is selected from the group consisting of:

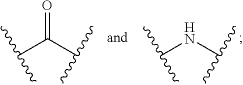

n and p are each independently an integer of 0-16, provided that the sum of n and p is an integer of 0-16;

m is an integer of 0-2; and

q is an integer of 0-3.

The non-opioid drug normally exhibit adverse gastrointestinal effects when administered alone (i.e. without being covalently bonded to -L-R). However, the prodrug of Formula I exhibits reduced adverse gastrointestinal effects compared to the adverse gastrointestinal effects of the non-opioid drug when administered alone (i.e. without being covalently bound to -L-R).

According to a third aspect of the present invention, there is provided a prodrug having a structure of Formula (I) or Formula (II) as defined above or a pharmaceutically acceptable salt thereof wherein D- is a drug selected from the group consisting of:—metronidazole, 2-, 3- or 4- clindamycin, lincamycin, ampicillin, amoxicillin, clinafloxacin, delafloxacin, gemifloxacin, nadifloxacin, pazufloxacin, sitafloxacin, sparfloxacin, trovafloxacin, tosufloxacin, ceftabiprole (active metabolite of ceftabiprole medocaril), ceftaroline (active metabolite of ceftaroline fosamil) cefixime, ceftriaxone, cefoperazone, cefotaxime, cefadroxil, donepezil, memantine, miglustat, eliglustat, venlafaxine, desvenlafaxine, fluvoxamine, milnacipran, alendronate, etidronate, pamidronate, neridronate, olpadronate, ibandronate, zoledronate, pazopanib, thioguanine, melphelan, hydroxyurea, temozolomide, metformin, amprenavir, saquinavir, ritonavir, indinavir, nelfinavir, lopinavir, atazanavir, tipranavir, darunavir, didanosine, propafenone and piroxicam, meloxicam, tenoxicam, lornoxicam, acetazolamide, ceftazidime, ciprofloxacin and levofloxacin.

The terminal portion of the amino acid residue or peptide (—R) may be in the form of the free acid i.e. terminating in a —COOH group or may be in a masked (protected) form such as in the form of a carboxylate ester or carboxamide. Sometimes, the amino acid or peptide residue terminates with an amino group.

In an embodiment, the amino acid residue or peptide (—R) terminates with a carboxylic acid group —COOH or an amino group —NH₂. In another embodiment, the residue terminates with a carboxamide group CONR^{a}R^{b}. In an alternate embodiment, the residue terminates with a carboxylate ester COOR^{a}.

In another aspect of the present invention, there is provided a method of treating a disorder in a subject in need thereof. The method comprises orally administering a therapeutically effective amount of a prodrug of the present invention, a proteinogenic and/or non-proteinogenic amino acid and short-chain peptide conjugate of the drug, to the subject. The disorder may be one of a diverse collection of diseases treatable with a relevant drug or drugs amenable to this pro-drugging strategy. Some examples include metronidazole or clindamycin for bacterial infections, propafenone for the treatment of arrhythmias, miglustat or eliglustat for Gauchers disease, donepezil for Alzheimer's disease, piroxicam for rheumatoid arthritis, venlefaxine or fluvoxamine for depression, pazopanib, and melphalan for cancer, alendronate for osteoporosis, amprenavir for viral infections and metformin for diabetes.

In yet another aspect, the present invention is directed to a method for minimizing the gastrointestinal side effects normally associated with administration of a parent drug possessing a derivatisable group (e.g., a hydroxyl, amine or an enolisable carbonyl group). The method comprises orally administering a prodrug or a pharmaceutically acceptable salt thereof to a subject in need thereof wherein upon oral administration the prodrug or pharmaceutically acceptable salt minimizes, if not completely avoids, the gastrointestinal side effects usually seen after oral administration of the unbound drug. The prodrug may have the structure of Formula I, or be a pharmaceutically acceptable salt thereof. The amount of the drug is preferably a therapeutically effective amount.

In yet another aspect, the present invention is directed to a method for extending the sustainment of plasma drug concentrations of a drug having a derivatisable group (e.g., a hydroxyl, amine or an enolisable carbonyl group). The presence of quantities of unhydrolyzed prodrug in plasma provides a reservoir for continued generation of the active drug. Maintenance of plasma drug levels reduces the frequency of drug dosage, and so improves patient compliance. The method comprises administering to a subject in need thereof a prodrug or a pharmaceutically acceptable salt thereof, wherein upon oral administration the extended duration of sustainment is at least 20% greater than that of the drug when administered alone. The prodrug may have the structure of Formula I or be a pharmaceutically acceptable salt thereof. The amount of the prodrug is preferably a therapeutically effective amount.

In yet another aspect, the present invention is directed to a method for increasing the oral bioavailability of a parent drug having a derivatisable group (e.g., a hydroxyl, amine, or an enolisable carbonyl group). The method comprises administering to a subject in need thereof, a prodrug or a pharmaceutically acceptable salt thereof, wherein upon oral administration the bioavailablity is increased at least 20% above that of the drug when administered alone. The prodrug may have the structure of Formula I or be a pharmaceutically acceptable salt thereof. The amount of the prodrug is preferably a therapeutically effective amount.

In yet another aspect, the present invention is directed to a method for reducing the intersubject variability in attained plasma levels of a parent drug having a derivatisable group (e.g., a hydroxyl, amine or an enolisable carbonyl group). The method comprises administering to a subject in need thereof a prodrug or a pharmaceutically acceptable salt thereof, wherein upon oral administration the variability is reduced by at least 20% of that seen when the drug is administered alone. The prodrug may have the structure of Formula I or be a pharmaceutically acceptable salt thereof. The amount of the prodrug is preferably a therapeutically effective amount.

These and other embodiments are disclosed or are apparent from and encompassed by the Detailed Description.

### BRIEF DESCRIPTION OF THE DRAWINGS

FIG. 1 shows the plasma clindmaycin concentrations after giving either the parent drug or clindamycin succinyl valine ester prodrug.

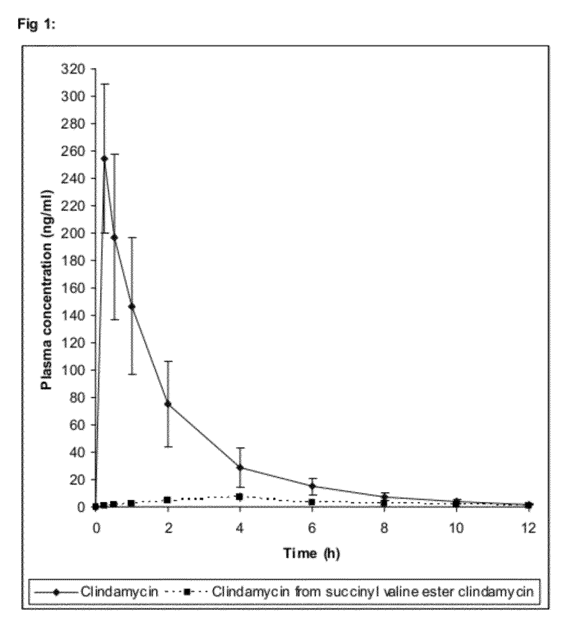

FIG. 2 shows the plasma clindmaycin concentrations after giving either the parent drug or clindamycin glutaryl PABA ester prodrug.

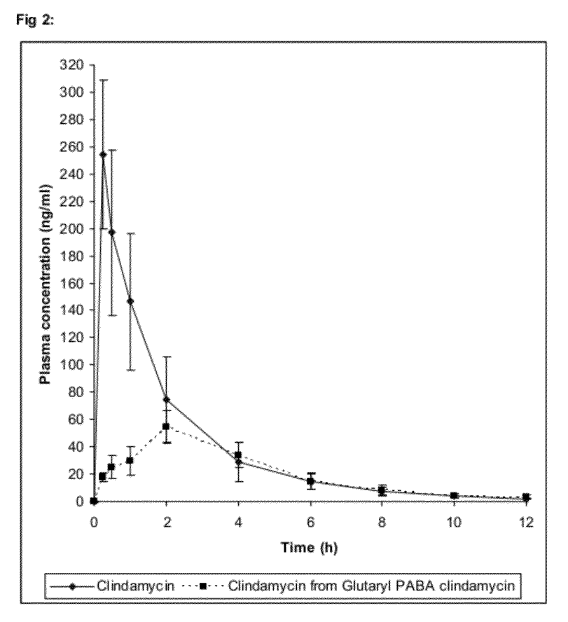

### DETAILED DESCRIPTION OF THE INVENTION

The combinations of the L and R groups contemplated within the scope of the present invention include those in which combinations of variables (and substituents) of the L and R groups are permissible so that such combinations result in stable compounds of Formula (I). For purposes of the present invention, it is understood that the combinations of the variables can be selected by one of ordinary skill in the art to provide compounds of Formula (I) that are chemically stable and that can be readily synthesized by techniques known in the art, as well as those methods set forth in the example section and figures.

The invention encompasses tautomeric forms of the compounds specifically disclosed, as well as geometrical and optical isomers. Thus, when the compounds specifically disclosed include an alkene double bond (for example, compounds having L as

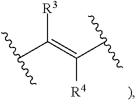

the illustrated structures are intended to include both the E- and Z-geometrical isomers.

The groups R¹, R², R³, R⁴, R⁵ and R⁶ may independently be optionally substituted, where chemically possible, with at each occurrence between 1 and 5 substituents independently selected from the group comprising, F, Cl, Br, NH₂, Me, Et, NO₂, OH and COOH.

The following embodiments apply equally to the first, second and third aspects of the present invention.

In an embodiment, M is

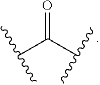

In an alternative embodiment, M is

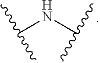

In an embodiment, X is a bond.

In an alternative embodiment, X is —O—.

In an alternative embodiment, X is —NH—.

In an alternative embodiment, X is aryl, preferably phenyl.

In an embodiment, R¹ and R² are each independently selected from the group consisting of: hydrogen, hydroxy, —(CR⁵R⁶)_{q}C(═O)R⁷, —C(═O)R⁷, C₁₋₆ alkyl, aryl, —NR⁵R⁶ and —NR⁵(CO)R⁷; or together with the atom to which they are bonded, R¹ and R² may form a carbonyl or an ethylene, where q, R⁵, R⁶ and R⁷ are as defined above in relation to Formula I.

In preferred embodiment, R¹ and R² are each independently selected from the group consisting of: hydrogen, hydroxy, —(CR⁵R⁶)_{q}C(═O)R⁷, —C(═O)R⁷, C₁₋₆ alkyl and —NR⁵R⁶, preferably q is 1, where R⁵, R⁶ and R⁷ are as defined above in relation to Formula I.

In more preferred embodiment, R¹ and R² are each independently selected from the group consisting of: hydrogen, hydroxy, —(CH₂)C(═O)OH, —C(═O)OH, methyl and —NH₂.

In more preferred embodiment, R¹ and R² are each independently selected from the group consisting of: hydrogen and C₁₋₄ alkyl.

In an embodiment, R³ and R⁴ are each independently selected from the group consisting of: hydrogen, C₁₋₆ alkoxy, —C(═O)R⁷ and C₁₋₆ alkyl, where R⁷ is as defined above in relation to Formula I.

In preferred embodiment, R³ and R⁴ are each independently selected from the group consisting of: hydrogen, methoxy, —C(═O)OH and methyl.

In an embodiment, R⁵ is H.

In an embodiment, R⁶ is H.

In an embodiment, R⁷ is selected from the group consisting of: hydroxyl and C₁₋₆ alkyl.

In a preferred embodiment, R⁷ is hydroxyl.

In an embodiment, n is 2 or 3.

In an embodiment, m is 0.

In an embodiment, p is 0.

In an embodiment, n is an integer of from 0 to 16, m is 0, p is 0 and X is a bond.

In a preferred embodiment, n is an integer of from 2 to 3, m is 0, p is 0 and X is a bond.

In an embodiment, n and p are each independently an integer of from 0 to 1, provided that the sum of n and p is an integer of from 0 to 1, p is 1 and X is a bond.

In an embodiment, n, m and p are all 0 and X is aryl, preferably X is phenyl.

In an embodiment, n is an integer of from 1 to 3, m and p are both 0 and X is selected from the group consisting of: —O— and —NH—.

In an embodiment, M is

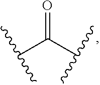

n is an integer of from 0 to 16, m is 0, p is 0 and X is a bond.

In a preferred embodiment, M is

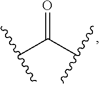

n is an integer of from 2 to 3, m is 0, p is 0 and X is a bond. Preferably, R¹ and R² are each independently selected from the group consisting of: H and C₁₋₄ alkyl, and further preferably R¹ and R² are each independently selected from the group consisting of: H and methyl.

In an embodiment, M is

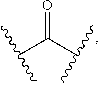

n and p are each independently an integer of from 0 to 1, provided that the sum of n and p is an integer of from 0 to 1, p is 1 and X is a bond.

In an embodiment, M is

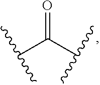

n, m and p are all 0 and X is aryl, preferably X is phenyl.

In an embodiment, M is

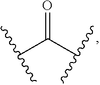

n is an integer of from 1 to 3, m and p are both 0 and X is selected from the group consisting of: —O— and —NH—.

In an embodiment, M is

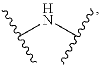

n is an integer of from 2 to 3, m is 0, p is 0 and X is a bond.

In an embodiment, L is

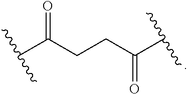

In an embodiment, L is

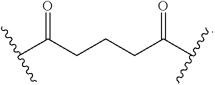

In an embodiment, L is

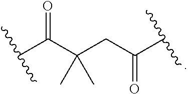

In an embodiment, L is

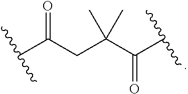

In an embodiment, L is

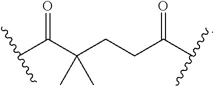

In an embodiment, L is

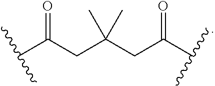

In an embodiment, L is

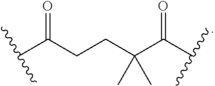

In an embodiment, L is a residue including a dicarboxylic acid moiety selected from those recited in table 1a. It is noted that the actual carboxylic acid (i.e. prior to its attachment between the non-opioid drug and R³) is recited in table 1:

TABLE 1a

| Common Name | IUPAC Name | Chemical Formula |
|---|---|---|
| Oxalic Acid | Ethanedioic Acid | HOOC—COOH |
| Malonic Acid | Propanedioic Acid | HOOC—(CH₂)—COOH |
| Succinic Acid | Butanedioic Acid | HOOC—(CH₂)₂—COOH |
| Glutaric Acid | Pentanedioic Acid | HOOC—(CH₂)₃—COOH |
| Adipic Acid | Hexanedioic Acid | HOOC—(CH₂)₄—COOH |
| Pimelic Acid | Heptanedioic Acid | HOOC—(CH₂)₅—COOH |
| Suberic Acid | Octanedioic Acid | HOOC—(CH₂)₆—COOH |
| Azelaic Acid | Nonanedioic Acid | HOOC—(CH₂)₇—COOH |
| Sebacic Acid | Decanedioic Acid | HOOC—(CH₂)₈—COOH |
| Undecanedioic Acid | Undecanedioic Acid | HOOC—(CH₂)₉—COOH |
| Dodecanedioic Acid | Dodecanedioic Acid | HOOC—(CH₂)₁₀—COOH |
| Brassylic Acid | Tridecanedioic Acid | HOOC—(CH₂)₁₁—COOH |
| | 1,11-Undecanedicarboxylic Acid | |
| Tetradecanedioic Acid | 1,12-Dodecanedicarboxylic Acid | HOOC—(CH₂)₁₂—COOH |
| Pentadecanedioic Acid | 1,15-Pentadecanedioic Acid | HOOC—(CH₂)₁₃—COOH |
| Thapsic Acid | Hexadecanedioic Acid | HOOC—(CH₂)₁₄—COOH |
| | Hexane-1,16-dioic Acid | |
| Heptadecanedioic Acid | 1,15-Pentadecanedicarboxylic Acid | HOOC—(CH₂)₁₅—COOH |
| Octadecanedioic Acid | 1,16-Tetradecanedicarboxylic Acid | HOOC—(CH₂)₁₆—COOH |
| Phthalic Acid | Benzene-1,2-Dicarboxylic Acid | C₆H₄(COOH)₂ |
| Terephthalic Acid | Benzene-1,4-Dicarboxylic Acid | C₆H₄(COOH)₂ |
| Aconitic Acid | Prop-1-ene-1,2,3-tricarboxylic acid | C₆H₆O₆ |
| Achilleic Acid | | |
| Citraconic Acid | 2-methylbut-2-enedioic acid | C₅H₆O₄ |
| Itaconic Acid | Methylenesuccinic Acid | C₅H₆O₄ |
| | 2-Methylidenebutanedioic acid | |
| Aconitic Acid | Prop-1-ene-1,2,3-tricarboxylic acid | C₆H₆O₆ |
| α-Ketoglutaric Acid | 2-oxopentanedioic acid | C₅H₆O₅ |
| N^{α}-Acetyl glutamatic acid | 2-acetamidopentanedioic acid | C₇H₁₁NO₅ |
| Isocitric acid | 1-Hydroxypropane-1,2,3-tricarboxylic acid | C₆H₈O₇ |
| 2-hydroxy-3-methylsuccinic | 2-hydroxy-3-methylsuccinic acid | C₅H₉O₅ |
| acid | | |
| 2-hydroxy-2,3-dimethylsuccinic | 2-hydroxy-2,3-dimethylsuccinic acid | C₆H₁₀O₅ |
| acid | | |
| citric acid | 2-hydroxypropane-1,2,3-tricarboxylic acid | C₆H₈O₇ |

In an embodiment, L is a residue selected from those recited in table 1b.

TABLE 1b

| Name of L residue | Structure |
|---|---|
| N^{a}-Acetyl Aspartic Acid Linker | 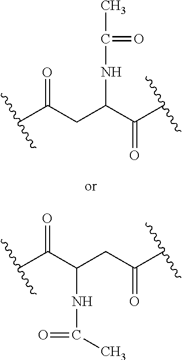 |
| N^{a}-Acetyl Glutamic Acid Linker | 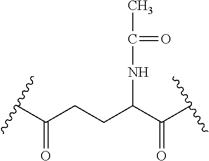 |
| Malic Acid Linker | 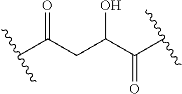 |
| Tartaric Acid Linker | 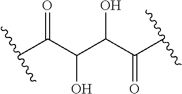 |
| Citramalic Acid Linker | 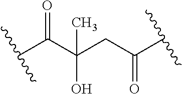 |
| 2-Methyl Succinic Acid Linker | 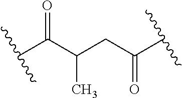 |
| 2,2-Dimethyl Succinic Acid Linker | 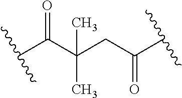 |
| 2,3-Dimethyl Succinic Acid Linker | 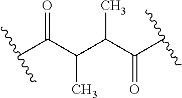 |
| (S)-Citramalic Acid Linker | 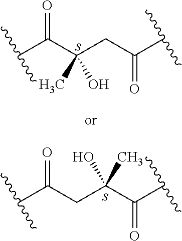 |
| 2-Phenylsuccinic Acid Linker | 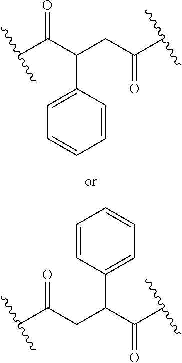 |
| 2,2-Dimethylglutaric Acid Linker | 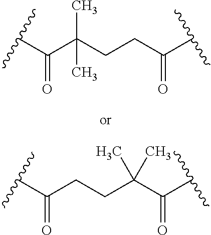 |
| 3,3-Dimethylglutaric Acid Linker | 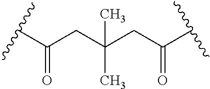 |
| β-Alanine Linker | 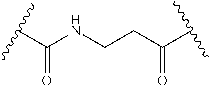 |
| γ-Aminobutyric Acid (GABA) Linker | 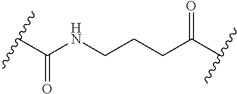 |
| 3-(Carboxy) Butanoic Acid Linker | 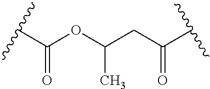 |
| 3-(Carboxy) Propanoic Acid Linker | 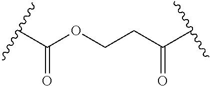 |
| 4-(Carboxy) Butanoic Acid Linker | 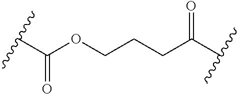 |
| 2-(Carboxy) Propanoic Acid Linker | 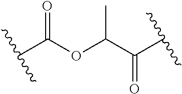 |
| 2-(Carboxy) Acetic Acid Linker | 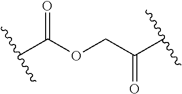 |
| Glutaconic Acid Linker | 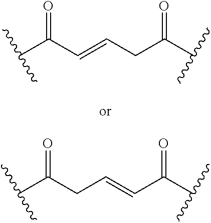 |
| Ketoglutaric Acid Linker | 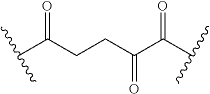 |
| Maleic Acid Linker | 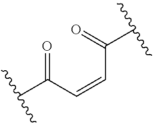 |
| Citraconic Acid Linker | 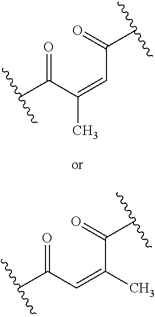 |
| 2,3-Dimethylmaleic Acid Linker | 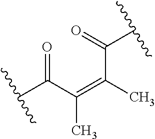 |
| Fumaric Acid Linker | 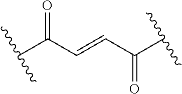 |
| 2,3-Dimethylfumaric Acid Linker | 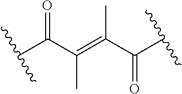 |
| Z-Methoxybutenedioic Acid Linker | 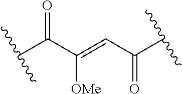 |
| Aconitic Acid Linker | 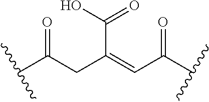 |
| E-Methoxybutenedioc Acid Linker | 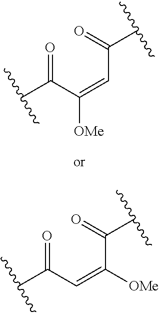 |
| 2-Methylene Glutaric Acid Linker | 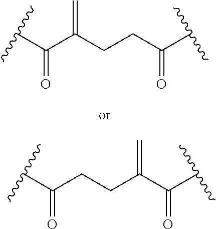 |
| Itaconic Acid Linker | 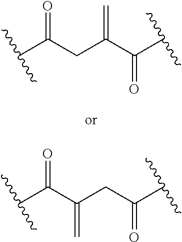 |
| Terephthalic Acid Linker | 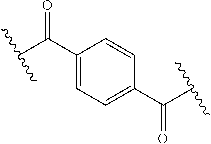 |
| Phthalic Acid Linker | 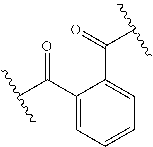 |
| Citroyl Acid Linker | 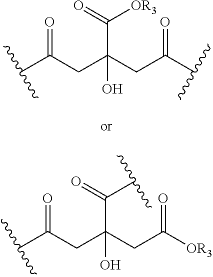 |
| Citric Acid Linker (1) | 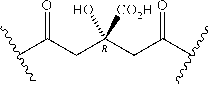 |
| Citric Acid Linker (2) | 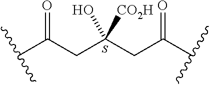 |
| Citric Acid Linker (3) | 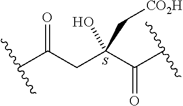 |
| Citric Acid Linker (4) | 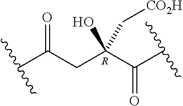 |
| Citric Acid Linker (5) | 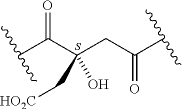 |
| Citric Acid Linker (6) | 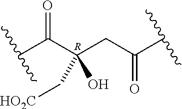 |

In an embodiment of the first and third aspects, —R is an amino acid residue containing from 2 to 20 carbon atoms or a peptide formed from 2 to 10 independently selected amino acids each containing from 2 to 20 carbon atoms. In an embodiment of the first and third aspects, R is an amino acid residue containing from 2 to 20 carbon atoms.

In an embodiment of the second and third aspects, R is —OR^{a}.

In an alternate embodiment of the second and third aspects, R is —NR^{a}R^{b}.

In an embodiment of any of the aspects, R^{a} is selected from the group consisting of: H, Me, Et and cyclopropyl. Preferably, R^{a} is H.

In an embodiment of any of the aspects, R^{b} is selected from the group consisting of: H, Me, Et and cyclopropyl. Preferably, R^{b} is H.

In an embodiment of any of the aspects, r is 0. In an embodiment, r is 1.

In an embodiment of the first and third aspects, the amino acid residue is (S) valine.

In an embodiment of the first and third aspects, the amino acid residue is para amino benzoic acid (PABA).

In an embodiment of the first and third aspects, -L-R has a structure selected from the group consisting of:

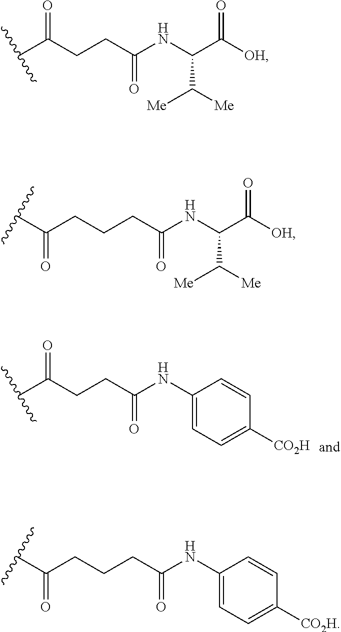

In an embodiment, the amino acid residue is

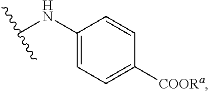

wherein R^{a} is other than H.

In an embodiment, the amino acid residue is

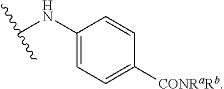

In an embodiment of the first and third aspects, -L-R has a structure selected from the group consisting of:

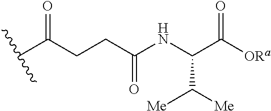

wherein R^{a} is other than H;

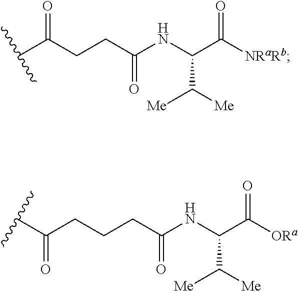

wherein R^{a} is other than H;

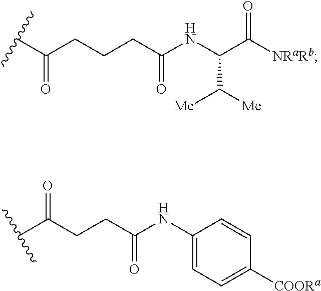

wherein R^{a} is other than H;

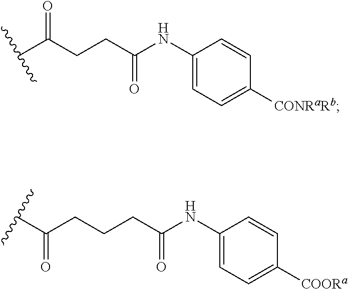

wherein R^{a} is other than H;
and

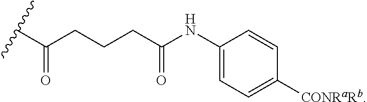

In an embodiment of the second and third aspects, -L-R has the structure selected from the group consisting of:

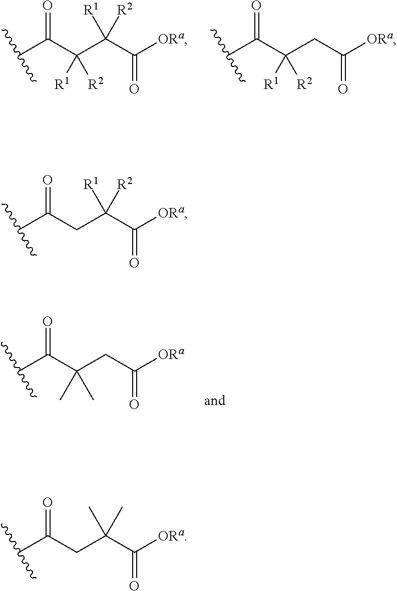

In an embodiment of the second and third aspects, -L-R has the structure selected from the group consisting of:

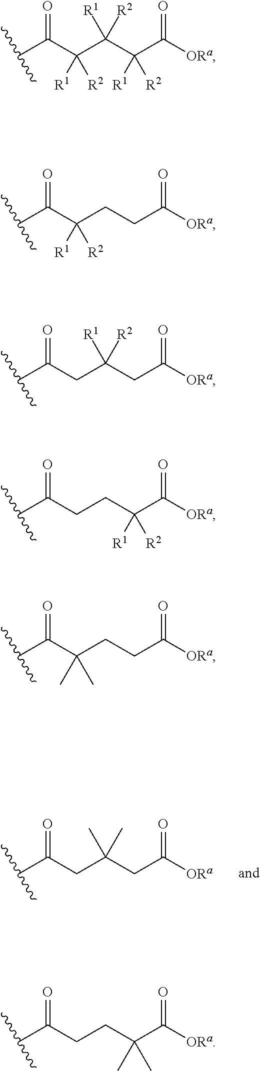

In an embodiment of the second and third aspects, -L-R has the structure selected from the group consisting of:

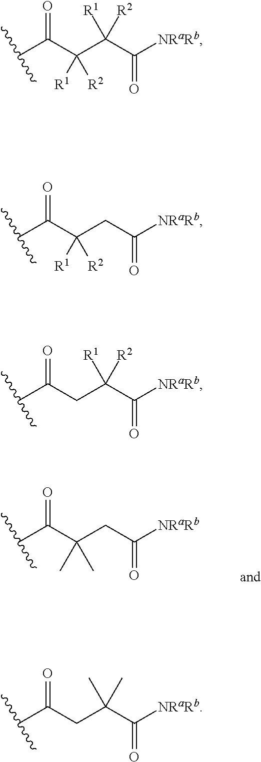

In an embodiment of the second and third aspects, -L-R has the structure selected from the group consisting of:

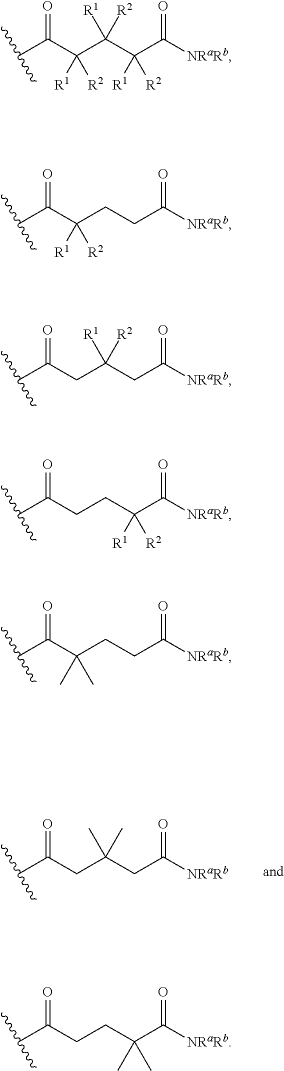

When administered to a subject, the prodrugs of the first and third aspects (i.e. prodrugs having the parent drug compound conjugated to a linker group which is in turn conjugated to a terminal amino acid drug) may metabolise to the prodrugs of the second and third aspects (i.e. prodrugs having the parent drug compound conjugated only to the linking group and no longer having the terminal amino acid or peptide present). The prodrugs of the first and third aspects may therefore generate the prodrugs of the second and third aspects after administration to a subject.

In an embodiment, D is selected from the group consisting of: metronidazole, 2-, 3- or 4-clindamycin, lincomycin, ampicillin, amoxicillin, clinafloxacin, delafloxacin, gemifloxacin, nadifloxacin, pazufloxacin, sitafloxacin, sparfloxacin, trovafloxacin, tosufloxacin, ceftobiprole (active metabolite of ceftobiprole medocaril), ceftaroline (active metabolite of ceftaroline fosamil) cefixime, ceftriaxone, cefoperazone, cefotaxime, cefadroxil, donepezil, memantine, miglustat, eliglustat, venlafaxine, desvenlafaxine, fluvoxamine, milnacipran, alendronate, etidronate, pamidronate, neridronate, olpadronate, ibandronate, zoledronate, pazopanib, thioguanine, melphelan, hydroxyurea, temozolomide, metformin, amprenavir, saquinavir, ritonavir, indinavir, nelfinavir, lopinavir, atazanavir, tipranavir, darunavir, didanosine, propafenone, piroxicam, meloxicam, tenoxicam, lornoxicam, acetazolamide, ceftazidime, ciprofloxacin and levofloxacin.

In an embodiment, D is selected from the group consisting of: metronidazole, 2-, 3- or 4-clindamycin, lincomycin, ampicillin, amoxicillin, clinafloxacin, delafloxacin, gemifloxacin, nadifloxacin, pazufloxacin, sitafloxacin, sparfloxacin, trovafloxacin, tosufloxacin, ceftobiprole (active metabolite of ceftobiprole medocaril), ceftaroline (active metabolite of ceftaroline fosamil) cefixime, ceftriaxone, cefoperazone, cefotaxime, cefadroxil, donepezil, memantine, miglustat, eliglustat, venlafaxine, desvenlafaxine, fluvoxamine, milnacipran, alendronate, etidronate, pamidronate, neridronate, olpadronate, ibandronate, zoledronate, pazopanib, thioguanine, melphelan, hydroxyurea, temozolomide, metformin, amprenavir, saquinavir, ritonavir, indinavir, nelfinavir, lopinavir, atazanavir, tipranavir, darunavir, didanosine, propafenone, piroxicam, meloxicam, tenoxicam, lornoxicam and acetazolamide.

In an embodiment, D is selected from the group consisting of: metronidazole, 2-, 3- or 4-clindamycin, lincomycin, ampicillin, amoxicillin, clinafloxacin, delafloxacin, gemifloxacin, nadifloxacin, pazufloxacin, sitafloxacin, sparfloxacin, trovafloxacin, tosufloxacin, ceftobiprole (active metabolite of ceftobiprole medocaril), ceftaroline (active metabolite of ceftaroline fosamil) cefixime, ceftriaxone, cefoperazone, cefotaxime, cefadroxil, donepezil and memantine.

In an embodiment, the drug is derivatised by attaching the linker to a hydroxyl group and is selected from the group consisting of: metronidazole, 2-, 3- or 4-clindamycin, lincomycin, delafloxacin, nadifloxacin, ceftobiprole (active metabolite of ceftobiprole medocaril), cefoperazone, cefadroxil, miglustat, eliglustat, venlafaxine, desvenlafaxine, alendronate, etidronate, pamidronate, neridronate, olpadronate, ibandronate, zoledronate, hydroxyurea, amprenavir, saquinavir, ritonavir, indinavir, nelfinavir, lopinavir, atazanavir, tipranavir, darunavir, didanosine, propafenone, piroxicam, meloxicam, tenoxicam and lornoxicam.

In an embodiment, the drug is derivatised by attaching the linker to a hydroxyl group and is selected from the group consisting of: metronidazole, 2-, 3- or 4-clindamycin, lincomycin, delafloxacin, nadifloxacin, ceftobiprole (active metabolite of ceftobiprole medocaril), cefoperazone and cefadroxil.

In an embodiment, the drug is derivatised by attaching the linker to an amine (1° or 2°) and is selected from the group consisting of: ampicillin, amoxicillin, clinafloxacin, delafloxacin, gemifloxacin, pazufloxacin, sitafloxacin, sparfloxacin, trovafloxacin, tosufloxacin, ceftobiprole (active metabolite of ceftobiprole medocaril), ceftaroline (active metabolite of ceftaroline fosamil) cefixime, ceftriaxone, cefotaxime, cefadroxil, memantine, fluvoxamine, milnacipran, alendronate, pamidronate, neridronate, pazopanib, thioguanine, melphelan, hydroxyurea, temozolomide, metformin, amprenavir, saquinavir, darunavir, and acetazolamide.

In an embodiment, the drug is derivatised by attaching the linker to an amine (1° or 2°) and is selected from the group consisting of: ampicillin, amoxicillin, clinafloxacin, delafloxacin, gemifloxacin, pazufloxacin, sitafloxacin, sparfloxacin, trovafloxacin, tosufloxacin, ceftobiprole (active metabolite of ceftobiprole medocaril), ceftaroline (active metabolite of ceftaroline fosamil) cefixime, ceftriaxone, cefotaxime, cefadroxi and memantine.

In an embodiment, the drug is derivatised by attaching the linker to the oxygen of an enolisable carbonyl and is selected from the group consisting of: donepezil, tipranavir and propafenone. Most preferably the drug is donepezil.

In some instances there may be more than one possible position of derivatisation in a drug molecule to which the prodrugging entity (e.g. a dicarboxylate bridge+amino acid(s)) may be attached. For example the drug may contain: more than one hydroxyl group; more than one amino group; more than one enolisable carbonyl group; a hydroxyl and an amino group; a hydroxyl and an enolisable carbonyl group; an amino group and an enolisable carbonyl group; or a hydroxyl, amino and an enolisable group in the same molecule. In an embodiment, when the drug molecule includes more than one possible position of derivatisation, the prodrug of the invention has only a single -L-R group conjugated to the drug molecule. The present invention embraces any position of substitution by -L-R on the drug molecule. Preferably, the position nearest the pharmacophoric region (when that is known) is conjugated to -L-R.

In an embodiment, D is selected from the group consisting of:

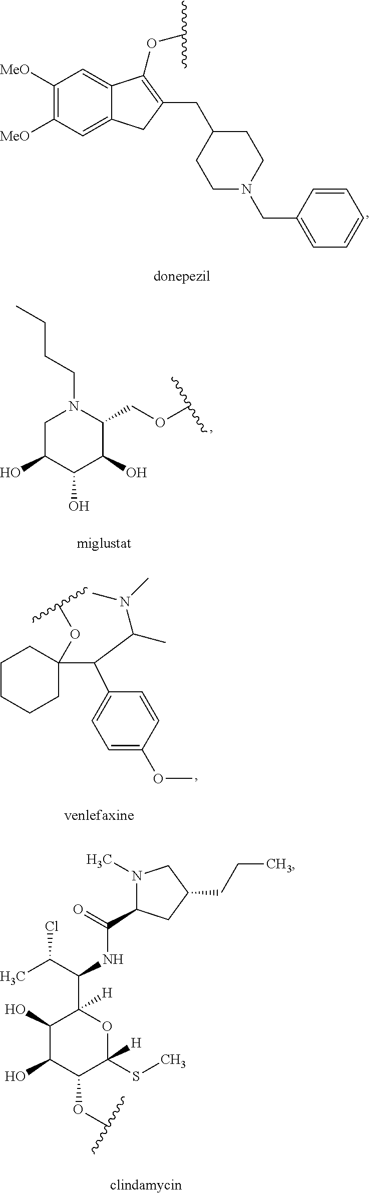

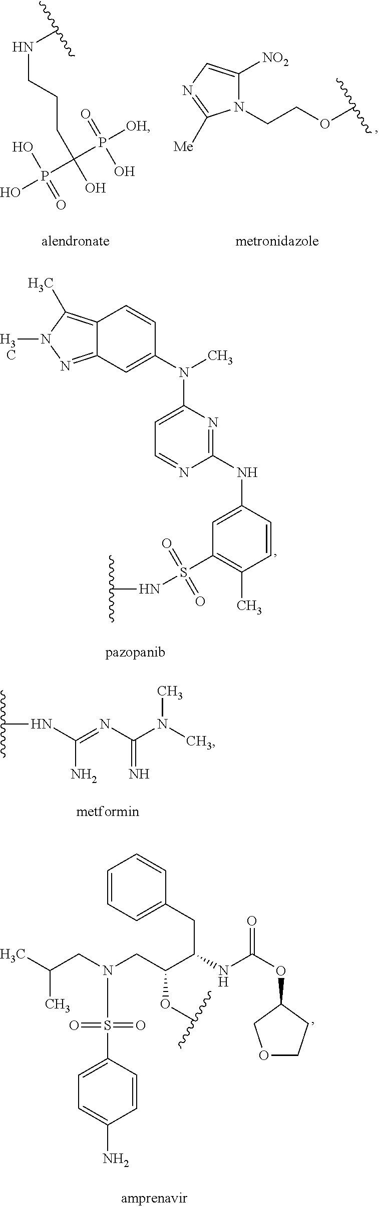

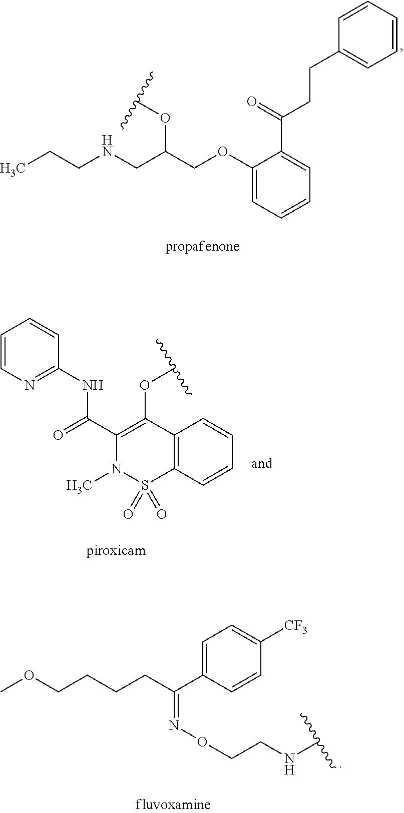

In an embodiment, the prodrug is a prodrug of donepezil and has the formula:

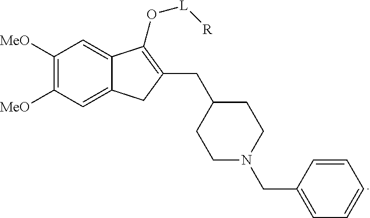

Preferably the prodrug has the formula:

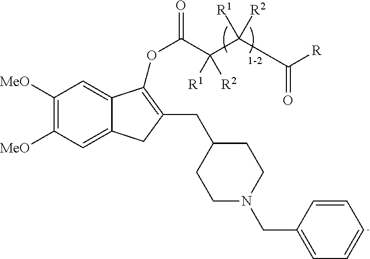

Preferably, R¹ and R² are selected from the group consisting of: H and C₁₋₄alkyl. Preferably, R is selected from the group consisting of: —OR^{a} and —NR^{a}R^{b}.

In an embodiment, the systemic disease treatable using a prodrug of Formula I includes a disorder selected from the group consisting of: a bacterial infection, an arrhythmia, Gauchers disease, Alzheimer's disease, rheumatoid arthritis, depression, cancer, osteoporosis, a viral infection and diabetes.

In one embodiment, the drug, D, is anyone one of a diverse collection for treating a variety of different diseases such as Alzheimer's drugs, e.g. donepezil, antibiotics, e.g. metronidazole, non-steroidal anti-inflammatory drugs, e.g. piroxicam, and Gauchers disease, e.g. miglustat.

In an embodiment, the prodrug is a single amino acid prodrug of donepezil. Single amino acid prodrugs of donepezil include donepezil-[succinyl-(S)-isoleucine]enol ester, donepezil-[succinyl-(S)-leucine]enol ester, donepezil-[succinyl-(S)-aspartic acid]enol ester, donepezil-[succinyl-(S)-methionine]enol ester, donepezil-[succinyl-(S)-histidine]enol ester, donepezil-[succinyl-(S)-tyrosine]enol ester, donepezil-[succinyl-(S)-phenylalanine]enol ester, donepezil-[succinyl-(S)-serine]enol ester, donepezil-[glutaryl-(S)-valine]enol ester, and donepezil-[glutaryl-(S)-leucine]enol ester. Similar conjugates in which the donepezil above is replaced by one of the aforementioned drugs can also be prepared in accordance with the invention. In the donepezil-[succinyl-(S)-serine]enol ester and donepezil-[succinyl-(S)-tyrosine]enol ester prodrugs, the amino acid portions are conjugated to the succinyl linker via the amino portion rather than the phenolic/hydroxy portions.

In an embodiment, the prodrug is a donepezil dipeptide prodrug. Dipeptide donepezil prodrugs include donepezil-[succinyl-(S)-valine-valine]enol ester, donepezil-[succinyl-(S)-isoleucine-isoleucine]enol ester and donepezil-[succinyl-(S)-leucine-leucine]enol ester. Similar conjugates in which the donepezil above is replaced by one of the aforementioned drugs can also be prepared in accordance with the invention.

In an embodiment, the prodrug is dicarboxylic acid linked prodrug of fluvoxamine. Preferably, the prodrug is fluvoxamine-glutaryl-PABA or fluvoxamine-succinyl-valine:

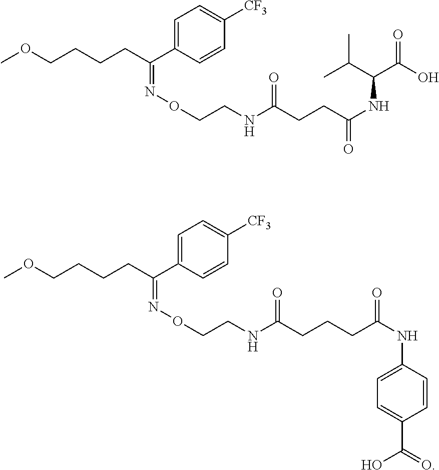

Definitions:

The term “amino acid residue” is intended to mean a moiety having an amine group and a carboxylic acid group. The amino acid residue may have one or more amine groups and one or more carboxylic acid groups. Thus the term “amino acid residue” is intended to include both natural and synthetic amino acids. The class of natural amino acids includes both proteinogenic amino acids and also naturally occurring non-proteinogenic amino acids. These naturally occurring non-proteinogenic amino acids are those that may be found, for example, in the body or in food stuffs, but which do not participate in protein biosynthesis. There are twenty-two proteinogenic amino acids and of the twenty-two, only twenty are directly encoded by the universal genetic code. The remaining two, selenocysteine and pyrrolysine, are incorporated into proteins by unique synthetic mechanisms. The invention is intended to encompass the twenty universally encoded amino acids plus the remaining two mentioned above. The term “amino acid residue” is therefore intended to include the following: Alanine, Cysteine, Aspartic Acid, Glutamic Acid, Phenylalanine, Glycine, Histidine, Isoleucine, Lysine, Leucine, Methionine, Asparagine, Proline, Glutamine, Arginine, Serine, Threonine, Valine, Tryptophan, Tyrosine, Selenocysteine and Pyrrolysine.

The amino acid residue can be bound to L via a suitable carboxylic acid, amine, hydroxyl, thiol, guanidine or carboxamide group on its side chain or alternatively the carboxylic acid or amine group of the non-side chain portion of the amino acid. Thus, if the amino acid residue were, for example, arginine, cysteine, glutamine or tyrosine the amino acid can be bound to L at any one of the positions illustrated in the figure below:

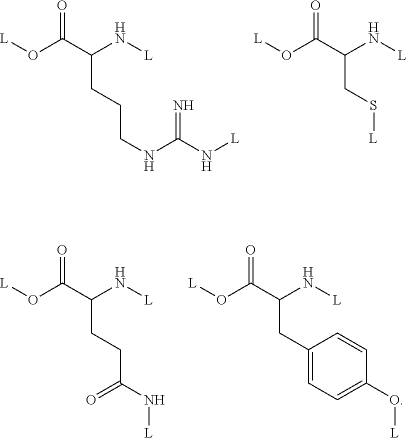

In addition to amino acid residues having a terminal carboxylic acid or amine group, the term is also intended to include: an amino acid alkyl ester (e.g. an amino acid C₁₋₆ alkyl ester); an amino acid aryl ester; an N-alkylated amino acid (e.g. a C₁₋₆ N-alkylated amino acid such as N-methylated amino acid or an N-methylcyclopropylated amino acid); an N,N-dialkylated amino acid (e.g. a C₁₋₆ N,N-dialkylated amino acid, which can include N,N-dimethylcyclopropylated amino acids), preferably the N,N-dialkylated amino acid is an N,N-dimethylated amino acid; an N-acylated amino acid (e.g. a C₁₋₆ N-acylated amino acid); an N-arylated amino acid; an N-alkylated amino acid ester; an N-acylated amino acid ester; an N-arylated amino acid ester; an O-alkylated amino acid (e.g. a C₁₋₆₀-alkylated amino acid); an O-arylated amino acid; an O-acylated amino acid; an O-alkylated amino acid ester; an O-arylated amino acid ester; an O-acylated amino acid ester; an S-alkylated amino acid; an S-acylated amino acid; an S-arylated amino acid; an S-alkylated amino acid ester; an S-acylated amino acid ester; or an S-arylated amino acid ester. In other words, the invention also envisages amino acid derivatives such as those mentioned above which have been functionalized by simple synthetic transformations known in the art (e.g. as described in “Protective Groups in Organic Synthesis” by TW Greene and PGM Wuts, John Wiley & Sons Inc (1999), and references therein. Of course, in N,N-dialkylated amino acids, the alkyl groups may be the same or different.

In addition, the side chains of the above amino acids can be in either the (R) or the (S) configuration. In other words, both L- and D-amino acids are within the scope of the present invention, though the D-amino acids are of course not naturally occurring.

As mentioned above, the term “amino acid residue” also includes non-proteinogenic amino acids such as amino acids which can be incorporated into proteins during translation (including pyrrolysine, ornithine and selenocysteine). The term “non-proteinogenic amino acid” also includes homologues of proteinogenic amino acids such as, but not limited to, homoarginine. The term “non-proteinogenic amino acid” also includes beta amino acids such as, but not limited to, beta alanine. The term “amino acid” also includes lactam analogues of natural amino acids such as, but not limited to, pyroglutamine.

A “non-proteinogenic amino acid” is an organic compound which is an amino acid, but is not among those encoded by the standard genetic code, or incorporated into proteins during translation. Non-proteinogenic amino acids, thus, include amino acids or analogues of amino acids other than the 20 proteinogenic amino acids and include, but are not limited to, the D-isostereomers of proteinogenic amino acids. Examples of non-proteinogenic amino acids include, but are not limited to: citrulline, homocitrulline, hydroxyproline, homoarginine, homoserine, homotyrosine, homoproline, ornithine, 4-amino-phenylalanine, sarcosine, biphenylalanine, homophenylalanine, 4-nitro-phenylalanine, 4-fluoro-phenylalanine, 2,3,4,5,6-pentafluoro-phenylalanine, norleucine, cyclohexylalanine, N-acetic acid, O-methyl serine (i.e., an amino acid side chain having the formula

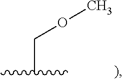

acetylamino alanine (i.e., an amino acid side chain having the formula

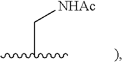

β-alanine, β-(acetylamino)alanine, β-aminoalanine, β-chloroalanine, α-aminoisobutyric acid, N-methyl-alanine, N-methyl-glycine, N-methyl-glutamic acid, tert-butylglycine, α-aminobutyric acid, α-aminoisobutyric acid, acedic acid, 2-aminoisobutyric acid, 2-aminoindane-2-carboxylic acid, selenomethionine, lanthionine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, cyclohexylglycine, diethylglycine, dipropylglycine and derivatives thereof wherein the amine nitrogen has been mono- or di-alkylated. Other examples of non-proteinogenic amino acids include para amino benzoic acid (PABA), 5-amino salicylic acid (5-ASA) and 4-amino salicylic acid (4-ASA).

The term “peptide” refers to an amino acid chain consisting of 2 to 10 amino acids (bound via peptide bonds), unless otherwise specified. In preferred embodiments, the peptide used in the present invention is 2 or 3 amino acids in length. In this embodiment, at least one amino acid side chain in the peptide is bound to another amino acid (either through one of the termini or the side chain).

The term “amino” refers to a —NH₂ group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

The term “substituted alkyl” as used herein denotes alkyl radicals wherein at least one hydrogen is replaced by one more substituents such as, but not limited to, hydroxy, carboxyl alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms selected from nitrogen, phosphorus, oxygen and sulphur.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, carboxyl, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., −C(O)O—R where R is an alkyl such as methyl) and acyloxyester (e.g., —OC(O)—R where R is an alkyl such as methyl). The definition pertains whether the term is applied to a substituent itself or to a substituent of a substituent.

The terms “keto” and “oxo” are synonymous, and refer to the group ═O.

The term “carbonyl” refers to a group —C(═O).

The term “enolisable carbonyl” refers to a group having the structure —CHR—C(═O)— or —C(═O)—CHR— in which the alpha-hydrogen can be removed to form an enolate using a base taken from the range of bases readily available to the synthetic chemist. The skilled person will readily understand what is meant by the term enolisable carbonyl and could easily identify enolisable carbonyl groups on a drug molecule.

The term “carboxyl” refers to a group —CO₂H and consists of a carbonyl and a hydroxyl group (more specifically, C(═O)OH).

The term “linker” for the purposes of the present invention, refers to the group between the drug, D, and —OR^{a} or —NR^{a}R^{b}, or the amino acid/peptide moiety.

Regarding the linker, the left hand carbonyl group of the linker (as drawn above) is bound to an oxygen or nitrogen atom in the drug, while the right hand carbonyl (as drawn above) or amine of the linker is bound to —OR^{a} or —NR^{a}R^{b}, or the peptide or amino acid.

Prodrug moieties described herein may be referred to based on their linkage structure and their amino acid or peptide portion (if they have an amino acid or peptide portion). The amino acid or peptide in such a reference should be assumed to be bound via an amino, hydroxyl or carboxylic acid terminus on the amino acid or peptide to one carbonyl (originally part of a carboxyl group of the linker) or one amine group of the linker while the other end of the linker is attached to the drug via an ester or amide bond, unless otherwise specified. The linker may or may not be variously substituted as stipulated earlier.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to the drug portion of a prodrug of the present invention, as described herein.

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. “Pharmaceutical Salts,” J. Pharma. Sci. 1977; 66:1).

Compounds of the Invention

An —OH (hydroxyl) group can be esterified with a dicarboxylic acid such as, but not limited to, malonic, succinic, glutaric, adipic or other longer chain dicarboxylic acid, or substituted derivative thereof (for example, see Tables 1 and 2). In addition, a keto group can be enolized and then esterifed with a dicarboxylic acid such as the ones described above. Furthermore, an amino group (—NH₂) can be reacted with a dicarboxylic acid by to form a peptide bond. The amino acid or peptide may then be attached to the remaining carboxyl group via the N-terminal nitrogen on the peptide/amino acid, or a nitrogen present in an amino acid side chain (e.g., a lysine side chain) although other connectivities are possible.

Another embodiment is directed to prodrugs linked to an amino acid or peptide through a dicarboxylic acid linker having a double bond. In this embodiment, maleic acid, fumaric acid, citraconic acid, aconitic acid, crotonic acid or glutaconic acid can be used as a dicarboxylic acid linker. In a further embodiment, the proteinogenic amino acid side chain is selected from valine, leucine and isoleucine.

Yet another embodiment is directed to prodrugs linked to an amino acid or peptide through a substituted maleic acid, fumaric acid, or citraconic acid dicarboxylic acid linker. In a further embodiment, the linker is selected from 3,3-dimethylmaleic acid, 2,3-dimethylfumaric acid, Z-methoxybutenedioic acid and E-methoxybutenedioic acid.

Itaconic acid, ketoglutaric and 2-methylene glutaric acid can also be used as a dicarboxylic acid linker in some embodiments. Here, R₁ and R₂ on one of the carbons of the linker, L, taken together, is a methylene group.

In one embodiment, the prodrug of the present invention is linked to an amino acid or peptide through a dicarboxylic acid linker having an aromatic ring. For example phthalic acid (benzene-1,2-dicarboxylic acid) and terephthalic acid (benzene-1,4-dicarboxylic acid) can be used as a dicarboxylic acid linker.

Still, another embodiment includes prodrugs linked to a peptide or amino acid through a dicarboxylic acid linker substituted with an acetyl

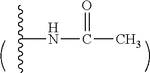

group or a carboxylic acid group. In even a further embodiment, the dicarboxylic acid linker is further substituted with an

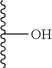

group.

In one embodiment, the drug is linked to a peptide or prodrug through a citric acid linker. The citric acid linker can be any one of 6 isomers, as provided herein in Table 1b.

Peptides comprising any of the proteinogenic amino acids, as well as non-proteinogenic amino acids, can be used in the present invention. Examples of non-proteinogenic amino acids are given above. Non-proteinogenic amino acids can be present in a peptide with only non-proteinogenic amino acids, or alternatively, with both proteinogenic and non-proteinogenic amino acids.

When a ketone is present in the drug scaffold, as stated above, the ketone can be converted to its corresponding enolate and reacted with a modified peptide reactant (which can be a modified amino acid) to form a prodrug. Upon peptide cleavage, the prodrug will revert back to the original drug molecule, with the keto group present.

In a preferred embodiment, the dicarboxylic acid linker is succinic acid. Other dicarboxylic acid linkers within the scope of the invention include, but are not limited to malonic acid, glutaric acid, adipic acid, or other longer chain dicarboxylic acids or substituted derivatives thereof (see Tables 1 and 2).

As alternatives to the use of an unsubstituted dicarboxylic acid linker to attach the drug to the amino acid or peptide prodrug moiety, substituted dicarboxylic acid linkers may be employed. For example, methyl malonic acid may be used. Such substituted dicarboxylic acid linkers would preferably be naturally occurring in the subject to be treated, i.e., non-xenobiotic. Suitable substituted dicarboxylic acids are given in Table 1b.

Antibiotics Prodrugs of the Present Invention e.g. Metronidazole

Metronidazole-[succinyl-(S)-valine]ester

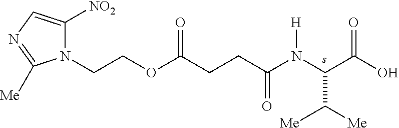

Metronidazole-(glutaryl-PABA) ester

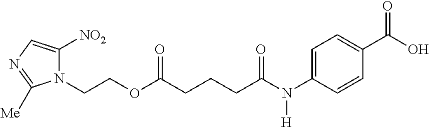

Additionally similar conjugates can be made of the following antibiotics:—, fluoroquinolones including clinafloxacin, delafloxacin, gemifloxacin, nadifloxacin, pazufloxacin, sitafloxacin, sparfloxacin, trovafloxacin, and tosufloxacin; other antibiotics include: 2-, 3- or 4-clindamycin, lincamycin, ampicillin, amoxicillin, and cephalosporins such as cefixime, ceftriaxone, cefoperazone, cefotaxime, cefadroxil, ceftabiprole, (active metabolite of ceftabiprole medocaril), ceftaroline (active metabolite of ceftaroline fosamil). In some cases, instead of an ester linkage to an hydroxy group in the drug molecule, the dicarboxylate bridge is attached via an amide linkage to any free amino function in the drug.

Class 1 Anti-Arrhythmic Prodrugs (Excluding Mexiletine) of the Present Invention e.g. Propafenone Prodrugs

Propafenone[succinyl-(S)-valine]ester

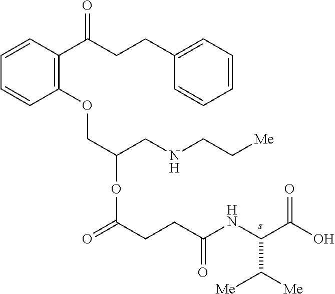

Propafenone (glutaryl-PABA) ester

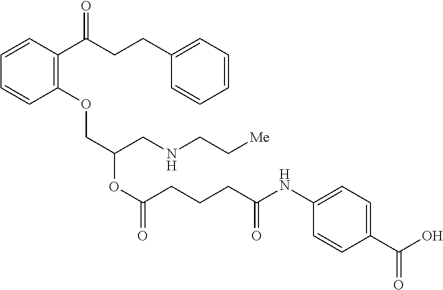

Alternatively propafenone may be conjugated via enolisation of the carbonyl group as shown below:

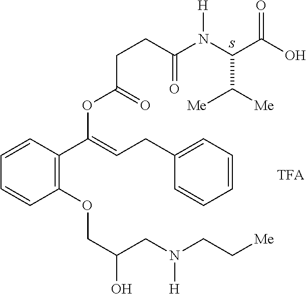

Propafenone[succinyl-(S)-valine]enol ester Trifluoroacetate

Glucosylceramide Synthase Inhibitor Prodrugs of the Present Invention e.g. Miglustat Prodrugs

Miglustat 2-succinyl valine

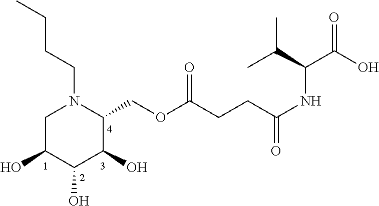

Miglustat 2-glutaryl PABA

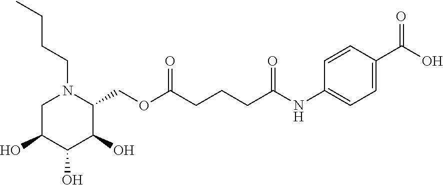

Other positions of substitution around the ring are, of course, possible.

Additionally similar conjugates can be made with comparable GI advantages for another glucosylceramide synthase inhibitor, eliglustat.

Acetyl Choline Esterase Inhibitor (Excluding Galantamine) Prodrugs of the Present Invention—e.g. Donepezil

Additionally similar conjugates can be made of other Alzheimer's drugs including memantine. In this case the dicarboxylate bridge is attached via an amide linkage to the free primary amino function in the drug molecule.

In a preferred embodiment, the single amino acid prodrug of donepezil is the trifluoroacetate salt shown below).

Donepezil-[succinyl-(S)-valine]enol ester

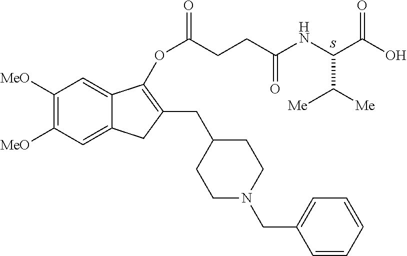

Donepezil (glutaryl-PABA) enol ester

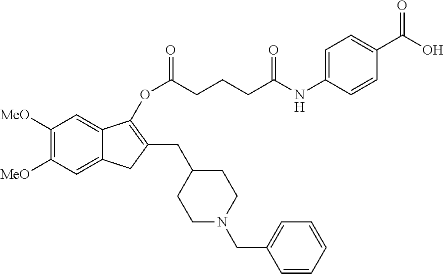

Donepezil-(Glutaryl) enol ester

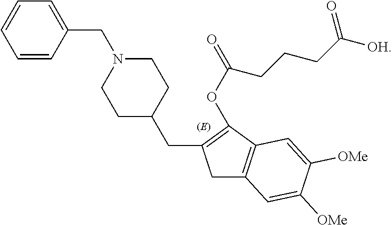

Donepezil-(Succinyl) enol ester

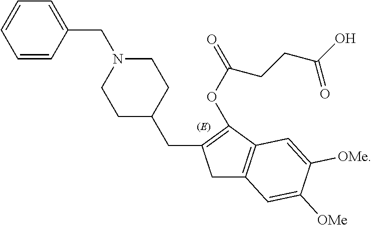

Donepezil-(Succinamido) enol ester

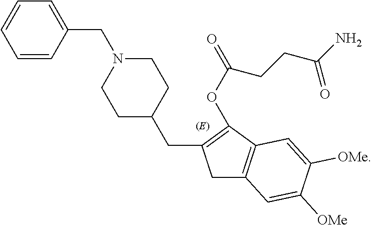

Donepezil-(3,3-dimethylsuccinyl) enol ester

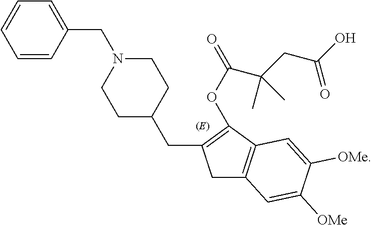

Donepezil-(2,2-dimethylsuccinyl) enol ester

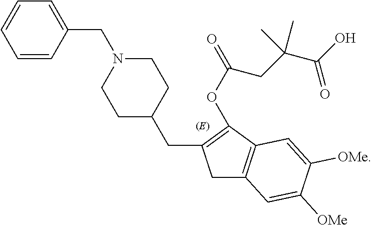

Donepezil-(Glutarylamido) enol ester

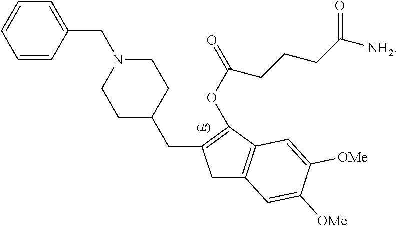

Donepezil-(3,3-dimethylglutaryl) enol ester

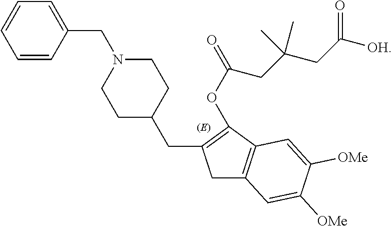

Non Steroidal Anti-Inflammatory Prodrugs of the Present Invention e.g. Piroxicam Prodrugs

Piroxicam 2-succinyl valine

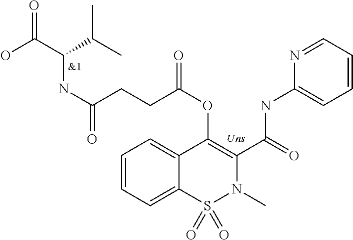

Piroxicam 2-glutaryl PABA

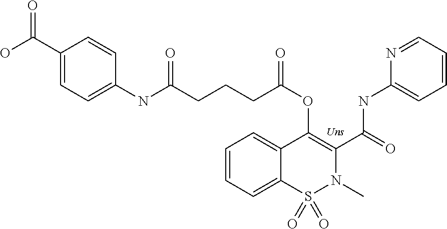

Additionally similar conjugates with comparable GI advantages can be made of the following NSAID's; meloxicam, tenoxicam and lornoxicam.

Selective Serotonin (±Noradrenalin Re-Uptake) Inhibitor Prodrugs of the Present Invention e.g. Venlafaxine Prodrugs

Venlafaxine succinyl valine ester

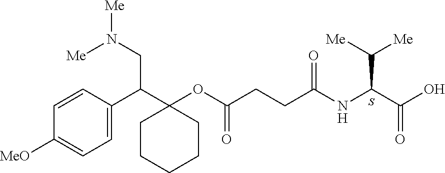

Venlafaxine (glutaryl-PABA) ester

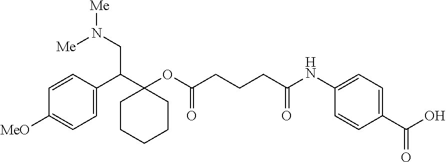

Additionally similar conjugates with comparable GI advantages can be made of the following SSRI/SNRI's; desvenlafaxine, fluvoxamine and milnacipran.

Anticancer Agents (e.g. Tyrosine Kinase Inhibitors) Prodrugs of the Present Invention e.g. Pazopanib Prodrug)

Pazopanib[succinyl-(S)-valine]amide

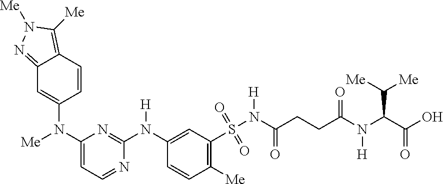

Pazopanib (glutaryl-PABA) amide

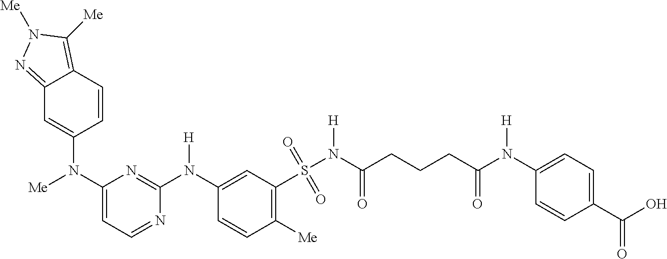

Additionally similar conjugates with comparable GI advantages can be made of the following anticancer agents; thioguanine, melphelan, hydroxyurea and temozolomide.

Bisphosphonates Prodrugs of the Present Invention e.g. Alendronate Prodrugs

Alendronate[Succinyl-(S)-valine]amide

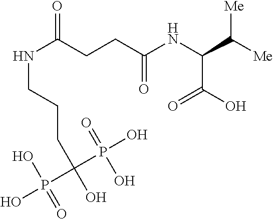

Alendronate (glutaryl-PABA) amide

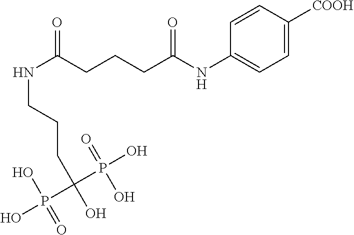

Additionally similar conjugates with comparable GI advantages can be made of the following bisphosphonates: etidronate, pamidronate, neridronate, olpadronate, ibandronate, and zoledronate.

Antiviral Prodrugs of the Present Invention e.g. Amprenavir

Amprenavir[Succinyl-(S)-valine]ester

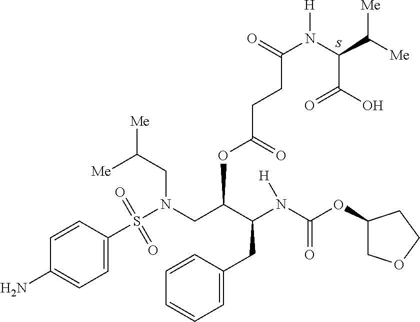

Amprenavir (glutaryl-PABA) ester

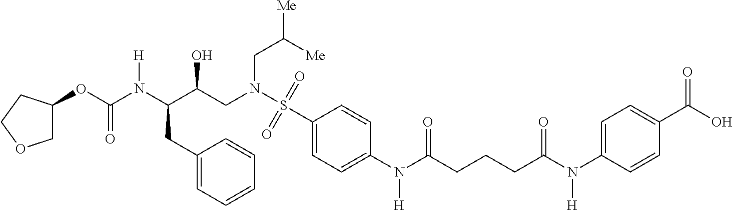

Additionally similar conjugates with comparable GI advantages can be made of the following antiviral agents; didanosine, saquinavir, ritonavir, indinavir, nelfinavir, lopinavir, atazanavir, tipranavir, and darunavir.

Biguanide Antidiabetic Agents Prodrugs of the Present Invention e.g. Metformin Prodrugs

Metformin[succinyl-(S)-valine]amide

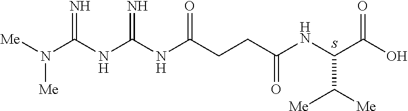

Metformin (glutaryl-PABA) amide

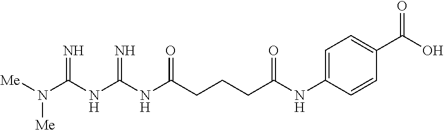

Advantages of the Compounds of the Invention

Without wishing to be bound to any particular theory, it is believed that derivatisation of a suitable drug through a dicarboxylate linker (or suitable derivative as described hereinabove) to an amino acid profoundly changes the spatial structure and physicochemical properties of the derivatised drug rendering it less pharmacologically active than the parent drug. This may also conceivably apply to the attachment of just the dicarboxylate portion (or derivative thereof). Such prodrugs of weakly basic drugspossess both a free terminal carboxyl residue (from the dicarboxylic acid linker moiety) and the basic portion of the parent drug and thus will result in the prodrug molecule becoming zwitterionic. At physiological pH both the drug's basic centre and the prodruging moiety acidic centre may be ionised resulting in a molecule which will less effectively traverse cell membranes and reach those pharmacological receptors within the GI tract. However, absorption of the prodrug from the GI tract will, it is belived, be largely unimpeded since this is likely effected by one or more nutrient transporters, especially for prodrugs having a terminal amino acid group. Another contributory mechanism for such prodrugs being inactive is the likely three dimensional distortion of the drug molecule resulting from the attachment of a medium or longer chain linker to which is appended a terminal amino acid or peptide residue. This is likely to adversely affect the binding or interaction with the pharmacological receptor and hence pharmacological activity. This is especially likely when the conjugate entity is attached at a position in the drug molecule close to the so-called “pharmacophoric region” i.e. that portion which binds directly to the pharmacological receptor. Reduction of the adverse GI side-effects, associated with the oral administration of so many drugs, would be a particular advantage of using a prodrug of the present invention. Inactivation of the drug, although transient and ultimately reversible, serves to minimize any direct effects the drug may normally have while resident in the gut lumen and so avoid any potential for adverse GI events mediated in this way. Oral administration of a temporarily inactivated drug would, during the absorption process of the intact prodrug, preclude access of active drug species to the relevant receptor(s)/enzymes within the gut wall. Alternatively, in the case of antibiotics, the gut microfloral population would be protected from the direct local actions of the drug. In the case of cytotoxics, the integrity of the gut epithelial cells would be preserved if only inactive prodrug was present in the lumen. In the case of NSAID's the direct irritant properties of these drugs may be avoided by the use of such prodrugs. Once absorbed however, the prodrug of the present invention would be metabolized by plasma and liver esterases/peptidases/amidases to the pharmacologically active species, which can then elicit the desired therapeutic effects.

Furthermore, and again without wishing to be bound to any particular theory, it is believed that the amino acid or peptide portion of some of the prodrugs of the present invention (e.g., the amino acid or peptide portion of any of Formula I) may selectively exploit the inherent di- and tripeptide transporter Pept1 or other nutrient transporter within the digestive tract to effect efficient absorption of the drug. It is believed that the active drug is subsequently released from the amino acid or peptide prodrug by hepatic and extrahepatic hydrolases that are in part, present in plasma.

Improvement in the pharmacokinetics of the compounds described herein is another advantage of the prodrugs of the present invention. Administration of such a prodrug could not only result in more efficient absorption of normally poorly absorbed compounds but also lead to maintenance of plasma drug levels as the result of continuing generation of the active drug from a plasma reservoir of the prodrug.

Uses and Methods of the Invention

One embodiment of the present invention is a method of treating a disorder in a subject in need thereof. The method comprises orally administering a therapeutically effective amount of a prodrug of the present invention to the subject, or a pharmaceutically acceptable salt thereof. The disorder may be one treatable with the drug.

The prodrugs encompassed by the present invention may be administered in conjunction with other therapies and/or in combination with other complementary active agents. In such combination therapies, the prodrugs encompassed by the present invention may be administered prior to, concurrent with, or subsequent to the other therapy and/or active agent. The prodrug and other active agent(s) may also be incorporated into a single dosage form.

In one embodiment, the present invention is directed to a method for minimizing the gastrointestinal side effects normally associated with administration of a drug, wherein the drug has a derivatisable group. The method comprises orally administering a prodrug or a pharmaceutically acceptable salt thereof to a subject in need thereof, and wherein upon oral administration, the prodrug or pharmaceutically acceptable salt minimizes, if not completely avoids, the gastrointestinal side effects usually seen after oral administration of the unbound drug. The amount of the drug is preferably a therapeutically effective amount. According to one preferred embodiment, the prodrug includes the same active as the discontinued drug. In this embodiment, the prodrug can be any prodrug of Formula I or a pharmaceutically acceptable salt thereof. In a further embodiment, the prodrug can be selected from any succinyl-valine ester or glutaryl para-amino benzoic acid ester presented herein wherein the succinyl and glutaryl portions are bound to the derivatisable group on the drug.

In yet another embodiment, the present invention is directed to a method for improving the efficiency of drug absorption compared to that after administering the non-conjugated drug. The method comprises administering to a subject in need thereof, a prodrug or a pharmaceutically acceptable salt thereof, and wherein upon oral administration plasma drug levels are at least 20% higher than when the underivatised drug is administered. The amount of the drug is preferably a therapeutically effective amount. In this embodiment, the prodrug can be any prodrug of Formula I or a pharmaceutically acceptable salt thereof. In a further embodiment, the prodrug can be selected from any succinyl-valine ester or glutaryl para-amino benzoic acid ester presented herein.

In another embodiment, the present invention is directed to a method for sustaining plasma drug levels for a significantly longer period than when the non-conjugated drug is administered alone. The method comprises administering, to a subject in need thereof, a prodrug or a pharmaceutically acceptable salt thereof to a subject in need thereof, and wherein upon oral administration, plasma drug levels are sustained for at least 50% longer than when the underivatised drug is administered. The amount of the drug is preferably a therapeutically effective amount. In this embodiment, the prodrug can be any prodrug of Formula I or a pharmaceutically acceptable salt thereof. In a further embodiment, the prodrug can be selected from any succinyl-valine ester or glutaryl para-amino benzoic acid ester presented herein.

Salts, Solvates, and Derivatives of the Compounds of the Invention

The compounds, compositions and methods of the present invention further encompass the use of salts and solvates of the prodrugs described herein. In one embodiment, the invention disclosed herein is meant to encompass all pharmaceutically acceptable salts of prodrugs (including those of the carboxyl terminus of the amino acid as well as those of any basic nitrogen).

Typically, a pharmaceutically acceptable salt of a prodrug of the present invention is prepared by reaction of the prodrug with a desired acid or base, as appropriate. The salt may precipitate from solution and be collected by filtration or may be recovered by evaporation of the solvent. For example, an aqueous solution of an acid such as hydrochloric acid may be added to an aqueous suspension of the prodrug and the resulting mixture evaporated to dryness (lyophilized) to obtain the acid addition salt as a solid. Alternatively, the prodrug may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention may have both a basic and an acidic centre and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain (1999). Polymorphism in Pharmaceutical solids. Marcel Decker, New York). The compounds useful in the practice of the present invention can have one or more chiral centers and, depending on the nature of individual substituents, they can also have geometrical isomers.

Pharmaceutical Compositions of the Invention

While it is possible that, for use in the methods of the invention, the prodrug of the present invention may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice. In one embodiment of the present invention, a composition comprising a prodrug of the present invention (e.g., a prodrug of any of Formulae 1-x) is provided. The composition comprises at least one prodrug selected from Formula 1-x, and at least one pharmaceutically acceptable excipient or carrier.

The formulations of the invention may be immediate—release dosage forms, i.e., dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, e.g., in Remington, The Science and Practice of Pharmacy, 20^{th} Edition, 2000, pp. 858-914.

However since absorption of amino acid and peptide pro-drugs may proceed via an active transporter such as Pept1, controlled release dosage forms may be desirable. As the Pept1 transporter is believed to be largely confined to the upper GI tract this may limit the opportunity for continued absorption along the whole length of the GI tract.

For those prodrugs which do not result in sustained plasma drugs levels due to continuous generation of active agent from a plasma reservoir of prodrug—but which may offer other advantages—gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to retain drug in the stomach, slowing drug passage into the ileum maximizing the period over which absorption take place and effectively prolonging plasma drug levels. Other drug delivery systems affording delayed progression along the GI tract may also be of value.

The formulations of the present invention can be administered from one to six times daily, depending on the dosage form and dosage.

In one embodiment, the present invention provides a pharmaceutical composition comprising at least one active pharmaceutical ingredient (i.e., a prodrug), or a pharmaceutically acceptable derivative (e.g., a salt or solvate) thereof, and a pharmaceutically acceptable carrier or excipient. In particular, the invention provides a pharmaceutical composition comprising a therapeutically effective amount of at least one prodrug of the present invention, or a pharmaceutically acceptable derivative thereof, and a pharmaceutically acceptable carrier or excipient.

The prodrug employed in the present invention may be used in combination with other therapies and/or active agents. Accordingly, the present invention provides, in another embodiment, a pharmaceutical composition comprising at least one compound useful in the practice of the present invention, or a pharmaceutically acceptable salt or solvate thereof, a second active agent, and, optionally a pharmaceutically acceptable carrier or excipient.

When combined in the same formulation, it will be appreciated that the two compounds are preferably stable in the presence of, and compatible with each other and the other components of the formulation. When formulated separately, they may be provided in any convenient formulation, conveniently in such manner as are known for such compounds in the art.

The prodrugs presented herein may be formulated for administration in any convenient way for use in human or veterinary medicine. The invention therefore includes pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilising agent(s).

Preservatives, stabilizers, dyes and even flavouring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, see, e.g., International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavouring and colouring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia, cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyan

The pharmaceutical compositions of the invention may contain from 0.01 to 99% weight per volume of the prodrugs encompassed by the present invention.

Dosages

The doses referred to throughout the specification refer to the amount of the drug equivalent in the particular prodrug, unless otherwise specified.

Appropriate patients to be treated according to the methods of the invention include any human or animal in need of such treatment. Methods for the diagnosis and clinical evaluation of the disease condition including its severity in an animal or human will be well known in the art. Thus, it is within the skill of the ordinary practitioner in the art (e.g., a medical doctor or veterinarian) to determine if a patient is in need of treatment. The patient is preferably a mammal, more preferably a human, but can be any subject or animal, including a laboratory animal in the context of a clinical trial, screening, or activity experiment employing an animal model. Thus, as can be readily appreciated by one of ordinary skill in the art, the methods and compositions of the present invention are particularly suited to administration to any animal or subject, particularly a mammal, and including, but not limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending on the severity of the condition to be treated, a suitable therapeutically effective and safe dosage, as may readily be determined within the skill of the art, can be administered to subjects. For oral administration to humans, the daily dosage level of the prodrug may be in single or divided doses. The duration of treatment may be determined by one of ordinary skill in the art, and should reflect the nature of the condition and/or the rate and degree of therapeutic response to the treatment. Typically, a physician will determine the actual dosage which will be most suitable for an individual subject.

The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. For highly potent agents, the daily dose requirement may, for example, range from 0.5 to 50 mg, preferably from 1 to 25 mg, and more preferably from 1 mg to 10 mg. For less potent agents, the daily dose requirement may, for example, range from 1 mg to 1600 mg, preferably from 1 mg to 800 mg and more preferably from 1 mg to 400 mg.

In the methods of treatment, the prodrugs encompassed by the present invention may be administered in conjunction with other therapies and/or in combination with other active agents. For example, the prodrugs encompassed by the present invention may be administered to a patient in combination with other active agents used to treat that condition. An active agent to be administered in combination with the prodrugs encompassed by the present invention. In such combination therapies, the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical composition. For example, a prodrug encompassed by the present invention and another active agent may be administered in a substantially simultaneous manner, such as in a single capsule or tablet having a fixed ratio of these agents, or in multiple separate dosage forms for each agent.

When the prodrugs of the present invention are used in combination with another agent active in the methods for treating that condition, the dose of each compound may differ from that when the compound is used alone. Appropriate doses will be readily appreciated by those of ordinary skill in the art.

### EXAMPLES

The present invention is further illustrated by reference to the following Examples. However, it should be noted that these Examples, like the embodiments described above, are illustrative and are not to be construed as restricting the enabled scope of the invention in any way.

Preparation of the Prodrugs of the Present Invention

Compounds employed in the present invention may be prepared by the general methods provided herein.

### Example 1a

### General Route of Synthesis for Amino Acid or Peptide Dicarboxylic Acid Conjugates

Three general routes of synthesis of prodrugs of various drugs as their HCl or TFA salts are given in Scheme 1 (alcohol ester), Scheme 2 (enol ester) and Scheme 3 (amine amide) below. These routes of synthesis are illustrated using a succinic acid linker. This can, however, be applied to all linkers of the present invention.

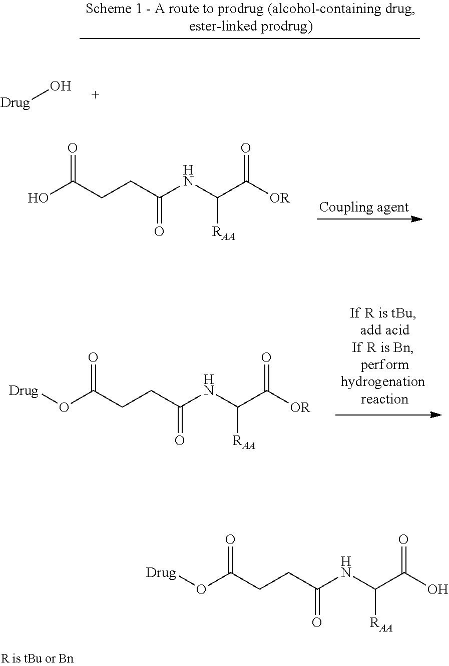

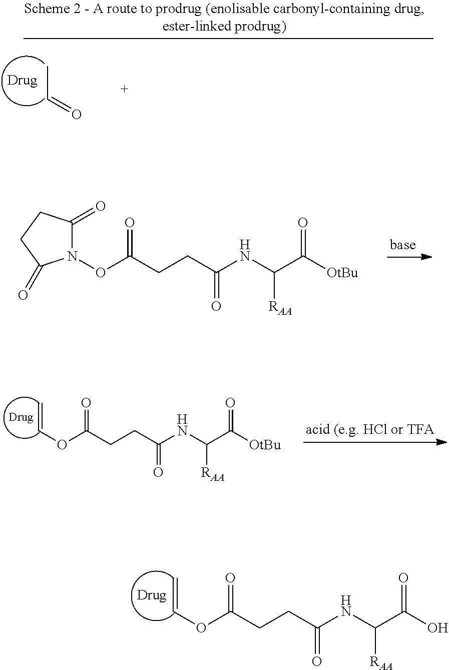

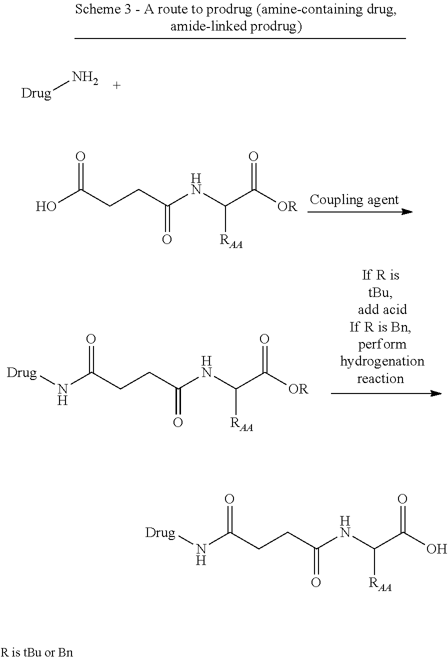

### Example 1b

### General Route of Synthesis of Dicarboxylate Conjugates

Three general routes of synthesis of prodrugs of various drugs are given in Scheme 4a (alcohol ester), Scheme 4b (enol ester) and Scheme 4c (amine amide) below. These routes of synthesis are illustrated using a succinic acid linker. This can, however, be applied to all linkers of the present invention.

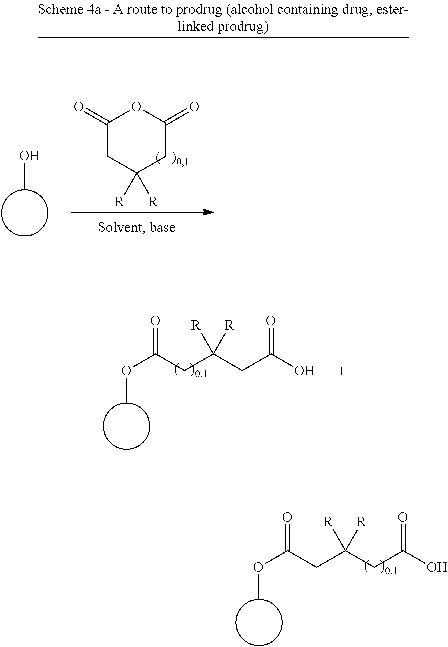

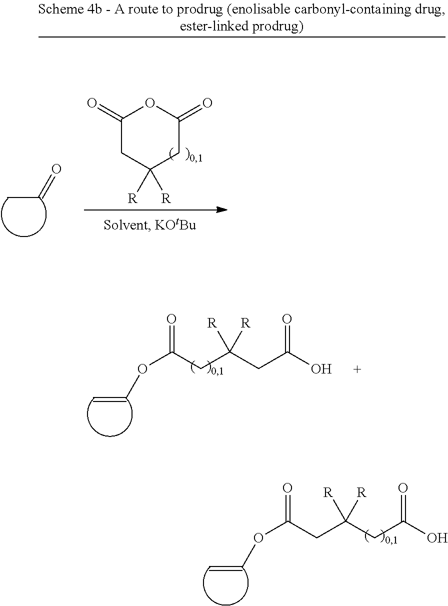

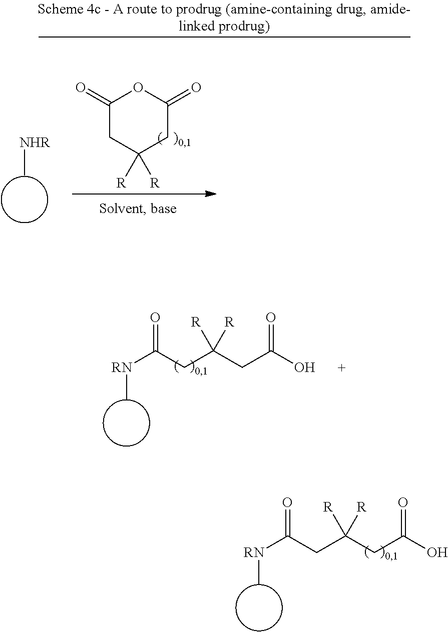

### Example 2

### Synthesis of metronidazole-[succinyl-(S)-valine]ester

Metronidazole-[succinyl-(S)-valine]ester was prepared from (S)-valine tert-butyl ester hydrochloride in three steps (see Scheme 5).

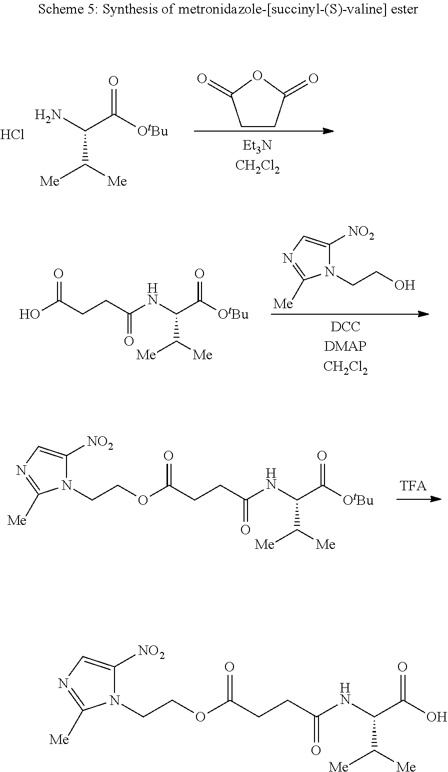

(S)-Valine tert-butyl ester hydrochloride was treated with succinic anhydride in the presence of triethylamine to give succinyl-(S)-valine tert-butyl ester which was then coupled to metronidazole via a N,N-dicyclohexylcarbodi-imide (DCC) mediated reaction to give metronidazole-[succinyl-(S)-valine tert-butyl ester]. After purification, the tert-butyl ester was cleaved by treatment with trifluoroacetic acid to give metronidazole-[succinyl-(S)-valine]ester free base in >95% purity by HPLC and NMR.

### Detail

To a stirred solution of (S)-valine tert-butyl ester hydrochloride (5.00 g, 23.8 mmol) in anhydrous dichloromethane (125 mL) was added triethylamine (5.31 g, 7.31 mL, 52.5 mmol) and succinic anhydride (2.63 g, 26.3 mmol) and the reaction was stirred at room temperature overnight. The solution was diluted with dichloromethane (100 mL) and washed with 5% citric acid (2×100 mL), brine (100 mL), dried (MgSO₄) and concentrated to give succinyl-(S)-valine tert-butyl ester (6.4 g, 98%), as a white solid.

To a stirred solution of metronidazole (0.50 g, 2.92 mmol) in anhydrous dichloromethane (30 mL) was added succinyl-(S)-valine tert-butyl ester (1.04 g, 3.80 mmol), 4-dimethylaminopyridine (7 mg, 0.058 mmol), N,N-dicyclohexylcarbodi-imide (0.84 g, 4.09 mmol) and the reaction was stirred at room temperature overnight. The resulting suspension was filtered through Celite and the filtrate concentrated to afford an oil which was purified by medium-pressure chromatography on silica eluting with a gradient of 0→5% methanol in dichloromethane to afford metronidazole-[succinyl-(S)-valine tert-butyl ester] (1.54 g) which was further purified using a Biotage Isolera automated chromatography system under normal phase conditions (silica column, gradient of 15→100% ethyl acetate in petrol) with detection at 313 nm to afford metronidazole-[succinyl-(S)-valine tert-butyl ester] (1.07 g, 86%), as a clear oil. R_{f} 0.50 (methanol-dichloromethane, 1:9 v/v).

Metronidazole-[succinyl-(S)-valine tert-butyl ester] (1.03 g, 2.42 mmol) in trifluoroacetic acid (20 mL) was stirred at room temperature for 45 min. The resulting solution was evaporated to dryness and residual trifluoroacetic acid was removed azeotropically with chloroform (5×35 mL) to afford metronidazol-[succinyl-(S)-valine]ester which was purified using a a Biotage Isolera automated chromatography system under reversed-phase conditions (C₁₈ column, gradient of 0→100% acetonitrile in 0.02% hydrochloric acid) with detection at 279 nm to afford, after freeze-drying, metronidazole-[succinyl-(S)-valine]ester (0.39 g, 43%), as a tan coloured solid.

### Example 3

### Synthesis of metronidazole-(glutaryl-para-amino benzoic acid [PABA]) ester

Metronidazole-(glutaryl-PABA) ester was synthesised from PABA tert-butyl ester in three reaction steps (see Scheme 6).

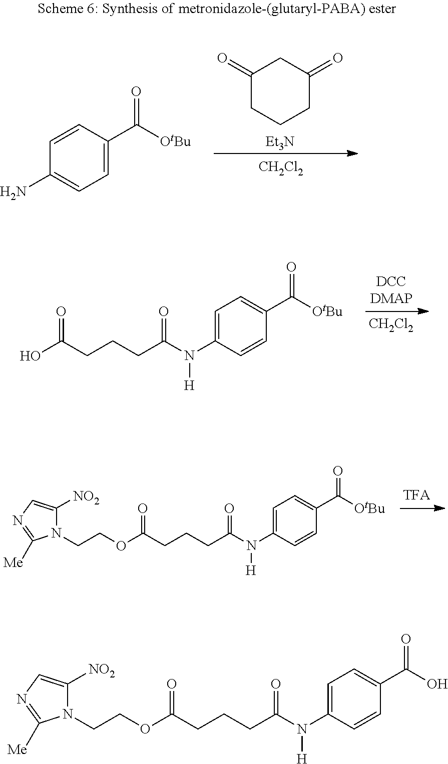

PABA tert-butyl ester was treated glutaric anhydride in the presence of triethylamine to give glutaryl-PABA tert-butyl ester which was then coupled to metronidazole using N,N-dicyclohexylcarbodi-imide to give metronidazole-(glutaryl-PABA tert-butyl ester). After purification, the tert-butyl ester was cleaved using trifluoroacetic acid to give the required metronidazole-(glutaryl-PABA) ester free base in >95% purity by HPLC and NMR.

### Detail

To a stirred solution of 4-aminobenzoic acid (PABA) tert-butyl ester (2.50 g, 12.9 mmol) in ethyl acetate (125 mL) was added triethylamine (1.70 g, 2.35 mL, 16.8 mmol) and glutaric anhydride (2.21 g, 19.4 mmol) and the reaction was stirred at room temperature overnight. The solution was washed with 5% citric acid (3×75 mL), water (75 mL), brine (100 mL), dried (MgSO₄) and concentrated to give glutaryl-PABA tert-butyl ester (3.84 g, 97%), as a white solid.

To a stirred solution of metronidazole (0.50 g, 2.92 mmol) in anhydrous dichloromethane (30 mL) was added glutaryl-PABA tert-butyl ester (1.17 g, 3.80 mmol), 4-dimethylaminopyridine (8 mg, 0.058 mmol), N,N-dicyclohexylcarbodi-imide (0.84 g, 4.09 mmol) and the reaction was stirred at room temperature overnight. The resulting suspension was filtered through Celite and the filtrate concentrated to an oil which was purified by medium-pressure chromatography on silica eluting with a gradient of 40→100% ethyl acetate in petrol to afford metronidazole-(glutaryl-PABA tert-butyl ester) (1.16 g, 86%), as a clear oil. R_{f} 0.45 (ethyl acetate-petrol, 1:1 v/v).

Metronidazole-(gluratyl-PABA tert-butyl ester) (1.10 g, 2.39 mmol) in trifluoroacetic acid (22 mL) was stirred at room temperature for 45 min. The resulting solution was evaporated to dryness and residual trifluoroacetic acid was removed azeotropically with chloroform (5×40 mL) to afford metronidazole-(glutaryl-PABA) ester (0.95 g, 98%), as an off-white solid.

### Example 4

### Assessment of Chemical/Biological Stability of Metronidazole Prodrugs

To avoid direct interaction between the active drug and the gut microfloral population, the prodrug should not undergo premature chemical or enzymic cleavage back to the parent dug while within the gut lumen. To confirm this the prodrugs were incubated with USP SGF (simulated gastric fluid), FeSSIF (fed state simulated intestinal fluid) and FaSSIF (fasted state simulated intestinal fluid) and remaining prodrug and any generated active drug assayed by use of a qualified LC-MS/MS analytical method.

Method—Prodrug (20 μg/mL) in the FaSSIF cocktail comprising sodium taurocholate 3 mM, lecithin 0.75 mM, NaOH 8.7 mM, NaH₂PO₄. H₂O 28.7 mM, NaCl 106 mM, pancreatin 1% (overall pH 6.5) was incubated for 2 h at 37° C. After acidifying the incubation to stop any reaction the amount of prodrug remaining and drug generated were determined using a qualified LC-MS/MS method.

The corresponding FeSSIF cocktail, which was used in comparable manner, comprised sodium taurocholate 15 mM, lecithin 3.75 mM, NaOH 101 mM, acetic acid 144 mM, and NaCl 202 mM (overall pH 5.0). Pancreatin was again added at 1%.

The USP simulated gastric juice comprised NaCl 34 mM, HCl ˜70 nM, pepsin 0.32% (overall pH1.1) and was incubated with prodrug (20 μg/mL) for 1 h at 37° C. Remaining prodrug and any generated active drug were assayed using a qualified LC-MS/MS method.

Results—The results shown in Table 2 indicate that both metronidazole prodrugs were chemically and enzymically stable under the conditions likely to prevail the GI tract. As such there should minimal potential for direct interaction between active drug and the gut microfloral population.

TABLE 2
Stability of metronidazole succinyl valine ester and metronidazole
glutaryl PABA ester in simulated gastric and intestinal juice

| | | Simululated | Simululated |
|---|---|---|---|
| | Simulated | intestinal juice | intestinal juice |
| | gastric juice | (2 h, 37° C.) | (2 h, 37° C.) |
| | (1 h, 37° C.) | (FaSSIF) | (FeSSIF) |
| | % active | % active | % active |
| | drug | drug | drug |
| Compound | generated | generated | generated |
| Metronidazole | <1% | <1% | <1% |
| succinyl valine | | | |
| ester | | | |
| Metronidazole | 1% | 2% | <1% |
| glutaryl PABA | | | |
| ester | | | |

### Example 5

### Assessment of the Antimicrobial Activity of Prodrugs of Metronidazole

Prodrugging which results in transient chemical inactivation of antibiotics should serve to avoid disturbance of the normal microbial balance in the human gut. It is well known that overgrowth of C. difficile resulting from such disturbance is associated with profound diarrhoea and dehydration. Introduction of a bulky amino acid substituent into an antibiotic structure would likely inactivate the drug. Consequently a prototypic antibiotic, metronidazole, was chosen for investigation and derivatized in this way.

Methods—The Minimum Inhibitory Concentrations (MIC) of two representative metronidazole pro-drugs (compared with metronidazole itself) were determined against a range of anaerobic bacteria. The MIC values were established using an agar dilution procedure based on procedures described by Clinical and Laboratory Standards Institute (CLSI). (Methods for Antimicrobial Susceptibility Testing of Anaerobic Bacteria; Approved Standard-Seventh Edition. CLSI Document M11-A7. Wayne, Pa. CLSI; 2007).

Activity against some 31 different anaerobic bacteria were tested. The concentration range tested was from 0.03 mg/L to 64 mg/L. Each test compound was prepared in molten agar, allowed to set and then innoculated with the strains of bacteria described above and incubated at 37° C. for 24-72 h under anaerobic conditions. The plates were subsequently examined and the endpoint MIC for each test organism recorded as the lowest concentration at which no growth was evident

Results—The results are presented in Table 3 and show a dramatic reduction in anti-microbial activity of these prodrugs compared to the parent drug. This should ensure that while resident in the gut lumen these prodrugs are unlikely to alter the delicate microbial balance.

TABLE 3
Minimum inhibitory concentration against anaerobic bacteria of
metronidazole-succinyl-S-valine ester and metronidazole-glutaryl-PABA
ester and metronidazole

| | MIC - μg/ml | | |
|---|---|---|---|
| | | | M- |
| | | M-succinyl | glutaryl |
| Test organism | Metronidazole | ester | ester |
| Bacteroides fragilis - ATCC | 0.5 | >64 | >64 |
| 25285 | | | |
| Bacteroides fragilis - clinical | 0.5 | >64 | >64 |
| isolate | | | |
| Bacteroides thetaiotamicron - | 1 | >64 | >64 |
| ATCC 29741 | | | |
| Bacteroides thetaiotamicron - | 1 | >64 | >64 |
| NCTC 10582 | | | |
| Bifidobacterium adolescentis - | 32 | >64 | >64 |
| NCTC 11814 | | | |
| Bifidobacterium dentium - | 16 | >64 | >64 |
| NCTC 11816 | | | |
| Bifidobacterium longum - | 8 | >64 | >64 |
| NCTC 11818 | | | |
| Clostridium bifermentans - | 0.25 | >64 | 64 |
| ATCC 638 | | | |
| Clostridium difficile - ATCC | 0.12 | 32 | 8 |
| 700057 | | | |
| Clostridium difficile - | 0.06 | 32 | 8 |
| antibiotic-susceptible clinical | | | |
| isolate | | | |
| Clostridium difficile - NCTC | 0.12 | 64 | 16 |
| 13366 - NAP1/027 | | | |
| Clostridium novyi - ATCC | 0.5 | >64 | 32 |
| 19402 | | | |
| Clostridium perfringens - | 1 | >64 | >64 |
| ATCC 13124 | | | |
| Clostridium perfringens - | 2 | >64 | >64 |
| antibiotic-susceptible clinical | | | |
| isolate | | | |
| Clostridium sordellii - NCTC | 0.5 | >64 | 64 |
| 13356 | | | |
| Clostridium sporogenes - | 0.06 | 32 | 8 |
| ATCC 3584 | | | |
| Eggerthella lenta - ATCC | 0.5 | >64 | 32 |
| 43055 | | | |
| Fusobacterium necrophorum - | 0.12 | 32 | 8 |
| clinical isolate | | | |
| Peptostreptococcus | 0.12 | 32 | 16 |
| anaerobius - NCTC 11460 | | | |
| Peptostreptococcus magnus - | 0.25 | 64 | 16 |
| clinical isolate | | | |
| Peptostreptococcus micros - | 0.25 | 64 | 8 |
| clinical isolate | | | |
| Porphyromonas | 0.5 | >64 | 8 |
| assacharolyticus - clinical | | | |
| isolate | | | |
| Porphyromonas endodontalis - | 1 | >64 | 4 |
| clinical isolate | | | |
| Prevotella bivia - ATCC | 1 | >64 | 32 |
| 29303 | | | |
| Prevotella denticola - clinical | 0.5 | >64 | 64 |
| isolate | | | |
| Propionibacterium acnes - | >64 | >64 | >64 |
| clinical isolate MIC | | | |
| clindamycin 0.12 mg/L | | | |
| Propionibacterium acnes - | >64 | >64 | >64 |
| clinical isolate MIC | | | |
| clindamycin 0.5 mg/L | | | |
| Veillonella atypical - NCTC | 8 | >64 | >64 |
| 11830 | | | |
| Veillonella parvula - NCTC | 4 | >64 | >64 |
| 11810 | | | |
| Veillonella spp.- clinical | 2 | >64 | >64 |
| isolate | | | |
| Campylobacter coli - ATCC | 0.5 | >64 | 64 |
| 33559 | | | |
| Campylobacter coli - clinical | 1 | >64 | >64 |
| isolate | | | |
| Campylobacter jejuni - ATCC | 1 | >64 | 64 |
| 33560 | | | |
| Campylobacter jejuni - | 8 | >64 | >64 |
| clinical isolate | | | |
| Helicobacter pylori - clinical | 2 | 64 | 32 |
| isolate | | | |
| Helicobacter pylori - clinical | 2 | 64 | 16 |
| isolate | | | |

### Example 6

### Bioavailability of Metronidazole from its Succinyl Valine and Glutaryl Para-Amino Benzoic Acid Ester Prodrugs in the Rat

In order to confirm successful absorption and subsequent hydrolysis of these metronidazole prodrugs, a comparative bioavailability study was undertaken in the rat Methods—Three groups of five male Sprague Dawley rats were orally dosed with either metronidazole itself or its succinyl valine or glutaryl para-amino benzoic acid ester prodrugs. Serial tail vein blood samples were subsequently collected via an indwelling cannuala into EDTA tubes containing a stabilising cocktail (including 6 mg/mL NaF) to prevent post collection enzymic hydrolysis of the prodrug. Separated plasma samples were subjected to bioanalysis for both the prodrugs and metronidazole itself using a qualified LC/MS-MS bioanalytical method. Subsequent pharmacokinetic analysis was then undertaken using Win NonLin to generate the relevant PK parameters including AUC and T_{1/2} & T_{50%}Cₘₐₓ the period for which plasma drug conentrations remained at or above 50% of the Cₘₐₓ

Results—These are shown in Table 4, 5 & 6.

TABLE 4
Pharmacokinetics of metronidazole after oral dosing of 1
mg metronidazole free base equivalents/kg to male rats

| Pharmacokinetic | | | | | | | |
|---|---|---|---|---|---|---|---|
| parameter | 1 | 2 | 3 | 4 | 5 | Mean | sd |
| Cₘₐₓ (ng/mL) | 1510 | 1110 | 1050 | 1050 | 1200 | 1180 | 192 |
| Tₘₐₓ (h) | 0.25 | 0.25 | 0.25 | 0.25 | 0.50 | 0.3 | — |
| AUC (ng · h/mL) | 4140 | 2620 | 3400 | 3670 | 4020 | 3570 | 605 |
| t½ (h) | 2.1 | 1.6 | 2.0 | 1.8 | 2.0 | 1.9 | 0.19 |
| T_{>50% Cmax} (h) | 1.5 | 1.75 | 2.75 | 3.0 | 2.5 | 2.3 | 0.65 |

TABLE 5
Pharmacokinetics of metronidazole after oral dosing
of 1 mg metronidazole equivs of the succinyl valine
ester prodrug equivalents/kg to male rats

| Pharmaco- | | | | | | | |
|---|---|---|---|---|---|---|---|
| kinetic | | | | | | | |
| parameter | 6 | 7 | 8 | 9 | 10 | Mean | sd |
| Cₘₐₓ (ng/mL) | 112 | 56.6 | 58.3 | 126 | 97 | 90 | 31.4 |
| Tₘₐₓ (h) | 2.0 | 2.0 | 2.0 | 2.0 | 2.0 | 2.0 | — |
| AUC | 510 | 273 | 328 | 433 | 444 | 398 | 95.5 |
| (ng · h/mL) | | | | | | | |
| t½ (h) | 1.8 | 2.3 | 1.8 | 2.4 | 2.2 | 2.1 | 0.3 |
| T_{>50% Cmax} (h) | 3.5 | 4.0 | 5.0 | 2.75 | 3.75 | 3.8 | 0.82 |
| Fᵣₑₗ (%) | 12.3 | 10.4 | 9.6 | 11.8 | 11.0 | 11.02 | |

TABLE 6
Pharmacokinetics of metronidazole after oral dosing of
1 mg metronnidazole metronidazole equivs of the glutaryl
PABA ester prodrug equivalents/kg to male rats

| Pharmaco- | | | | | | | |
|---|---|---|---|---|---|---|---|
| kinetic | | | | | | | |
| parameter | 11 | 12 | 13 | 14 | 15 | Mean | sd |
| Cₘₐₓ (ng/mL) | 78.2 | 60.3 | 80.3 | 114 | 128 | 92.2 | 27.9 |
| Tₘₐₓ (h) | 2.0 | 2.0 | 2.0 | 2.0 | 2.0 | 2.0 | — |
| AUC | 410 | 323 | 316 | 661 | 836 | 509 | 230 |
| (ng · h/mL) | | | | | | | |
| t½ (h) | 2.0 | 1.9 | 1.6 | 2.0 | 2.0 | 1.9 | 0.18 |
| T_{>50% Cmax} (h) | 4.5 | 5.0 | 3.25 | 5.25 | 6.0 | 4.8 | 1.02 |
| Fᵣₑₗ (%) | 10.0 | 12.3 | 9.3 | 18.0 | 20.8 | 14.0 | |

Following administration of metronidazole itself, peak plasma levels were seen very rapidly—within 15 mins of dosing. Subsequent elimination also took place rapidly with a half-life of −2 h. By contrast metronidazole plasma levels after either prodrug did not peak until around 2 h post dosing and, although significantly lower than after the parent drug, persisted for several hours. Thus the mean T_{50%}Cₘₐₓ (the period for which plasma metronidazole levels stayed at or above 50% of the maximum concentration) after the succinyl valine ester was 3.8±0.82 h compared with 2.3±0.65 h after giving metronidazole itself. Similarly this value, after the glutaryl PABA ester, was 4.8±1.02 h, some two-fold longer than that after administration of the parent drug. The relative bioavailability after these prodrugs was 11-14% of that seen after giving the drug itself. Prodrug plasma levels were low relative to those of the generated parent drug (<0.66-1.3%) and disappeared very quickly (within 2 h).

These result confirm the potential of these prodrugs to deliver the active drug to the systemic circulation but importantly also confer the advantage of prolonged plasma drug concentrations. If this translates to man it should mean less frequent dosing and increased patient compliance.

### Example 7

### Synthesis of Clindamycin Prodrugs

Clindamycin is a valuable antibiotic especially useful in the treatment of multidrug-resistant Staphylococcus aureus (MRSA) infections. Few antibiotics have been found to be effective in MRSA and, although clindamycin is one such agent, regrettably its use is frequently marred by adverse GI events.

Transient inactivation of clindamycin by synthesis of its dicarboxyate amino acid prodrug should enable drug to be delivered to the systemic circulation while minimising the direct adverse effects on the gut. Subsequent to absorption plasma and liver esterase activity would release the active drug.

Clindamycin succinyl valine ester

Clindamycin-2-[succinyl-(S)-valine]ester trifluoroacetate was synthesised from clindamycin using a 3-step procedure shown in the scheme 7 below:

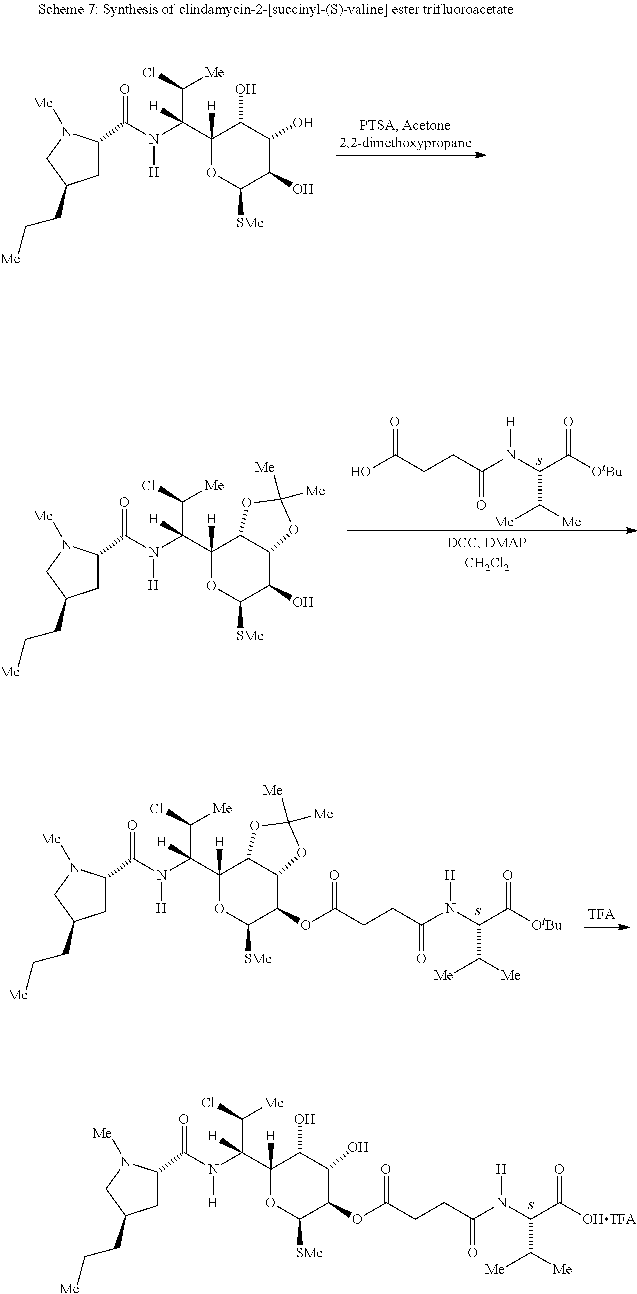

Acetal protection of the 3,4-hydroxyl groups of clindamycin afforded 3,4-acetal protected clindamycin which was subsequently coupled with succinyl-(S)-valine tert-butyl ester via an N,N-dicyclohexylcarbodi-imide (DCC) mediated reaction to give 3,4-acetal-protected clindamycin-2-[succinyl-(S)-valine tert-butyl ester]ester. The tert-butyl ester and acetal groups were cleaved using trifluoroacetic acid to give clindamycin-2-[succinyl-(S)-valine]ester trifluoroacetate as a white solid, after reversed-phase purification.

### DETAILED DESCRIPTION

To a solution of clindamycin (0.70 g, 1.65 mmol) in 2,2-dimethoxypropane (10 mL) was added a solution of para-toluenesulfonic acid (0.42 g, 2.22 mmol) in acetone (10 mL) and the mixture was stirred for 3 h. The resulting mixture was concentrated and the residue partitioned between ethyl acetate (50 mL) and saturated aqueous sodium bicarbonate (50 mL). The organic layer was washed with aqueous sodium bicarbonate (50 mL), brine (50 mL), dried (MgSO₄) and concentrated to afford 3,4-acetal-protected clindamycin (0.68 g, 88%), as a yellow oil.

To a stirred solution of 3,4-acetal-protected clindamycin (0.68 g, 1.46 mmol), succinyl-(S)-valine tert-butyl ester (0.52 g, 1.90 mmol) and 4-dimethylaminopyridine (4 mg, 0.03 mmol) in dichloromethane (15 mL) was added N,N′-dicyclohexylcarbodi-imide (0.42 g, 2.05 mmol) in one portion and stirring was continued overnight. The resulting suspension was filtered through Celite and the filtrate was concentrated to a yellow oil (1.1 g) that was purified by medium-pressure chromatography on silica eluting with a gradient of 0→2% MeOH:NH₄OH (9:1 v/v) in dichloromethane. Further purification using a Biotage Isolera automated chromatography system under reversed-phase conditions (C₁₈ column, gradient of 0→100% MeCN in 0.02% hydrochloric acid) with detection at 210 nm afforded 3,4-acetal-protected clindamycin-2-[succinyl-(S)-valine tert-butyl ester]ester hydrochloride (0.29 g, 27%), as an off-white solid. R_{f} 0.50 [95% dichloromethane-5% (MeOH:NH₄OH, 9:1 v/v)].

3,4-Acetal-protected clindamycin-2-[succinyl-(S)-valine tert-butyl ester]ester hydrochloride (0.29 g, 0.40 mmol) in trifluoroacetic acid (6 mL) was stirred at room temperature for 45 min. The mixture was evaporated to dryness and residual trifluoroacetic acid was removed azeotropically with chloroform (5×20 mL). The resulting solid was purified using a Biotage Isolera automated chromatography system under reversed-phase conditions (C₁₈ column, gradient of 0→100% MeCN in 0.1% aqueous trifluoroacetic acid) with detection at 210 nm to afford, after freeze-drying, clindamycin-2-[succinyl-(S)-valine]ester trifluoroacetate (0.17 g, 57%), as a white solid.

¹H NMR (DMSO-d₆) Spectrum

12.61 (br s, 1H, CO₂H), 9.73 (br s, 1H, NH⁺), 8.62 (d, J=8.9 Hz, 1H, NH), 8.09 (d, J=8.6 Hz, 1H, NH), 5.40 (d, J=5.6 Hz, 1H, SCH) 5.29 (br s, 1H, OH), 5.10-5.00 (m, 2H, OH+CH), 4.57-4.51 (m, 2H, 2×CH), 4.17-4.07 (m, 3H, α-CH+2×CH), 3.79-3.43 (m, 7H, CH+3×CH₂), 2.94-2.78 (m, 4H, NCH₃+CH), 2.27-2.14 (m, 2H, CH₂), 2.08-1.98 (m, 6H, SCH₃+CH₂+β-CH), 1.46-1.23 (m, 8H, CH₃+2×CH₂+CH), 0.90-0.86 (m, 9H, 3×CH₃).

Clindamycin Glutaryl PABA Ester

Clindamycin-2-(glutaryl-PABA) ester trifluoroacetate was synthesised from clindamycin by a procedure involving 3 reaction steps shown in the scheme 8 below.

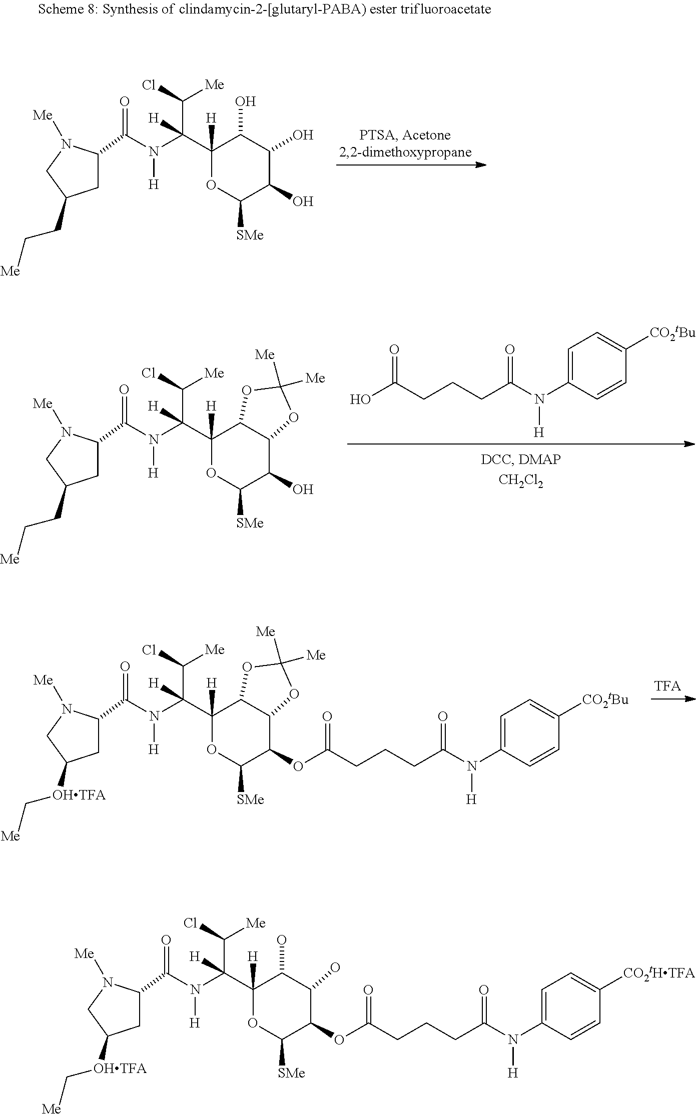

Acetal protection of the 3,4-hydroxyl groups afforded 3,4-acetal protected clindamycin which was subsequently coupled with glutaryl-PABA tert-butyl ester via an N,N-dicyclohexylcarbodi-imide (DCC) mediated reaction to give 3,4-acetal-protected clindamycin-2-(glutaryl-PABA tert-butyl ester) ester. The tert-butyl ester and acetal groups were simultaneously cleaved using trifluoroacetic acid to give clindamycin-2-(glutaryl-PABA) ester trifluoroacetate as a white solid, after reversed-phase purification.

### DETAILED DESCRIPTION

To a solution of clindamycin (0.45 g, 1.06 mmol) in 2,2-dimethoxypropane (11 mL) was added a solution of para-toluenesulfonic acid (0.27 g, 1.43 mmol) in acetone (2.5 mL) and the mixture was stirred for 3 h. The resulting mixture was concentrated and the residue partitioned between ethyl acetate (50 mL) and saturated aqueous sodium bicarbonate (50 mL). The organic layer was washed with aqueous sodium bicarbonate (50 mL), brine (50 mL), dried (MgSO₄) and concentrated to afford 3,4-acetal-protected clindamycin (0.57 g), as a yellow oil which was used without further purification.

To a stirred solution of 3,4-acetal-protected clindamycin (0.50 g, 1.08 mmol), glutaryl-PABA tert-butyl ester (0.43 g, 1.40 mmol) and 4-dimethylaminopyridine (3 mg, 0.02 mmol) in dichloromethane (11 mL) was added N,N-dicyclohexylcarbodi-imide (0.31 g, 1.51 mmol) in one portion, and stirring was continued overnight. The resulting suspension was filtered through Celite and the filtrate was concentrated to give a yellow oil that was purified by medium-pressure chromatography on silica eluting with a gradient of 0→2% MeOH:NH₄OH (9:1 v/v) in dichloromethane to afford 3,4-acetal-protected clindamycin-2-(glutaryl-PABA tert-butyl ester) ester (0.86 g), as an off-white solid that was used without further purification.

R_{f} 0.55 [95% dichloromethane-5% (MeOH:NH₄OH, 9:1 v/v)].

3,4-Acetal-protected clindamycin-2-(glutaryl-PABA tert-butyl ester) ester (0.86 g, 1.14 mmol) in trifluoroacetic acid (17 mL) was stirred at room temperature for 45 min. The mixture was evaporated to dryness and residual trifluoroacetic acid was removed azeotropically with chloroform (5×20 mL). The resulting solid was purified using a Biotage Isolera automated chromatography system under reversed-phase conditions (C₁₈ column, gradient of 0→100% MeCN in 0.1% aqueous trifluoroacetic acid) with detection at 269 nm to afford, after freeze-drying, clindamycin-2-(glutaryl-PABA) ester trifluoroacetate (0.51 g, 62% over three steps), as a white solid.

¹H NMR (DMSO-d₆) Spectrum

12.70 (br s, 1H, CO₂H), 10.23 (s, 1H, NH), 9.73 (br s, 1H, NH⁺), 8.62 (d, J=8.9 Hz, 1H, NH), 7.87 (d, J=8.8 Hz, 2H, 2×ArH), 7.69 (d, J=8.8 Hz, 2H, 2×ArH), 5.46 (d, J=5.6 Hz, 1H, SCH) 5.06-5.02 (m, 1H, CH), 4.55-4.41 (m, 2H, 2×CH), 4.14-4.04 (m, 2H, 2×CH), 3.82-3.86 (m, 1H, CH), 3.68-3.57 (m, 2H, CH+0.5×CH₂), 2.94-2.77 (m, 4H, NCH₃+0.5×CH₂), 2.47-2.34 (m, 4H, 2×CH₂), 2.33-2.12 (m, 2H, CH₂), 2.07 (s, 4H, SCH₃, CH), 1.92-1.80 (m, 2H, CH₂), 1.44-1.13 (m, 7H, CH₃+2×CH₂), 0.88 (t, J=7.1 Hz, 3H, CH₃).

### Example 8

### Assessment of Chemical/Biological Stability of Clindamycin Prodrugs

To avoid direct interaction between the active drug and the gut microfloral population, the prodrug should not undergo premature chemical or enzymic cleavage back to the parent dug while within the gut lumen. To confirm this the prodrugs were incubated with USP SGF (simulated gastric fluid), FeSSIF (fed state simulated intestinal fluid) and FaSSIF (fasted state simulated intestinal fluid) and remaining prodrug and any generated active drug assayed by use of a qualified LC-MS/MS analytical method.

Method—Prodrug (20 μg/mL) in the FaSSIF cocktail comprising sodium taurocholate 3 mM, lecithin 0.75 mM, NaOH 8.7 mM, NaH₂PO₄. H₂O 28.7 mM, NaCl 106 mM, pancreatin 1% (overall pH 6.5) was incubated for 2 h at 37° C. After acidifying the incubation to stop any reaction the amount of prodrug remaining and drug generated were determined using a qualified LC-MS/MS method.

The corresponding FeSSIF cocktail, which was used in comparable manner, comprised sodium taurocholate 15 mM, lecithin 3.75 mM, NaOH 101 mM, acetic acid 144 mM, and NaCl 202 mM (overall pH 5.0). Pancreatin was again added at 1%.

The USP simulated gastric juice comprised NaCl 34 mM, HCl ˜70 nM, pepsin 0.32% (overall pH1.1) and was incubated with prodrug (20 μg/mL) for 1 h at 37° C. Remaining prodrug and any generated active drug were assayed using a qualified LC-MS/MS method.

Results—The results shown in Table 7 indicate that both clindamycin prodrugs were chemically and enzymically stable under the conditions likely to prevail the GI tract. As such there should minimal potential for direct interaction between active drug and the gut microfloral population.

TABLE 7
Stability of clindamycin succinyl valine ester and clindamycin glutaryl
PABA ester in simulated gastric and intestinal juice

| | Simulated | Simululated | Simululated |
|---|---|---|---|
| | gastric juice | intestinal juice | intestinal juice |
| | (1 h, 37° C.) | (2 h, 37° C.) | (2 h, 37° C.) |
| | % active | (FaSSIF) | (FeSSIF) |
| | drug | % active drug | % active drug |
| Compound | generated | generated | generated |
| Clindamycin succinyl | <1% | <1% | <1% |
| valine ester | | | |
| Clindamycin glutaryl | <1% | <1% | <1% |
| PABA ester | | | |

### Example 9

### Assessment of the Antimicrobial Activity of Prodrugs of Clindamycin

Prodrugging which results in transient chemical inactivation of antibiotics should serve to avoid directly mediated adverse effects on the human gut.

Methods—The Minimum Inhibitory Concentrations (MIC) of two representative clindamycin pro-drugs (compared with clindamycin itself) were determined against a range of anaerobic bacteria using standard micobiological procedures.

Results—The results are presented in Table 8 and show a dramatic reduction in anti-microbial activity of these prodrugs compared to the parent drug. This should ensure that while resident in the gut lumen these prodrugs are unlikely to directly alter the delicate microbial balance.

TABLE 8
Minimum inhibitory concentration (ug/mL) of clindamycin-succinyl-S-valine ester and
clindmycin-glutaryl-PABA ester and clindamycin itself against various bacteria

| | Clindamycin | Clindamycin | |
|---|---|---|---|
| Bacterial isolate | prodrug 2 | prodrug 1 | Clindamycin |
| Bacteroides fragilis - ATCC 25285 | 64 | 8 | 0.5 |
| Bacteroides fragilis - clinical isolate | 16 | 8 | 0.12 |
| Bacteroides thetaiotamicron - ATCC 29741 | >64 | 16 | 2 |
| Bacteroides thetaiotamicron - NCTC 10582 | >64 | 32 | 1 |
| Bifidobacterium adolescentis - NCTC 11814 | 2 | 0.5 | ≦0.03 |
| Bifidobacterium dentium - NCTC 11816 | 2 | 0.5 | ≦0.03 |
| Bifidobacterium longum - NCTC 11818 | 2 | 0.5 | 0.12 |
| Clostridium bifermentans - ATCC 638 | 32 | 4 | 0.25 |
| Clostridium difficile - ATCC 700057 | >64 | >64 | 8 |
| Clostridium difficile - antibiotic-susceptible | >64 | >64 | 8 |
| clinical isolate | | | |
| Clostridium difficile - NCTC 13366 - NAP1/027 | >64 | 64 | 4 |
| Clostridium novyi - ATCC 19402 | 4 | 0.5 | ≦0.03 |
| Clostridium perfringens - ATCC 13124 | 8 | 1 | 0.06 |
| Clostridium perfringens - antibiotic-susceptible | 8 | 1 | 0.06 |
| clinical isolate | | | |
| Clostridium sordellii - NCTC 13356 | >64 | 16 | 0.5 |
| Clostridium sporogenes - ATCC 3584 | >64 | >64 | 32 |
| Eggerthella lenta - ATCC 43055 | 16 | 8 | 0.25 |
| Eggerthella lenta - clinical isolate | 16 | 8 | 0.25 |
| Fusobacterium necrophorum - clinical isolate | 1 | 0.12 | ≦0.03 |
| Fusobacterium nucleatum - clinical isolate | 2 | 0.25 | ≦0.03 |
| Peptostreptococcus anaerobius - NCTC 11460 | 32 | 8 | 0.25 |
| Peptostreptococcus magnus - clinical isolate | 64 | 16 | 0.5 |
| Peptostreptococcus micros - clinical isolate | 8 | 2 | 0.06 |
| Porphyromonas assacharolyticus - clinical isolate | 0.12 | 0.12 | ≦0.03 |
| Porphyromonas endodontalis - clinical isolate | 0.12 | 0.12 | ≦0.03 |
| Prevotella bivia - ATCC 29303 | 1 | 0.5 | ≦0.03 |
| Prevotella denticola - clinical isolate | 4 | 1 | ≦0.03 |
| Prevotella intermedia - clinical isolate | 0.12 | 0.12 | ≦0.03 |
| Prevotella melaninogenicus - clinical isolate | 0.5 | 0.25 | ≦0.03 |
| Prevotella melaninogenicus - clinical isolate | 1 | 0.25 | ≦0.03 |
| Prevotella oralis - clinical isolate | ≦0.03 | ≦0.03 | ≦0.03 |
| Propionibacterium acnes - clinical isolate MIC | 8 | 2 | 0.06 |
| clindamycin 0.12 mg/L | | | |
| Propionibacterium acnes - clinical isolate MIC | 8 | 2 | 0.12 |
| clindamycin 0.5 mg/L | | | |
| Veillonella atypical - NCTC 11830 | 4 | 2 | 0.06 |
| Veillonella parvula - NCTC 11810 | 4 | 1 | ≦0.03 |
| Veillonella spp.- clinical isolate | 1 | 0.5 | ≦0.03 |
| C. difficile ribotype 002 - clinical isolate | >64 | 64 | 2 |
| C. difficile ribotype 011 - clinical isolate | >64 | 64 | 4 |
| C. difficile ribotype 027 - clinical isolate | >64 | 64 | 4 |
| C. difficile ribotype 050 - clinical isolate | >64 | 32 | 4 |
| C. difficile ribotype 176 - clinical isolate | >64 | >64 | 4 |
| Campylobacter coli - ATCC 33559 | ≦0.03 | ≦0.03 | ≦0.03 |
| Campylobacter coli - clinical isolate | 0.06 | ≦0.03 | 0.06 |
| Campylobacter jejuni - ATCC 33560 | ≦0.03 | ≦0.03 | ≦0.03 |
| Campylobacter jejuni - clinical isolate | ≦0.03 | ≦0.03 | ≦0.03 |
| Helicobacter pylori - clinical isolate | ≦0.03 | ≦0.03 | ≦0.03 |
| Helicobacter pylori - clinical isolate | ≦0.03 | ≦0.03 | ≦0.03 |
| Staphylococcusaureus ATCC 29213 - | 4 | 2 | 0.06 |
| antibiotic-susceptible type strain. | | | |
| Staphylococcusaureus ATCC 25923 - | 4 | 1 | 0.06 |
| antibiotic-susceptible type strain. | | | |
| Staphylococcusaureus ATCC 43300 - | >64 | >64 | >64 |
| methicillin-resistant type strain. | | | |
| Staphylococcus epidermidis - antibiotic susceptible | 4 | 2 | 0.12 |
| clinical isolate. | | | |
| Staphylococcus epidermidis - methicillin-resistant | 8 | 4 | 0.12 |
| clinical isolate. | | | |
| Staphylococcus haemolyticus - antibiotic susceptible | 4 | 2 | 0.06 |
| clinical isolate. | | | |
| Staphylococcus saprophyticus - antibiotic susceptible | 4 | 1 | 0.06 |
| clinical isolate. | | | |
| Enterococcus faecalis - ATCC 29212 | >64 | >64 | 8 |
| antibiotic-susceptible type strain. | | | |
| Enterococcus faecium - vancomycin-susceptible | >64 | >64 | 32 |
| clinical isolate. | | | |
| Streptococcus pneumoniae - ATCC 49619 | 2 | 0.5 | ≦0.03 |
| antibiotic-susceptible type strain. | | | |
| Streptococcus pneumoniae - penicillin-susceptible | 1 | 0.5 | ≦0.03 |
| clinical isolate. | | | |
| Streptococcus pyogenes - antibiotic-susceptible clinical | 2 | 1 | ≦0.03 |
| isolate. | | | |
| Streptococcus pyogenes - Macrolide (MLS) resistant | 4 | 2 | ≦0.03 |
| clinical isolate | | | |
| Corynebacterium jeikeium - antibiotic-susceptible | >64 | >64 | >64 |
| clinical isolate. | | | |
| Listeria monocytogenes - antibiotic-susceptible clinical | 32 | 16 | 1 |
| isolate. | | | |
| Streptococcus agalactiae - antibiotic-susceptable | 2 | 1 | ≦0.03 |
| clinical isolate | | | |
| Group C Streptococcus - antibiotic-susceptible clinical | 2 | 1 | ≦0.03 |
| isolate | | | |
| Group G Streptococcus - antibiotic-susceptible clinical | 2 | 1 | ≦0.03 |
| isolate | | | |
| Streptococcus mitis - antibiotic-susceptible clinical | 0.5 | 0.25 | ≦0.03 |
| isolate | | | |
| Streptococcus constellatus - antibiotic-susceptible | 2 | 1 | ≦0.03 |
| clinical isolate | | | |
| Streptococcus oralis - antibiotic-susceptible clinical | 0.5 | 0.25 | ≦0.03 |
| isolate | | | |
| Helicobacter pylori - clinical isolate | ≦0.03 | ≦0.03 | ≦0.03 |
| Helicobacter pylori - clinical isolate | ≦0.03 | ≦0.03 | ≦0.03 |
| Helicobacter pylori - ATCC 43504 | ≦0.03 | ≦0.03 | ≦0.03 |

Prodrug 1 = clindamycin succinyl valine ester
Prodrug 2 = clindamycin glutaryl PABA ester

### Example 10

### Bioavailability of Clindamycin from its Succinyl Valine and Glutaryl Para-Amino Benzoic Acid Ester Prodrugs in the Rat

In order to confirm successful absorption and subsequent hydrolysis of these clindoamycin prodrugs, a comparative bioavailability study was undertaken in the rat.

Methods—Three groups of five male Sprague Dawley rats were orally dosed with either clindamycin itself or its succinyl valine or glutaryl para-amino benzoic acid ester prodrugs. Serial tail vein blood samples were subsequently collected via an indwelling cannuala into EDTA tubes containing a stabilising cocktail (including 6 mg/mL NaF) to prevent post collection enzymic hydrolysis of the prodrug. Separated plasma samples were subjected to bioanalysis for both the prodrugs and clindamycin itself using a qualified LC/MS-MS bioanalytical method. Subsequent pharmacokinetic analysis was then undertaken using Win NonLin to generate the relevant PK parameters including AUC and T_{1/2} & T_{50%}Cₘₐₓ the period for which plasma drug conentrations remained at or above 50% of the Cₘₐₓ.

Results—These are shown in Tables 9, 10 &11 inclusive

TABLE 9
Pharmacokinetics of clindamycin after oral dosing of 5
mg clindamycin free base equivalents/kg to male rats

| Pharmacokinetic | | | | | | | |
|---|---|---|---|---|---|---|---|
| parameter | 1 | 2 | 3 | 4 | 5 | Mean | sd |
| Cₘₐₓ (ng/mL) | 251 | 342 | 248 | 246 | 191 | 256 | 54 |
| Tₘₐₓ (h) | 0.25 | 0.25 | 0.5 | 0.25 | 0.25 | 0.30 | 0.11 |
| AUCt (ng · h/mL) | 445 | 638 | 622 | 382 | 274 | 472 | 157 |
| t½ (h) | 1.8 | 1.9 | 1.7 | 3.5 | 2.9 | 2.3 | 0.8 |
| T_{>50% Cmax} (h) | 1.0 | 1.1 | 1.8 | 0.8 | 0.8 | 1.1 | 0.4 |

TABLE 10
Pharmacokinetics of clindamycin after oral dosing of 5 mg clindamycin
equivalents of the succinyl valine ester prodrug/kg to male rats

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 6 | 11 | 6 | 4 | 10 | 7 | 3 |
| Tₘₐₓ (h) | 4.0 | 4.0 | 2.0 | 2.0 | 4.0 | 3.2 | 1.1 |
| AUCt (ng · h/mL) | 35 | 50 | 33 | 28 | 45 | 38 | 9 |
| t½ (h) | 3.7 | 2.1 | 2.8 | 4.2 | 2.4 | 3.0 | 0.9 |
| T_{>50% Cmax} (h) | 4.3 | 3.5 | 5.3 | 6.5 | 3.1 | 4.6 | 1.4 |
| Fᵣₑₗ (%) | | | | | | 8% | |

TABLE 11
Pharmacokinetics of clindamycin after oral dosing
of 5 mg clindamycin equivalents of the glutaryl
PABA ethyl ester prodrug/kg to male rats

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 57 | 72 | 51 | 39 | 53 | 54 | 12 |
| Tₘₐₓ (h) | 2.0 | 2.0 | 2.0 | 2.0 | 2.0 | 2.0 | 0.0 |
| AUCt (ng · h/mL) | 306 | 302 | 213 | 175 | 212 | 242 | 59 |
| t½ (h) | 2.7 | 2.1 | 2.1 | 1.9 | 1.9 | 2.1 | 0.3 |
| T_{>50% Cmax} (h) | 5.0 | 3.3 | 3.8 | 4.4 | 3.1 | 3.9 | 0.8 |
| Fᵣₑₗ (%) | | | | | | 51% | |

Following administration of clindamycin itself, peak plasma levels were seen very rapidly—within 15 mins of dosing. Subsequent elimination also took place rapidly with a half-life of −2 h. By contrast clindamycin plasma levels after either prodrug did not peak until around >2 h post dosing and, although significantly lower than after the parent drug, persisted for several hours. Thus the mean T_{50%}Cₘₐₓ (the period for which plasma clindamycin levels stayed at or above 50% of the maximum concentration) after the succinyl valine ester was 4.6±1.4 h compared with 1.1±0.4 h after giving clindamycin itself. Similarly this value, after the glutaryl PABA ester, was 3.9±0.8 h some three-fold longer than that after administration of the parent drug. The relative bioavailability of the drug after the succinyl valine prodrugs was 8% of that seen after giving the drug itself but that after the glutaryl PABA ester was 51%.

These result confirm the potential of these prodrugs to deliver the active drug to the systemic circulation but importantly also confer the advantage of prolonged plasma drug concentrations. If this translates to man it should mean less frequent dosing and increased patient compliance.

### Example 11

### Synthesis of propafenone-[succinyl-(S)-valine]ester hydrochloride

Nausea and vomiting associated with the antiarrhythmic propafenone are experienced in >10% of patients treated with the drug for supraventricular arrhythmias. This effect is believed to be a consequence of reduction in stomach slow wave activity and resultant gastric stasis and can be overcome by the use of transiently inactivated prodrugs described below.

Propafenone-[succinyl-(S)-valine]ester hydrochloride was prepared in four steps (Schemes 9 and10). Initially, propafenone hydrochloride was treated with di-tert-butyl dicarbonate to afford N-Boc-propafenone.

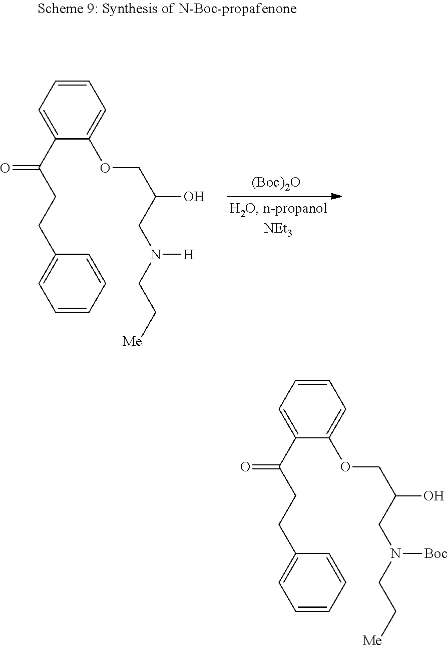

Subsequently (S)-valine tert-butyl ester hydrochloride was treated with triethylamine and succinic anhydride to give succinyl-(S)-valine tert-butyl ester which was coupled to N-Boc-propafenone via a N,N′-dicyclohexylcarbodiimide (DCC) mediated reaction to give N-Boc-propafenone-[succinyl-(S)-valine tert-butyl ester]ester.

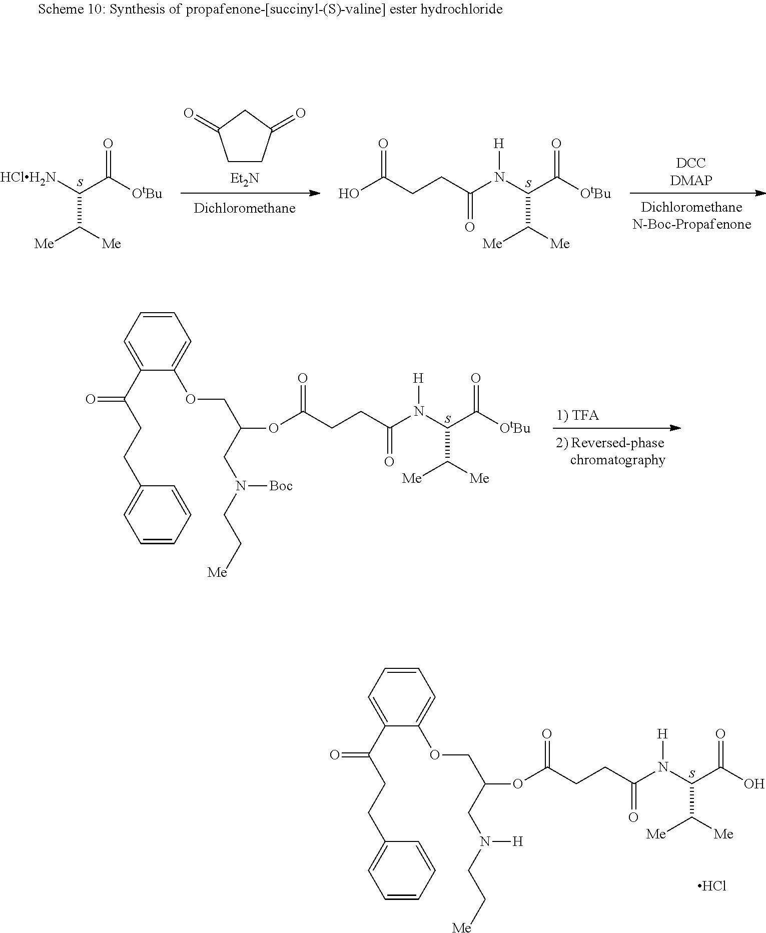

After purification, the tert-butyl ester and Boc group were cleaved using trifluoroacetic acid. Reversed-phase chromatography (with aqueous HCl in the mobile phase) afforded the desired propafenone-[succinyl-(S)-valine]ester hydrochloride as a colourless glassy-solid.

Detail

To a stirred solution of (S)-valine tert-butyl ester hydrochloride (5.00 g, 23.8 mmol) in anhydrous dichloromethane (125 mL) was added triethylamine (5.31 g, 7.31 mL, 52.5 mmol) and succinic anhydride (2.63 g, 26.3 mmol) and the mixture was stirred at room temperature overnight. The solution was diluted with dichloromethane (100 mL), washed with 5% citric acid (2×100 mL) and brine (100 mL), dried (MgSO₄) and concentrated to give succinyl-(S)-valine tert-butyl ester (6.4 g, 98%) as a white solid.

To a stirred solution of N-Boc-propafenone (0.80 g, 1.81 mmol) in anhydrous dichloromethane (20 mL) was added succinyl-(S)-valine tert-butyl ester (0.64 g, 2.36 mmol), 4-dimethylaminopyridine (11 mg, 0.09 mmol) and N,N-dicyclohexylcarbodiimide (0.52 g, 2.54 mmol), and the mixture was stirred at room temperature overnight. The resulting suspension was filtered through Celite and the filtrate was concentrated to afford a yellow gummy semi-solid (1.76 g) which was purified using a Biotage Isolera automated chromatography system under reversed-phase conditions (C₁₈ column, gradient of 0→100% MeCN in 0.02% aqueous HCl) with detection at 300 nm to afford, after freeze-drying, N-Boc-propafenone-[succinyl-(S)-valine tert-butyl ester]ester (1.06 g, 84%) as a colourless glassy solid.

N-Boc-propafenone-[succinyl-(S)-valine tert-butyl ester]ester (1.06 g, 1.52 mmol) in trifluoroacetic acid (15 mL) was stirred at room temperature for 1 h. The mixture was evaporated to dryness and residual trifluoroacetic acid was removed azeotropically with chloroform (5×35 mL) to afford a clear gummy semi-solid (1.26 g). Part of this material (0.63 g) was purified using a Biotage Isolera automated chromatography system under reversed-phase conditions (C₁₈ column, gradient of 0→100% MeCN in 0.02% aqueous HCl) with detection at 302 nm to afford, after freeze-drying, propafenone-[succinyl-(S)-valine]ester hydrochloride (0.28 g, 68%) as a colourless glassy solid.

### Example 12

### Propafenone[succinyl-(S)-valine]ENOL ester trifluoroacetate

The synthesis of propafenone[succinyl-(S)-valine]enol ester trifluoroacetate was accomplished in five distinct reaction steps as shown in scheme 11.

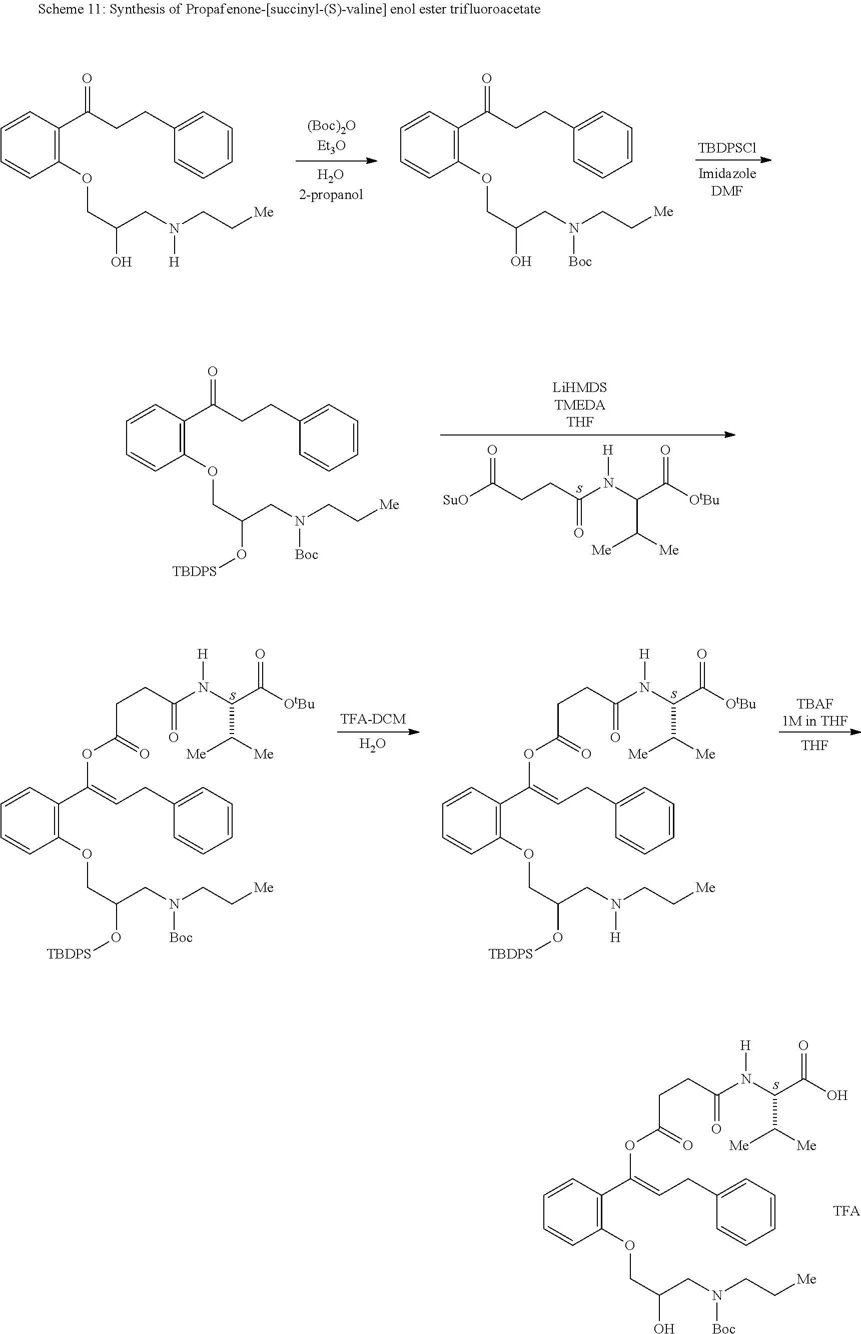

Propafenone hydrochloride was reacted with di-tert-butyl di-carbonate to give N-Boc-propafenone in excellent yield. The propafenone hydroxyl group was protected as its silyl ether using tert-butyldiphenylchlorosilane to give the fully protected propafenone. Formation of the enol ester derivative was achieved using lithium hexamethyldisilazide (LiHMDS) in the presence of N,N,N′,N′-tetramethyl-ethane-1,2-diamine (TMEDA) followed by addition of[succinyl-(S)-valine tert-butyl ester]N-hydroxysuccinimide ester. Deprotection of both the Boc and tert-butyl groups was carried out using a mixture of trifluoroacetic acid, dichloromethane and water. Finally, treatment of the partially deprotected propafenone enol ester with tetrabutylammonium fluoride in tetrahydrofuran afforded the required propafenone[succinyl-(S)-valine]enol ester trifluoroacetate.

Detail

To a suspension of propafenone hydrochloride (4.00 g, 10.6 mmol) in a mixture of water (100 mL) and 2-propanol (50 mL) was added triethylamine (5 mL) and the suspension was heated at 50° C. until a solution was obtained. The reaction was cooled to room temperature, di-tert-butyl di-carbonate (2.77 g, 12.7 mmol) was added and the mixture was stirred for 2.5 h. 2-Propanol was removed under vacuum and to the resulting suspension was added 25% aqueous orthophosphoric acid (50 mL). The aqueous layer was extracted with ethyl acetate (3×150 mL) and the organics were combined, dried (MgSO₄) and concentrated to give an pale-yellow oil. Filtration through a pad of silica (eluent dichloromethane:methanol, 94:6 v/v) and concentration of the resulting solution afforded N-Boc-propafenone (4.40 g, 94%), as a cream coloured solid.

To a solution of N-Boc-propafenone (2.00 g, 4.52 mmol) in DMF (8 mL) was added tert-butyldiphenylchlorosilane (1.62 g, 1.51 mL, 5.88 mmol) followed by imidazole (0.80 g, 11.8 mmol) and the mixture was stirred overnight at room temperature. The resulting solution was quenched with water (50 mL) and the mixture was extracted with diethyl ether (3×100 mL). The organics were combined and washed with water (5×100 mL), saturated brine (100 mL), dried (MgSO₄) and concentrated to give a viscous oil. This residual oil was purified by medium-pressure chromatography on silica eluting with a gradient of 10→15% ethyl acetate in petrol to give N-Boc-O-TBDPS-propafenone (3.10 g, quantitative) as a viscous oil.

R_{f} 0.78 (20% ethyl acetate-80% petrol).

To a 1M solution of lithium hexamethyldisilazide (5.29 mL, 5.29 mmol) in tetrahydrofuran (25 ml) cooled in an ice-bath was added N,N,N′,N′-tetramethyl-ethane-1,2-diamine (TMEDA) (0.62 g, 0.80 mL, 5.29 mmol) and the mixture was stirred for 10 min. The resulting mixture was cooled in a dry ice/acetone bath to −78° C. and a solution of N-Boc-O-TBDPS-propafenone (3.00 g, 4.41 mmol) in tetrahydrofuran (25 mL) was added dropwise over 20 min and the mixture was stirred for 1 h. To the resulting yellow solution was added [succinyl-(S)-valine tert-butyl ester]N-hydroxysuccinimide ester (2.44 g, 6.62 mmol) in one portion and the mixture was stirred overnight whilst the temperature was allowed to warm to room temperature. The suspension was filtered through Celite and the filtrate concentrated to give a yellow oil. This residual oil was purified by medium-pressure chromatography on silica eluting with a gradient of 10→20% ethyl acetate in petrol to give N-Boc-O-TBDPS-propafenone[succinyl-(S)-valine tert-butyl ester]enol ester (780 mg, 19%), as a white solid.

R_{f} 0.50 (20% ethyl acetate-80% petrol).

To a solution of N-Boc-O-TBDPS-propafenone[succinyl-(S)-valine tert-butyl ester]enol ester (100 mg, 0.11 mmol) in dichloromethane (6 mL) was added trifluoroacetic acid (5.4 mL) followed by water (0.6 mL) and the mixture was stirred overnight at room temperature. The resulting solution was concentrated to give O-TBDPS-propafenone[succinyl-(S)-valine]enol ester trifluoroacetate (50 mg, 71%), as a foamed oil.

To a solution of (O-TBDPS-propafenone[succinyl-(S)-valine]enol ester trifluoroacetate (50 mg, 0.06 mmol) in tetrahydrofuran (2 mL) was added 1M tetrabutylammonium fluoride in tetrahydrofuran (0.11 mL, 0.11 mmol) and the mixture was stirred for 2 h. The solution was concentrated and the residue was purified using a Biotage Isolera automated chromatography system under reversed-phase conditions (C₁₈ column, gradient of 0→100% MeCN in 0.1% aqueous TFA) with detection at 254 nm to afford, after freeze-drying, propafenone[succinyl-(S)-valine]enol ester trifluoroacetate (60 mg) as a white solid.

### Example 11

### Synthesis of propafenone-(glutaryl-PABA) ester hydrochloride

Propafenone-(glutaryl-PABA) ester hydrochloride was prepared from PABA tert-butyl ester and N-Boc-propafenone in four steps (Scheme 12).

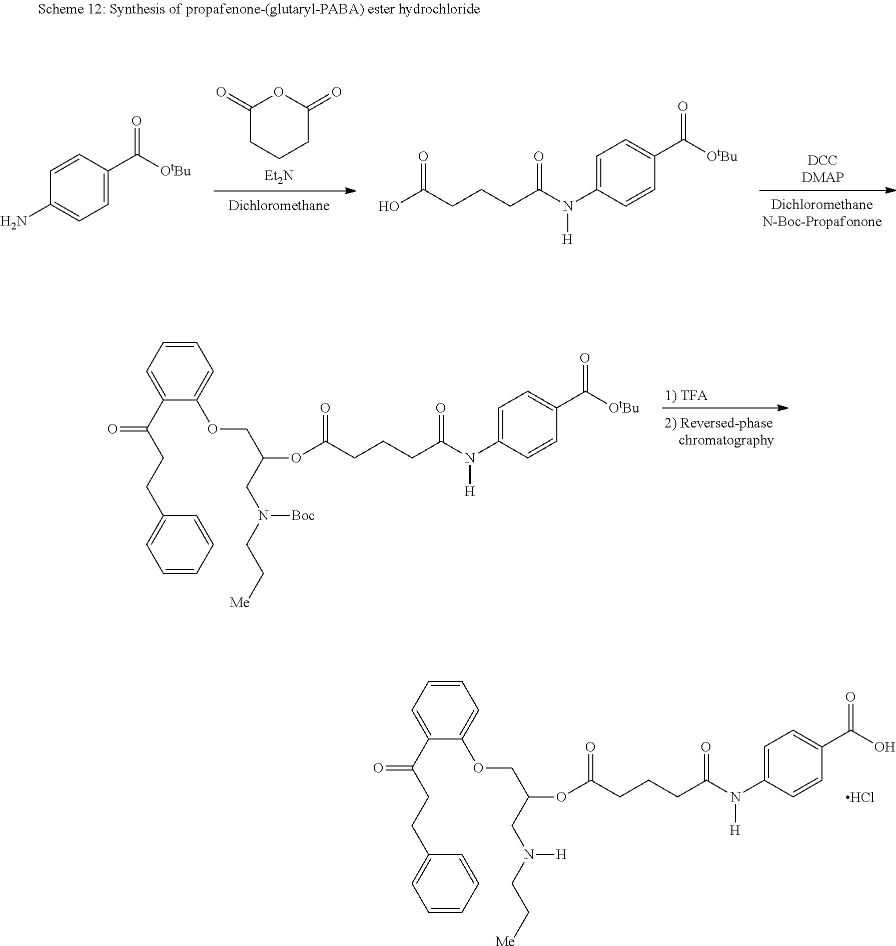

PABA tert-butyl ester was treated with triethylamine and glutaric anhydride to give glutaryl-PABA tert-butyl ester which was treated with N,N-dicyclohexylcarbodiimide and N-Boc-propafenone to give N-Boc-propafenone-(glutaryl-PABA tert-butyl ester) ester. After purification, the tert-butyl ester and Boc group were cleaved using trifluoroacetic acid. Reversed-phase chromatography (with aqueous HCl in the mobile phase) afforded the desired propafenone-(glutaryl-PABA) ester hydrochloride as a white solid.

Detail

To a stirred solution of PABA tert-butyl ester (2.50 g, 12.9 mmol) in ethyl acetate (125 mL) was added triethylamine (1.70 g, 2.35 mL, 16.8 mmol) and glutaric anhydride (2.21 g, 19.4 mmol), and the mixture was stirred at room temperature overnight. The solution was washed with 5% citric acid (3×75 mL), water (75 mL), and brine (100 mL), then dried (MgSO₄) and concentrated to give glutaryl-PABA tert-butyl ester (3.84 g, 97%) as a white solid.

To a stirred solution of N-Boc-propafenone (0.80 g, 1.81 mmol) in anhydrous dichloromethane (20 mL) was added glutaryl-PABA tert-butyl ester (0.72 g, 2.36 mmol), 4-dimethylaminopyridine (11 mg, 0.09 mmol) and N,N′-dicyclohexylcarbodiimide (0.52 g, 2.54 mmol), and the mixture was stirred at room temperature overnight. The resulting suspension was filtered through Celite and the filtrate concentrated to afford a yellow gummy semi-solid (1.67 g) which was purified using a Biotage Isolera automated chromatography system under reversed-phase conditions (C₁₈ column, gradient of 0→100% MeCN in 0.02% aqueous HCl) with detection at 260 nm to afford, after freeze-drying, N-Boc-propafenone-(glutaryl-PABA tert-butyl ester) ester (1.08 g, 90%) as a yellow gummy semi-solid.

N-Boc-Propafenone-(glutaryl-PABA tert-butyl ester) ester (1.08 g, 1.48 mmol) in trifluoroacetic acid (15 mL) was stirred at room temperature for 1 h. The mixture was evaporated to dryness and residual trifluoroacetic acid was removed azeotropically with chloroform (5×35 mL) to afford a pale yellow gummy semi-solid (1.21 g). Part of this material (0.61 g) was taken and purified using a Biotage Isolera automated chromatography system under reversed-phase conditions (C₁₈ column, gradient of 0→100% MeCN in 0.02% aqueous HCl) with detection at 269 nm to afford, after freeze-drying, propafenone-(glutaryl-PABA) ester hydrochloride (0.26 g, 62%) as a white solid.

### Example 12

### Assessment of Chemical/Biological Stability of Propafenone Prodrugs

To minimize the direct inhibitory action of this drug on gastric emptying/gut motility—and resultant nausea—the prodrug should not undergo premature chemical or enzymic cleavage back to the parent drug while within the gut lumen. To confirm this, the prodrugs were incubated with USP SGF (simulated gastric fluid), FeSSIF (fed state simulated intestinal fluid) and FaSSIF (fasted state simulated intestinal fluid) and remaining prodrug, and any generated active drug, assayed by use of a qualified LC-MS/MS analytical method.

Method

Prodrug (20 μg/mL) in the FaSSIF cocktail comprising sodium taurocholate 3 mM, lecithin 0.75 mM, NaOH 8.7 mM, NaH₂PO₄. H₂O 28.7 mM, NaCl 106 mM, pancreatin 1% (overall pH 6.5) was incubated for 2 h at 37° C. After acidifying the incubation to stop any reaction the amount of prodrug remaining and drug generated were determined using a qualified LC-MS/MS method.

The corresponding FeSSIF cocktail which was used in comparable manner comprised sodium taurocholate 15 mM, lecithin 3.75 mM, NaOH 101 mM, acetic acid 144 mM, and NaCl 202 mM (overall pH 5.0). Pancreatin was again added at 1%.

The USP simulated gastric juice comprised NaCl 34 mM, HCl ˜70 nM, pepsin 0.32% (overall pH1.1) and was incubated with prodrug (20 μg/mL) for 1 h at 37° C. Remaining prodrug and any generated active drug were assayed using a qualified LC-MS/MS method.

Results

The results shown in Table 12 indicate that both propafenone prodrugs were relatively chemically and enzymically stable under the conditions likely to prevail the GI tract. As such there should minimal potential for direct interaction of the active drug on the gut.

TABLE 12
Stability of propafenone succinyl valine ester and propafenone
glutaryl PABA ester in simulated gastric and intestinal juice

| | | Simulated | Simulated |
|---|---|---|---|
| | Simulated | intestinal juice | intestinal juice |
| | gastric juice | (2 h, 37° C.) | (2 h, 37° C.) |
| | (1 h, 37° C.) | (FaSSIF) | (FeSSIF) |
| | % active drug | % active drug | % active drug |
| Compound | generated | generated | generated |
| Propafenone succinyl | 1% | 9% | 1% |
| valine ester | | | |
| Propafenone glutaryl | 1% | 16% | 1% |
| PABA ester | | | |

### Example 13

### Effects of Propafenone and Two Propafenone Amino Acid Prodrugs on Cloned Nav1.1 Channels Expressed in Mammalian Cells

In order to confirm the loss of intrinsic local anesthetic activity (and likely cause of the directly mediated emesis) of propafenone the activity of two prodrugs of the compound (the succinyl valine and glutaryl PABA conjugates) were assessed in comparison with the parent drug molecule for their effects on the sodium 1.1 channel.

hNav1.1 Test Procedures

Blockade of hNav1.1 channel was measured using a stimulus voltage pattern shown in the diagram below: Voltage potentials are indicated in Table 13. The pulse pattern was repeated twice: before and 5 minutes after TA addition and peak current amplitudes at three test pulses were measured (ITP1, TP11 and ITP12).

TABLE 13
Voltage-protocol parameters for hNav1.1 channel

| | | | Test | | | Test |
|---|---|---|---|---|---|---|
| | | Pre- | Pulse | | | Pulse |
| | Holding | Pulse | 1-10, | Test | Inter- | 1-14 |
| | Poten- | Poten- | 12-14 | Pulse 11 | pulse | Poten- |
| | tial | tial | Duration | Duration | Duration | tial |
| Channel | (mV) | (mV) | (ms) | (ms) | (ms) | (mV) |
| Nav1.1 | −80 | −120 | 20 | 500 | 80 | 0 |

### Data Analysis

Data acquisition and analyses was performed using the lonWorks Quattro™ system operation software (version 2.0.2; Molecular Devices Corporation, Union City, Calif.). Data was corrected for leak current.

The tonic block was calculated as:

% Block (Tonic)=(1−I_{TP1,TA}/I_{TP1,Control})×100%,

where I_{TP1,Control} and I_{TP1, TA} are the inward peak Na⁺ currents elicited by the TP1 in control and in the presence of a test article, respectively.

10 Hz Block—the frequency-dependent block at stimulation frequency 10 Hz was calculated as:

% Block (10 Hz)=(1−I_{TP11, TA}/I_{TP11, Control}))×100%,

where I_{TP11, Control} and I_{TP11, TA} are the inward peak Na⁺ currents elicited by the TP11 in control and in the presence of a test article, respectively.

We define the inactivation state block as decrease in test pulse (TP12) current amplitude due to the conditioning depolarizing pulse (TP11). The inactivation state block was calculated as:

% Block (inactivation state)=(1−(I_{TP12, TA}/I_{TP12, TA})×100%,

where I_{TP12, Control} and I_{TP12, TA} are the inward peak Na⁺ currents elicited by the TP12 in control and in the presence of a test article, respectively.

Concentration-response data for the blocks were fit to an equation of the following form:

% Block={1−1/[1+([Test]/IC₅₀)^{N}]}*100%,

where [Test] is the concentration of test article, IC₅₀ is the concentration of the test article producing half-maximal inhibition, N is the Hill coefficient, and % Block is the percentage of ion channel current inhibited at each concentration of the test article. Nonlinear least squares fits were solved with the Solver add-in for Excel 2000 (Microsoft, Redmond, Wash.).

Results The results are shown in Table 14 below:—

TABLE 14
Summary Effects of various propafenone prodrugs on hNav1.1 Channel

| | IC₅₀ (μM) | IC₅₀ (μM) | IC₅₀ (μM) |
|---|---|---|---|
| Compound | 10 Hz block | Tonic block | Inactivated state |
| Propafenone | 5.89 | 13.1 | 3.99 |
| Propafenone succinyl | 69.5 | 151 | 37.2 |
| valine ester | | | |
| Propafenone glutaryl | 96.3 | 464 | 32.2 |
| PABA ester | | | |

As can be seen in Table 13 the prodrugs had IC50 values for sodium channel blockade which were profoundly lower than the IC50 value for the parent drug suggesting that they should have much reduced effects on eliciting the gastric stasis and consequent emesis seen with the parent drug.

### Example 14

### Synthesis of miglustat 2-succinyl valine ester

The ceramide synthase inhibitory activity of miglustat is not only responsible for its therapeutic activity but also the unwanted GI adverse events due to direct interaction with gut disaccharidases. Transient inactivation by substitution at the 2-position (known to be critical for the drug's activity [Boucheron C et al (2006) Tetrahedron: Asymmetry 16 1747-1756]), while the drug is still present in the gut lumen, should minimise this effect.

This synthesis was effected using the route outlined below in scheme 13:—

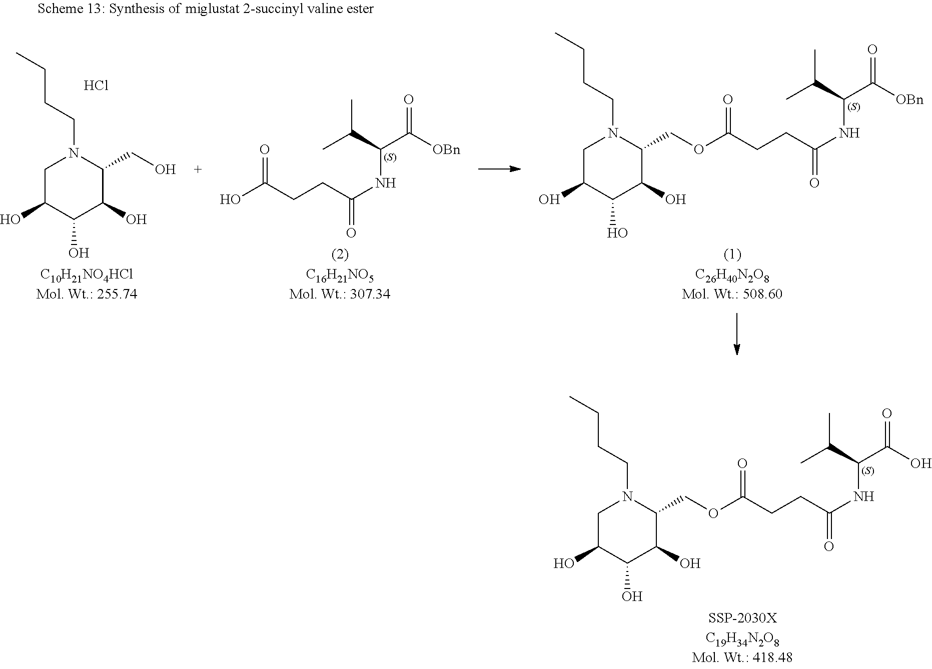

### Detail—Step 1

### Benzyl(2S)-2-(4-{[(2R,3R,4R,5S)-1-butyl-3,4,5-trihydroxy-piperidin-2-yl]methoxy}-4-oxobutanamido)-3-methylbutanoate (1)

Miglustat HCl (70 mg, 0.27 mmol) and triethylamine (71 mg, 0.70 mmol) were stirred in acetonitrile (4 mL). The suspension was gently warmed to give a solution. A mixture of benzylvaline succinic acid (2) (80 mg, 0.27 mmol), DMAP (10 mg, 0.08 mmol) and EDCl.HCl (67 mg, 0.35 mmol) in acetonitrile (2 mL) was charged and the resulting mixture stirred at 15-25° C. for 17 hours. The mixture was partitioned between ethyl acetate (40 mL), saturated brine (40 mL) and 2N sodium hydroxide solution (5 mL). The ethyl acetate phase was dried over magnesium sulphate and evaporated in vacuo at 40° C. to yield a clear yellow oil (133 mg) which was analysed by LCMS and shown to contain a component consistent with the desired product. The oil was dissolved in ethyl acetate (20 mL) and was extracted into 2M aqueous hydrochloric acid (10 mL) and then water (10 mL). The aqueous phases were combined and the pH was adjusted to 9 with saturated aqueous sodium hydrogen carbonate (20 mL). The aqueous phase was then extracted with ethyl acetate (2×35 mL), dried over magnesium sulphate and evaporated in vacuo at 40° C. to yield a clear oil (52.8 mg). The oil was purified by column chromatography on silica (40 g) eluting with 5% MeOH/DCM (75×25 mL) to yield benzyl (2S)-2-(4-{[(2R,3R,4R,5S)-1-butyl-3,4,5-tri hydroxy-piperidin-2-yl]methoxy}-4-oxobutanamido)-3-methyl but anoate (1), 12.2 mg) as a clear oil. Further elution with 10% MeOH/DCM (7×25 mL) yielded additional (1) (5.1 mg) as a clear oil.

¹H NMR (300 MHz, CDCl₃): δ 7.3 ppm (brs, 5H), 6.5 (brd, 1H), 5.1 (dd (app q), 2H), 4.5 (dd, 1H), 4.4 (d, 1H), 4.25 (d, 1H), 3.60 (m, 1H), 3.35 (t, 1H), 3.25 (t, 1H), 3.0 (m, 1H), 2.50-2.40 (m, 5H), 2.3-2.0 (m, 3H), 1.5-1.1 (m, 5H), 0.9-0.7 (m, 9H).

Mass spec: ES+: m/z 509.73 (M+1); ES−: m/z 543.06 (M+HCl),

TLC analysis: eluant 10% MeOH/DCM, R_{f} 0.26, visualised by UV and KMnO₄).

### Detail—Step 2

### (2S)-2-(4-{[(2R,3R,4R,5S)-1-butyl-3,4,5-trihydroxypiperidin-2-yl]methoxy}-4-oxobutanamido)-3-methylbutanoic acid, (Miglustat 2-succinyl valine)

A solution of benzyl (2S)-2-(4-{[(2R,3R,4R,5S)-1-butyl-3,4,5-trihydroxy-piperidin-2-yl]methoxy}-4-oxobutanamido)-3-methylbutanoate (1) (KK345-057-06/07, 15 mg) in ethanol (2 mL) was stirred over 10% Pd/C (50% wet) (27 mg) under an atmosphere of hydrogen for 42 hours after which time TLC analysis (eluant 10% MeOH/DCM, visualised by UV and KMnO₄) indicated no remaining 1. The suspension was filtered through celite and the filtrate evaporated in vacuo at 40° C. to yield a clear oil. The oil was triturated in dichloromethane (2 mL) and then evaporated in vacuo at 40° C. to yield (2S)-2-(4-{[(2R,3R,4R,5S)-1-butyl-3,4,5-trihydroxypiperidin-2-yl]methoxy}-4-oxobutanamido)-3-methylbuta noic acid, 11.1 mg) as an hygroscopic white solid.

¹H NMR (300 MHz, d₆-DMSO): δ 12.5 ppm (brs, 1H), 8.1 (brd, 1H), 5.0-4.7 (brm, 2H), 4.45 (brd, 1H), 4.2 (dd, 1H), 4.10 (m, 1H), 3.50-2.0 (br signals, 9H), 1.5-1.2 (m, 5H), 1.0-0.9 (2×d, 9H).

LCMS (10-0924): >98.0% [ES−: m/z 417.2 (M−1)]

### Example 15

### Synthesis of Donepezil Prodrugs

In order to avoid the direct effects of donepezil on gut epithelial acetyl choline esterase and the resultant emesis/diarrhoea the drug was transiently inactivated by prodrugging in the manner described below.

Donepezil Olutaryl Enol Ester Hydrochloride & Glutaryl Valine Ester (as its Ethyl Ester)

Synthesis of the donepezil glutaryl enol ester and also its glutaryl valine ester (as its ethyl ester), was undertaken as depicted in scheme 14:

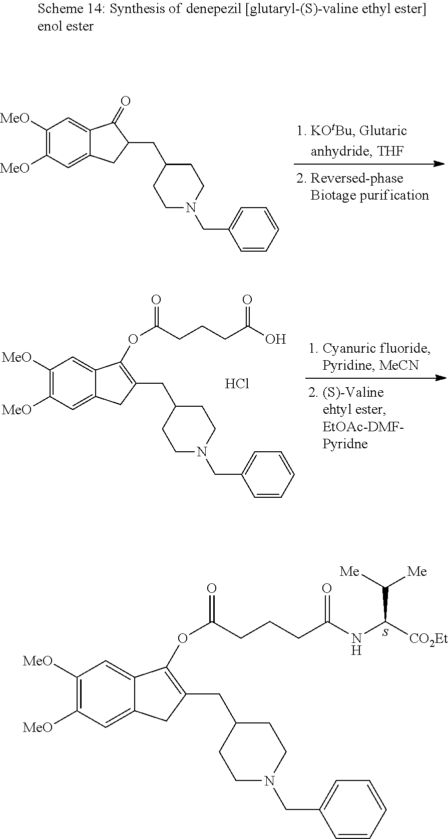

Donepezil was treated with potassium tert-butoxide and the resulting enolate was then quenched with glutaric anhydride. Purification was accomplished by reversed-phase chromatography. Treatment with cyanuric fluoride in the presence of pyridine gave the acid fluoride which was reacted with (S)-valine ethyl ester. The crude product was purified by normal phase chromatography to give the desired donepezil (glutaryl-(S)-valine ethyl ester) enol ester in ˜90% purity by ¹H NMR and HPLC.

Detail

Synthesis of Donepezil glutaryl enol ester hydrochloride

To a stirred solution of donepezil (1.5 g, 3.96 mmol) in THF (75 mL) at room temperature was added potassium tert-butoxide (0.49 g, 4.35 mmol) and the mixture was stirred for 30 min. To the resulting yellow solution was added glutaric anhydride (0.54 g, 4.75 mmol) and the mixture was stirred overnight. The resulting mixture was concentrated and the gummy residue purified using a Biotage Isolera automated chromatography system under reversed-phase conditions (C₁₈ column, gradient of 0→100% MeCN in 0.02% hydrochloric acid) with detection at 254 nm to afford, after freeze-drying, donepezil glutarate enol ester hydrochloride (0.62 g, 30%), as a white solid.

¹H NMR (DMSO-d₆): 10.56 (br s, 1H, CO₂H), 10.29 (s, 1H, NH⁺), 7.58 (m, 2H, 2×ArH), 7.44 (m, 3H, 3×ArH), 7.08 (s, 1H, ArH), 6.67 (s, 1H, ArH), 4.23 (m, 2H, NCH₂), 3.75 (s, 3H, OMe), 3.72 (s, 3 H, OMe), 3.28 (m, 4H, 2×NCH₂), 3.06 (m, 1H, CH), 2.83 (m, 2H, CH₂), 2.71 (t, J=7.2 Hz, 2H, CH₂), 2.37 (t, J=7.2 Hz, 2H, CH₂), 2.22 (d, J=6.9 Hz, 2H, CH₂), 1.89 (t, J=7.2 Hz, 2H, CH₂), 1.74 (m, 2H, CH₂), 1.49 (m, 2H, CH₂).

Synthesis of Donepezil (glutaryl acyl fluoride) enol ester

To a stirred solution of donepezil glutarate enol ester hydrochloride (0.62 g, 1.17 mmol) in acetonitrile (50 mL) was added pyridine (93 mg, 95 μL, 1.17 mmol) followed by cyanuric fluoride (63 mg, 40 □L, 0.47 mmol). The mixture was stirred for 30 min, diethyl ether (100 mL) was added and the solution washed with brine (100 mL). The organic layer was separated, dried (MgSO₄) and concentrated to give the acid fluoride (0.58 g), as a yellow oil.

Synthesis of Donepezil glutaryl-(S)-valine (OEt) enol ester

To a stirred solution of the acid fluoride (prepared as detailed above) (0.58 g, 1.17 mmol) in ethyl acetate (50 mL) and N,N-dimethylformamide (10 mL) was added pyridine (186 mg, 0.19 mL, 2.34 mmol) and (S)-valine ethyl ester (0.32 g, 1.76 mmol) and the mixture was stirred overnight. The resulting mixture was washed with water (5×50 mL), dried (MgSO₄) and concentrated to give a yellow solid which was purified using a Biotage Isolera automated chromatography system under normal phase conditions (silica column, gradient of 0→20% MeOH in dichloromethane) with detection at 254 nm to afford, after drying in vacuo at 40° C. for 3 h, donepezil[glutaryl-(S)-valine ethyl ester]enol ester (180 mg, 23%), as a yellow gum in ˜90% purity by ¹H NMR and HPLC.

Donepezil Glutaryl Paba Enol Ester (as its Ethyl Ester)

Synthesis of the donepezil glutaryl PABA enol ester (as its ethyl ester), was undertaken as depicted in scheme 15:

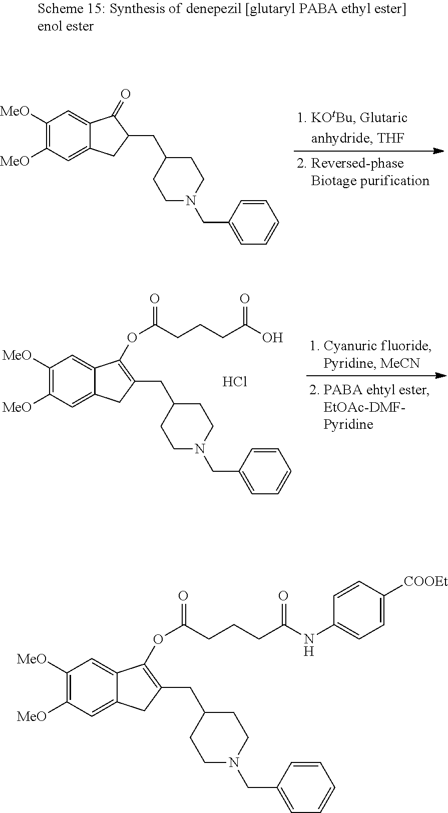

Donepezil was treated with potassium tert-butoxide and the resulting enolate was then quenched with glutaric anhydride. Purification was accomplished by reversed-phase chromatography. Treatment with cyanuric fluoride in the presence of pyridine gave the acid fluoride which was reacted with para-aminobenzoic acid ethyl ester. The crude product was purified by normal phase chromatography to give the desired donepezil (glutaryl-PABA ethyl ester) enol ester as a yellow gum.

Detail

To a stirred solution of donepezil (1.5 g, 3.96 mmol) in THF (75 mL) at room temperature was added potassium tert-butoxide (0.49 g, 4.35 mmol) and the mixture was stirred for 30 min. To the resulting yellow solution was added glutaric anhydride (0.54 g, 4.75 mmol) and the mixture was stirred overnight. The resulting mixture was concentrated and the gummy residue purified using a Biotage Isolera automated chromatography system under reversed-phase conditions (C₁₈ column, gradient of 0→100% MeCN in 0.02% hydrochloric acid) with detection at 254 nm to afford, after freeze-drying, donepezil glutarate enol ester hydrochloride (0.62 g, 30%), as a white solid.

To a stirred solution of donepezil glutarate enol ester hydrochloride (0.62 g, 1.17 mmol) in acetonitrile (50 mL) was added pyridine (93 mg, 95 ∞L, 1.17 mmol) followed by cyanuric fluoride (63 mg, 40 □L, 0.47 mmol). The mixture was stirred for 30 min, diethyl ether (100 mL) was added and the solution washed with brine (100 mL). The organic layer was separated, dried (MgSO₄) and concentrated to give the acid fluoride (0.58 g), as a yellow oil.

To a stirred solution of the acid fluoride (0.58 g, 1.17 mmol) in ethyl acetate (50 mL) and N,N-dimethylformamide (10 mL) was added pyridine (186 mg, 0.19 mL, 2.34 mmol) and 4-aminobenzoic acid ethyl ester (0.35 g, 1.76 mmol) and the mixture was stirred overnight. The resulting mixture was washed with water (5×50 mL), dried (MgSO₄) and concentrated to give a yellow solid which was purified using a Biotage Isolera automated chromatography system under normal phase conditions (silica column, gradient of 0→20% MeOH in dichloromethane) with detection at 254 nm to afford a yellow gum that was dryied in vacuo at 40° C. for 3 h to give donepezil[glutaryl-PABA ethyl ester]enol ester (51 mg, 7%), as a yellow gum in >90% purity by ¹H NMR and HPLC.

¹H NMR (DMSO-d₆): 10.34 (s, 1H, NH), 7.93 (d, J=8.7 Hz, 2H, 2×ArH), 7.75 (d, J=8.7 Hz, 2 H, 2×ArH), 7.27 (m, 5H, 5×ArH), 7.07 (s, 1H, ArH), 6.69 (s, 1H, ArH), 4.27 (q, J=7.2 Hz, 2H, OCH₂), 3.74 (s, 3H, OMe), 3.72 (s, 3H, OMe), 3.49 (m, 2H, CH₂), 3.25 (s, 2H, NCH₂), 2.89 (s, 2H, CH₂), 2.76 (m, 2H, NCH₂), 2.53 (m, 2H, NCH₂), 2.21 (d, J=6.0 Hz, 2H, CH₂), 2.00 (m, 3H, CH and CH₂), 1.56 (m, 3H, 1.5×CH₂), 1.30 (t, J=7.2 Hz, 3H, CH₃), 1.14 (m, 3H, 1.5×CH₂).

### Donepezil (Glutaryl-(S)-Valinamide) Enol Ester

Synthesis of the donepezil (glutaryl-(S)-valinamide) enol ester was undertaken as depicted in scheme 16:

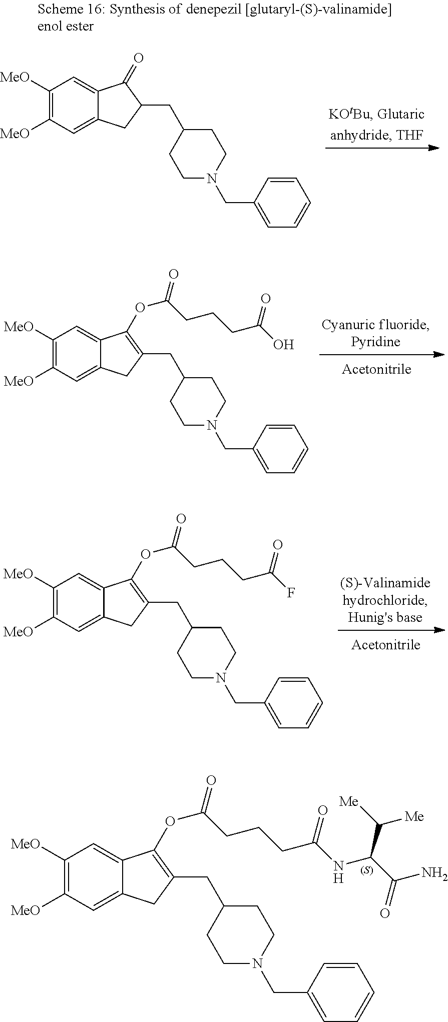

Donepezil was treated with potassium tert-butoxide in THF to give donepezil enolate which was quenched with glutaric anhydride. The free base was purified at this stage by normal phase chromatography. Conversion to the acid fluoride was achieved by addition of cyanuric fluoride to a solution of the free base in acetonitrile in the presence of pyridine. Reaction of the acid fluoride with (S)-valinamide hydrochloride in the presence of Hunig's base followed by normal phase chromatography gave donepezil [glutaryl-(S)-valinamide]enol ester as an orange glassy semi-solid.

Detail

To a stirred solution of donepezil glutaryl enol ester (prepared as detailed in 14.1; 0.81 g, 1.63 mmol) and pyridine (0.52 g, 0.53 ml, 6.52 mmol) in anhydrous acetonitrile (50 mL) under nitrogen was added cyanuric fluoride (220 mg, 140 μL, 1.63 mmol) and the mixture was then stirred for 1 h. Di-isopropylethylamine (421 mg, 0.57 mL, 3.26 mmol) and (S)-valinamide hydrochloride (499 mg, 3.26 mmol) were added and the mixture was stirred overnight. The resulting mixture was concentrated and the residue was purified using a Biotage Isolera automated chromatography system under normal phase conditions (silica column, gradient of 0→30% methanol in dichloromethane) with detection at 272 nm to afford donepezil[glutaryl-(S)-valinamide]enol ester (405 mg, 42%) as an orange glassy semi-solid. R_{f} 0.30 [dichloromethane-methanol, 4:1 v/v].

¹H NMR (DMSO-d₆): 7.84 (d, J=9.0 Hz, 1H, amide NH), 7.40 (br s, 2H, 2×ArH), 7.33 (br s, 4 H, 4×ArH), 7.08 (s, 1H, 0.5×valinamide NH₂), 7.03 (s, 1H, 0.5×valinamide NH₂), 6.68 (s, 1H, ArH), 4.13 (m, 1H, valinamide α-H), 3.74 (s, 3H, OCH₃), 3.72 (s, 3H, OCH₃), 3.34 (m, 5H, 2×NCH₂ and 0.5×CH₂), 3.26 (br s, 2H, NCH₂), 3.16 (d, J=5.1 Hz, 1H, 0.5×CH₂), 2.66 (t, J=7.5 Hz, 2H, CH₂), 2.32 (m, 2H, CH₂), 2.21 (m, 2H, CH₂), 1.92 (m, 4H, valinamide 13-H, CH and CH₂), 1.60 (br, 2H, CH₂), 1.23 (br, 2H, CH₂), 0.85 (t, J=6.6 Hz, 6H, 2×valinamide CH₃).

### Example 15

### Assessment of Chemical/Biological Stability of Donepezil Prodrugs

To avoid direct interaction between the active drug and the gut, the prodrug should not undergo premature chemical or enzymic cleavage back to the parent dug while within the gut lumen. To confirm this the prodrugs were incubated with USP SGF (simulated gastric fluid), FeSSIF (fed state simulated intestinal fluid) and FaSSIF (fasted state simulated intestinal fluid) and remaining prodrug and any generated active drug assayed by use of a qualified LC-MS/MS analytical method.

Method

Prodrug (20 μg/mL) in the FaSSIF cocktail comprising sodium taurocholate 3 mM, lecithin 0.75 mM, NaOH 8.7 mM, NaH₂PO₄. H₂O 28.7 mM, NaCl 106 mM, pancreatin 1% (overall pH 6.5) was incubated for 2 h at 37° C. After acidifying the incubation to stop any reaction the amount of prodrug remaining and drug generated were determined using a qualified LC-MS/MS method.

The corresponding FeSSIF cocktail, which was used in comparable manner, comprised sodium taurocholate 15 mM, lecithin 3.75 mM, NaOH 101 mM, acetic acid 144 mM, and NaCl 202 mM (overall pH 5.0). Pancreatin was again added at 1%.

The USP simulated gastric juice comprised NaCl 34 mM, HCl ˜70 nM, pepsin 0.32% (overall pH1.1) and was incubated with prodrug (20 μg/mL) for 1 h at 37° C. Remaining prodrug and any generated active drug were assayed using a qualified LC-MS/MS method.

Results

The results shown in Table 15 indicate that these donepezil prodrugs did not give rise to significant amounts of the active drug under the conditions likely to prevail the GI tract. As such there should minimal potential for direct interaction between active drug and the gut.

TABLE 15
Stability of various donepezil prodrugs
in simulated gastric and intestinal juice

| | | Simulated | Simulated |
|---|---|---|---|
| | | intestinal juice | intestinal juice |
| | Simulated gastric | (2 h, 37° C.) | (2 h, 37° C.) |
| | juice (1 h, 37° C.) | (FaSSIF) | (FeSSIF) |
| | % active drug | % active drug | % active drug |
| Compound | generated | generated | generated |
| Donepezil glutaryl | 2% | 11% | 3% |
| valine ethyl | | | |
| ester | | | |
| Donepezil glutaryl | 2% | 2% | 1% |
| PABA ethyl | | | |
| ester | | | |
| Donepezil glutaryl | <2.0% | <2.0% | <2.0% |
| enol ester | | | |
| Donepezil glutaryl | <2.0% | <2.0% | <2.0% |
| valinamide | | | |

### Example 16

### In Vitro Activity of Donepezil, Donepezil Glutaryl Enol Ester, Donepezil Glutaryl Valine(OEt) Enol Ester, Donepezil Glutaryl Valinamide Enol Ester and Glutaryl PABA(OEt) Enol Ester Prodrugs on Human Acetylcholine Esterase

Methods

Drug and prodrugs were assayed for their effects on human acetylcholineesterase activity in a standard in vitro assay at CEREP.

Results The results are shown in Table 16 below:—

TABLE 16
Summary effects of various donepezil prodrugs on hAChE Activity

| Compound | IC₅₀ (μM) |
|---|---|
| Donepezil | 0.033 |
| Donepezil glutaryl enol ester | 0.83 |
| Donepezil glutaryl-(S)-valine(OEt) enol ester | 0.32 |
| Donepezil glutaryl-(S)-valinamide enol ester | 4.0 |
| Donepezil glutaryl-PABA(OEt) enol ester | 0.30 |

As can be seen in Table 18 the prodrugs had IC50 values for hAChE activity ranging from 10-121-fold less potent than the IC50 value for the parent drug suggesting that they should have reduced effects on eliciting the emesis seen with the parent drug.

### Example 17

### Bioavailability of Donepezil from its Glutaryl Valine (as Ethyl Ester) and Glutaryl Para-Amino Benzoic Acid (as Ethyl Ester) Prodrugs in the Rat

In order to confirm successful absorption and subsequent hydrolysis of these donepezil prodrugs, a comparative bioavailability study was undertaken in the rat

Methods—Three groups of five male Sprague Dawley rats were orally dosed with either donepezil itself or its glutaryl valine (ethyl ester) or glutaryl para-amino benzoic acid (ethyl ester) ester prodrug. Serial tail vein blood samples were subsequently collected via an indwelling cannuala into EDTA tubes containing a stabilising cocktail (including 6 mg/mL NaF) to prevent post collection enzymic hydrolysis of the prodrug. Separated plasma samples were subjected to bioanalysis for both the prodrugs and donepezil itself using a qualified LC/MS-MS bioanalytical method. Subsequent pharmacokinetic analysis was then undertaken using Win NonLin to generate the relevant PK parameters including AUC and T_{1/2} & T_{50%}Cₘₐₓ, the period for which plasma drug concentrations remained at or above 50% of the Cₘₐₓ.

Results—These are shown in Tables 17, 18, & 19.

TABLE 17
Pharmacokinetics of donepezil after oral dosing of 1
mg donepezil free base equivalents/kg to male rats

| Pharmacokinetic | | | | | | | |
|---|---|---|---|---|---|---|---|
| parameter | 1 | 2 | 3 | 4 | 5 | Mean | sd |
| Cₘₐₓ (ng/mL) | 5.3 | 5.4 | 2.25 | 4.42 | 9.62 | 5.4 | 2.7 |
| Tₘₐₓ (h) | 1.0 | 0.5 | 0.5 | 0.5 | 0.5 | 0.6 | 0.22 |
| AUCt (ng · h/mL) | 27.4 | 18.9 | 19.4 | 22.2 | 28.1 | 23.2 | 4.4 |
| t½ (h) | ND | 3.7 | ND | ND | 2.6 | 3.1 | — |
| T_{>50% Cmax} (h) | 3.9 | 1.9 | 9.8 | 5.1 | 2.0 | 4.5 | 3.34 |

TABLE 18
Pharmacokinetics of donepezil after oral dosing
of 1 mg donepezil equivs of the glutaryl valine
ethyl ester prodrug equivalents/kg to male rats

| Pharmacokinetic | | | | | | | |
|---|---|---|---|---|---|---|---|
| parameter | 6 | 7 | 8 | 9 | 10 | Mean | sd |
| Cₘₐₓ (ng/mL) | 0.8 | 3.6 | 2.4 | 1.9 | 1.1 | 1.9 | 1.1 |
| Tₘₐₓ (h) | 0.5 | 0.5 | 0.5 | 0.5 | 8.0 | 2.0 | 3.4 |
| AUCt (ng · h/mL) | 3.9 | 14.9 | 4.1 | 4.8 | 8.4 | 7.2 | 4.6 |
| t½ (h) | ND | 4.7 | ND | ND | ND | 4.7 | — |
| T_{>50% Cmax} (h) | 6.3 | 2.9 | 1.3 | 1.5 | 8.0 | 4.0 | 3.0 |
| Fᵣₑₗ (%) | — | — | — | — | — | 31% | |

TABLE 19
Pharmacokinetics of donepezil after oral dosing
of 1 mg donepezil equivs of the glutaryl PABA ethyl
ester prodrug equivalents/kg to male rats

| Pharmacokinetic | | | | | | | |
|---|---|---|---|---|---|---|---|
| parameter | 11 | 12 | 13 | 14 | 15 | Mean | sd |
| Cₘₐₓ (ng/mL) | 2.2 | 1.0 | 1.0 | 0.75 | 0.5 | 1.12 | 0.6 |
| Tₘₐₓ (h) | 0.5 | 0.5 | 0.5 | 0.5 | 2.0 | 0.8 | 0.7 |
| AUCt (ng · h/mL) | 13.9 | 5.9 | 4.7 | 4.1 | 5.6 | 6.9 | 4.0 |
| t½ (h) | 4.5 | 6.4 | ND | ND | ND | 5.5 | — |
| T_{>50% Cmax} (h) | 6.5 | 6.4 | 6.1 | 6.9 | 8.5 | 6.9 | 0.9 |
| Fᵣₑₗ (%) | — | — | — | — | — | 30% | |

Following administration of donepezil itself, peak plasma levels were seen very rapidly—within 35 mins of dosing. Subsequent elimination also took place with a half-life of ˜3 h. By contrast donepezil plasma levels after the glutaryl PABA ethyl ester prodrug although significantly lower than after the parent drug, persisted for several hours. Thus the mean T50% Cmax (the period for which plasma donepezil levels stayed at or above 50% of the maximum concentration) after the glutaryl PABA prodrug prodrug was 6.9 h compared with 4.5 h after giving donepezil itself. The relative bioavailability after this prodrug was ˜30% of that seen after giving the drug itself. Prodrug plasma levels were undetectable. A similar profile was evident for the glutaryl valine ethyl ester prodrug although plasma persistence was not in evidence.

These result confirm the potential of these prodrugs to deliver the active drug to the systemic circulation but importantly may confer the advantage of prolonged plasma drug concentrations especially after the glutaryl PABA conjugate. If this translates to man it should mean less frequent dosing and increased patient compliance.

### Example 18

### Bioavailability of Donepezil from its Glutaryl Enol Ester and Glutaryl-(S)-Valinamide Enol Ester Prodrugs in the Male Beagle Dog and Cynomolgus Monkey

Dogs

In order to confirm successful absorption and subsequent hydrolysis of these donepezil prodrugs, a comparative bioavailability study was undertaken in the male beagle dog.

Methods

In a three-way cross over design five male beagle dogs were orally dosed with either donepezil itself or its glutaryl enol ester or glutaryl valinamide enol ester prodrug. Serial blood samples (0.5 mL) were subsequently collected from a suitable vein over 24 h, into tubes containing 1.75 mg/mL K₃EDTA, and 25 μL of 1M citrate buffer pH 3.0 with 20 mM 1,4-saccharolactone; acting as a pro-drug stabiliser and β-glucuronidase inhibitor, respectively to prevent post collection enzymic hydrolysis of the prodrug. Separated plasma samples were subjected to bioanalysis for both the prodrugs and donepezil itself using a qualified LC/MS-MS bioanalytical method. Subsequent pharmacokinetic analysis was then undertaken using Win NonLin to generate the relevant PK parameters including Cₘₐₓ, Tₘₐₓ and AUC and T_{1/2}.

Results—These are shown in Tables 20, 21, & 22.

TABLE 20
Pharmacokinetics of donepezil after oral dosing of 1
mg donepezil free base equivalents/kg to male dogs

| Pharmacokinetic | | | | | | | |
|---|---|---|---|---|---|---|---|
| parameter | 205 | 207 | 209 | 211 | 213 | Mean | sd |
| Cₘₐₓ (ng/mL) | 44 | 99 | 24 | 16 | 9 | 38 | 36 |
| Tₘₐₓ (h) | 1.0 | 0.5 | 1.0 | 1.0 | 1.0 | 1.0 | (0.5-1) |
| AUCt (ng · h/mL) | 208 | 501 | 69 | 45 | 23 | 169 | 199 |
| AUC (ng · h/mL) | 213 | 518 | 76 | 46 | 25 | 176 | 205 |
| t½ (h) | 4.5 | 5.0 | 3.5 | 2.3 | 1.8 | 3.4 | 1.4 |
| T_{>50% Cmax} (h) | 2.3 | 2.5 | 1.6 | 1.7 | 2.0 | 2.0 | 0.4 |

TABLE 21
Pharmacokinetics of donepezil after oral dosing of 1 mg donepezil
equivalents of the glutaryl enol ester prodrug/kg to male dogs

| Pharmacokinetic | | | | | | | |
|---|---|---|---|---|---|---|---|
| parameter | 205 | 207 | 209 | 211 | 213 | Mean | sd |
| Cₘₐₓ (ng/mL) | 33 | 19 | 22 | 22 | 5 | 20 | 10 |
| Tₘₐₓ (h) | 0.5 | 1.0 | 1.0 | 0.5 | 2.0 | 1.0 | (0.5-2) |
| AUCt (ng · h/mL) | 197 | 149 | 90 | 64 | 23 | 104 | 69 |
| AUC (ng · h/mL) | 203 | 153 | 97 | 66 | 23 | 109 | 71 |
| t½ (h) | 4.9 | 4.8 | 7.6 | 2.6 | 2.5 | 4.5 | 2.1 |
| T_{>50% Cmax} (h) | 4.2 | 5.6 | 2.0 | 1.8 | 3.1 | 3.4 | 1.6 |
| Fᵣₑₗ (%) | 95% | 30% | 127% | 144% | 95% | 98% | 39% |

TABLE 22
Pharmacokinetics of donepezil after oral dosing
of 1 mg donepezil equivalents of the glutaryl
(S) valinamide ester prodrug/kg to male dogs

| Pharmacokinetic | | | | | | | |
|---|---|---|---|---|---|---|---|
| parameter | 205 | 207 | 209 | 211 | 213 | Mean | sd |
| Cₘₐₓ (ng/mL) | 26 | 17 | 9 | 5 | 2 | 12 | 9.8 |
| Tₘₐₓ (h) | 1.0 | 1.0 | 1.0 | 1.0 | 1.0 | 1.0 | (1-1) |
| AUCt (ng · h/mL) | 175 | 124 | 46 | 19 | 6 | 74 | 73 |
| AUC (ng · h/mL) | 182 | 128 | 48 | 20 | 7 | 77 | 75 |
| t½ (h) | 5.2 | 4.4 | 5.0 | 2.5 | 2.1 | 3.8 | 1.4 |
| T_{>50% Cmax} (h) | 4.9 | 5.5 | 3.1 | 2.4 | 2.5 | 3.7 | 1.4 |
| Fᵣₑₗ (%) | 85% | 25% | 62% | 44% | 28% | 49% | 23% |

These results confirm the potential of these prodrugs to deliver significant levels of the active drug (donepezil) to the systemic circulation of the beagle dog.

Monkeys

Methods

Using a three way crossover design a group of five male cynomolgus dogs was orally dosed with either donepezil itself or its glutaryl enol ester or glutaryl valinamide enol ester prodrug. Serial blood samples (0.5 mL) were subsequently collected from a suitable vein over 24 h, into tubes containing 1.75 mg/mL K₃EDTA, and 25 μL of 1M citrate buffer pH 3.0 with 20 mM 1,4-saccharolactone; acting as a pro-drug stabiliser and β-glucuronidase inhibitor, respectively to prevent post collection enzymic hydrolysis of the prodrug. Separated plasma samples were subjected to bioanalysis for both the prodrugs and donepezil itself using a qualified LC/MS-MS bioanalytical method. Subsequent pharmacokinetic analysis was then undertaken using Win NonLin to generate the relevant PK parameters including Cₘₐₓ, Tₘₐₓ and AUC and T_{1/2}.

Results—These are shown in Tables 23, 124, & 25.

TABLE 23
Pharmacokinetics of donepezil after oral dosing of 1
mg donepezil free base equivalents/kg to male monkeys

| Pharmacokinetic | | | | | | | |
|---|---|---|---|---|---|---|---|
| parameter | 713 | 715 | 717 | 719 | 721 | Mean | sd |
| Cₘₐₓ (ng/mL) | 4 | 3 | 12 | 2 | 4 | 5 | 4 |
| Tₘₐₓ (h) | 4 | 1.0 | 1.0 | 2.0 | 2.0 | 1.0 | (1-2) |
| AUC (ng · h/mL) | 11 | 12 | 48 | 8 | 15 | 19 | 17 |
| t½ (h) | 12 | 2.5 | 2.5 | 2.4 | 2.5 | 2.2 | 0.5 |
| T_{>50% Cmax} (h) | 1.4 | 3.0 | 2.8 | 3.7 | 2.9 | 2.8 | 0.7 |

TABLE 24
Pharmacokinetics of donepezil after oral dosing of 1 mg donepezil
equivalents of the glutaryl enol ester prodrug/kg to male monkeys

| Pharmacokinetic | | | | | | | |
|---|---|---|---|---|---|---|---|
| parameter | 713 | 715 | 717 | 719 | 721 | Mean | sd |
| Cₘₐₓ (ng/mL) | 4 | 10 | 15 | 2 | 5 | 7 | 5 |
| Tₘₐₓ (h) | 1.0 | 0.5 | 2.0 | 2.0 | 2.0 | 2.0 | (0.5-2) |
| AUC (ng · h/mL) | 18 | 27 | 68 | 11 | 23 | 29 | 23 |
| t½ (h) | 2.1 | 2.4 | 4.2 | 2.7 | 2.5 | 2.8 | 0.9 |
| T_{>50% Cmax} (h) | 3.6 | 1.6 | 3.3 | 4.1 | 3.2 | 3.2 | 0.9 |
| Fᵣₑₗ (%) | 152% | 219% | 141% | 145% | 156% | 162% | 29% |

TABLE 25
Pharmacokinetics of donepezil after oral dosing
of 1 mg donepezil equivalents of the glutaryl (S)
valinamide ester prodrug/kg to male monkeys

| Pharmacokinetic | | | | | | | |
|---|---|---|---|---|---|---|---|
| parameter | 713 | 715 | 717 | 719 | 721 | Mean | sd |
| Cₘₐₓ (ng/mL) | 3 | 1 | 12 | 1 | 3 | 4 | 4 |
| Tₘₐₓ (h) | 2.0 | 2.0 | 2.0 | 2.0 | 2.0 | 2.0 | (2-2) |
| AUC (ng · h/mL) | 11 | 5 | 48 | 5 | 13 | 16 | 18 |
| t½ (h) | 1.6 | 1.7 | 2.5 | 2.0 | 2.3 | 2.0 | 0.4 |
| T_{>50% Cmax} (h) | 4.0 | 3.8 | 3.0 | 3.1 | 2.8 | 3.3 | 0.5 |
| Fᵣₑₗ (%) | 97% | 44% | 99% | 64% | 89% | 79% | 21% |

These results confirm the potential of these prodrugs to deliver significant levels of the active drug (donepezil) to the systemic circulation of the cynomolgus monkey.

### Example 19

### Synthesis of Selected Piroxicam Prodrugs

In order to minimise the possibility of a direct irritant effect of this drug on the gastric and intestinal mucosa—and the possibility of resultant ulceration—the drug was transiently inactivated by making the corresponding succinyl valine and glutaryl PABA prodrugs in the manner described below (scheme 17).

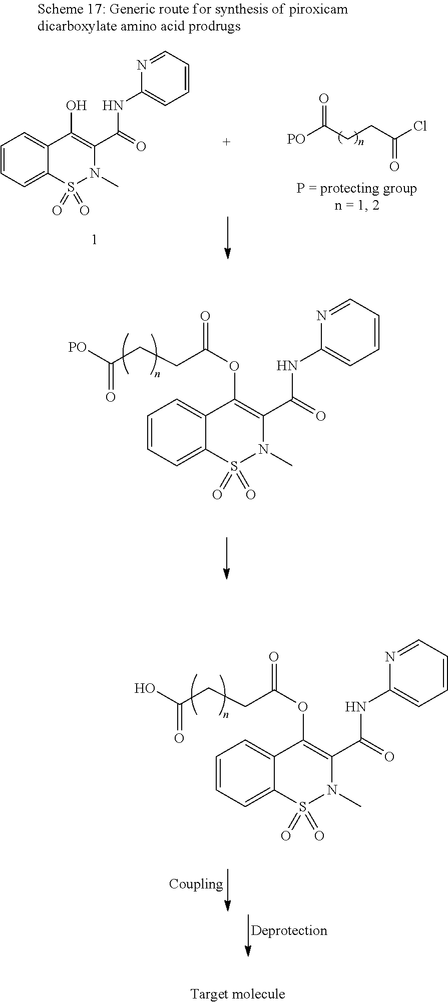

### Example 20

### Synthesis of Selected Velefaxine Prodrugs

Up to 30% of patients treated with venlefaxine experience nausea which can be a major deterent to treatment continuation. Transient inactivation of the drug while in the gut lumen would avoid the locally mediated interaction with 5HT receptors in the gut and so minimize this risk of nausea. This can be achieved by making the prodrugs described below.

The synthesis of venelefaxine succinyl valine amide, shown below, was untaken as shown in scheme 18

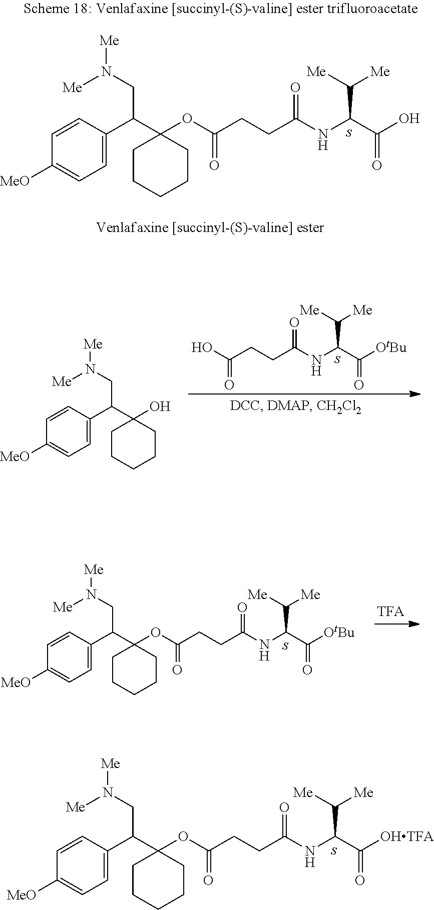

Venlafaxine free base was coupled with succinyl-(S)-valine tert-butyl ester via a N,N′-dicyclohexylcarbodi-imide (DCC) mediated reaction to give venlafaxine[succinyl-(S)-valine tert-butyl ester]ester. The tert-butyl ester was cleaved using trifluoroacetic acid to give, after column purification, venlafaxine[succinyl-(S)-valine]ester trifluoroacetate.

Detail

To a stirred solution of venlafaxine free base (0.44 g, 1.59 mmol) in dichloromethane (10 mL) was added a solution of succinyl-(S)-valine tert-butyl ester (0.57 g, 2.06 mmol) in dichloromethane (50 mL), N,N-dimethylaminopyridine (4 mg) and N,N′-dicyclohexylcarbodi-imide (0.46 g, 2.23 mmol) and the reaction mixture was stirred overnight. The resulting suspension was filtered through Celite and the filtrate concentrated to afford an oil which was purified by medium-pressure chromatography on silica eluting with a gradient of 0→10% methanol in dichloromethane to give a crude solid (0.77 g) which was further purified using a Biotage Isolera automated chromatography system under normal phase conditions (silica column, gradient of 0→20% methanol in dichloromethane) with detection at 274 nm to afford venlafaxine [succinyl-(S)-valine tert-butyl ester]ester (0.48 g, 57%), as a clear oil.

R_{f} 0.42 (methanol-dichloromethane, 1:9 v/v).

Venlafaxine[succinyl-(S)-valine tert-butyl ester]ester (0.48 g, 0.88 mmol) in trifluoroacetic acid (10 mL) was stirred at room temperature for 45 min. The mixture was evaporated to dryness and residual trifluoroacetic acid was removed azeotropically with chloroform (5×35 mL) to afford an oil which was purified on a Biotage Isolera automated chromatography system under reversed-phase conditions (C₁₈ column, gradient of 0→100% MeCN in 0.02% aqueous HCl) with detection at 254 nm to give venlafaxine [succinyl-(S)-valine]ester trifluoroacetate (0.16 g, 32%), as a colourless oil.

The synthesis of venelefaxine glutaryl para amino benzoic acid (PABA) amide, shown below, was undertaken as depicted in scheme 19.

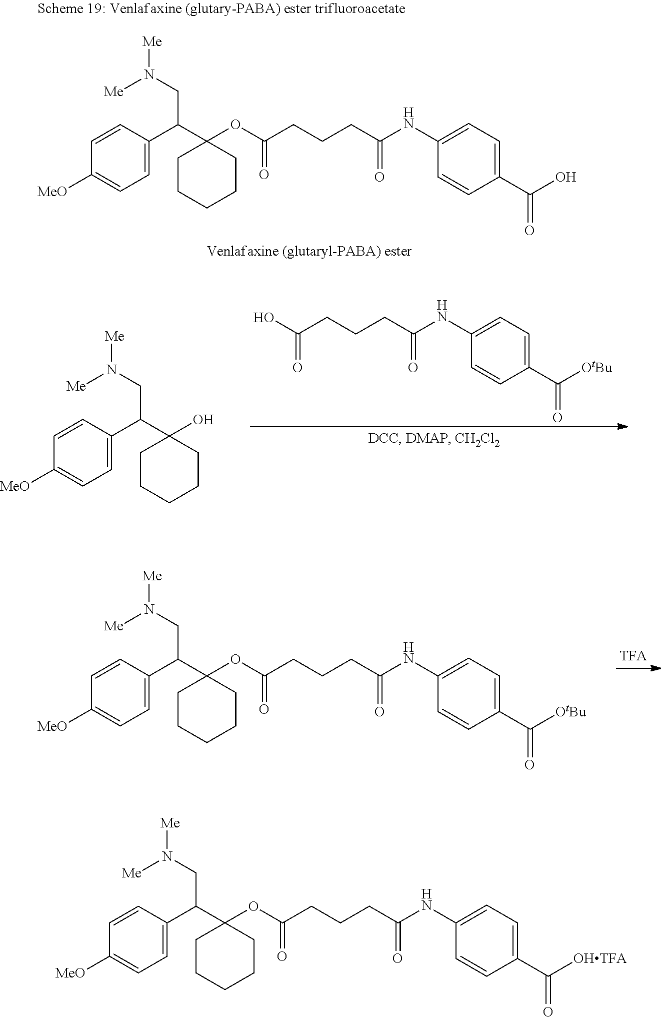

### Example 21

### Synthesis of Selected Pazanib Prodrugs

Temporary inactivation of pazapanib while the drug is resident in the GI lumen should reduce the pronounced diarrhoea associated with this drug which is currently a serious deterrent to patient compliance with >50% patients so affected.

The synthesis of pazapanib succinyl valine amide, shown below, was undertaken as depicted in scheme 20

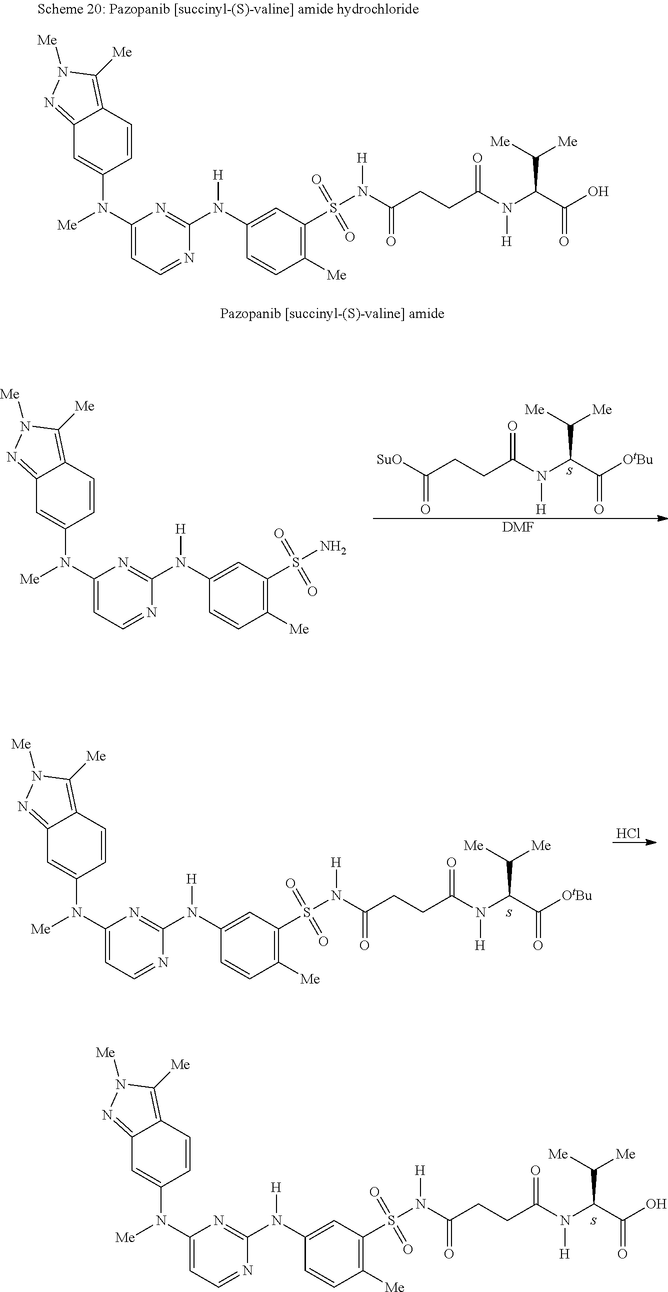

The synthesis of pazapanib glutaryl para amino benzoic acid (PABA) amide, shown below, was undertaken as depected in scheme 21

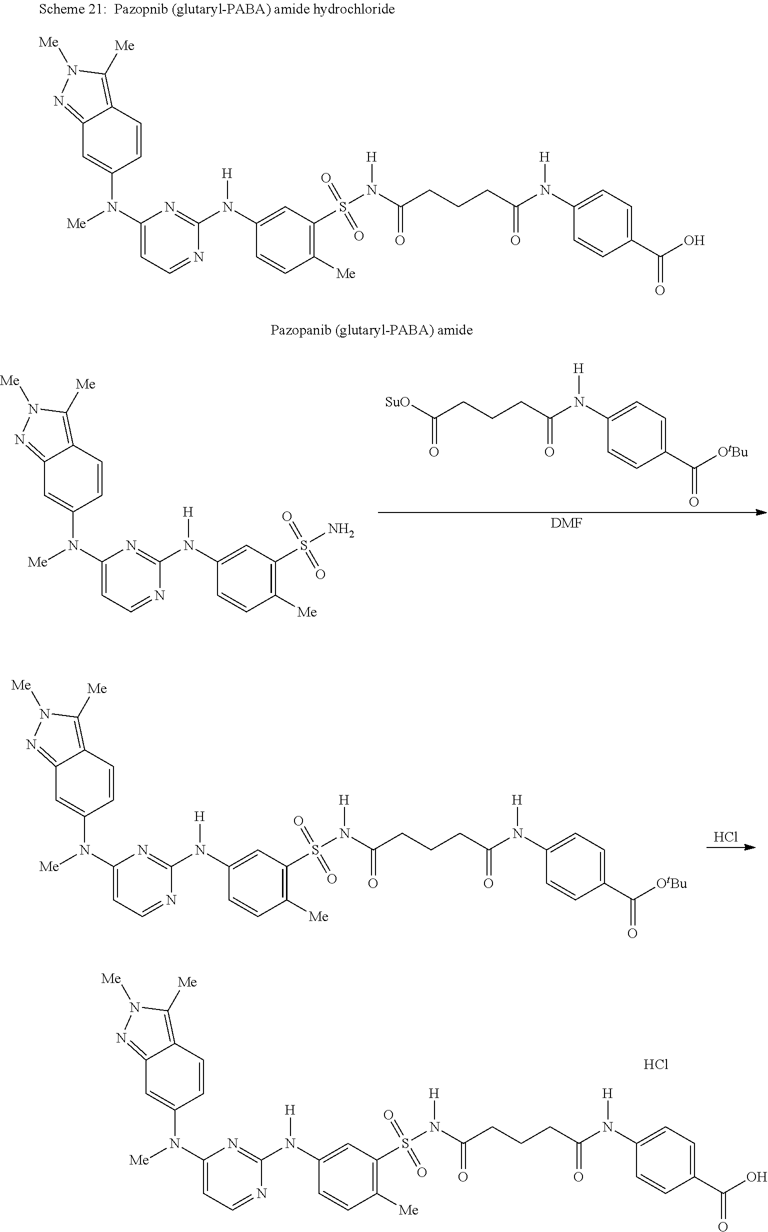

### Example 22

### Synthesis of Selected Alendronate Prodrugs

Alendronate induced oesophageal ulceration could be avoided by ensuring that prior to absorption the drug is inactivated while it is present in the GI tract. To this end the dicarboxylate amino acid conjugates can be made as outlined below in scheme 22 below:—

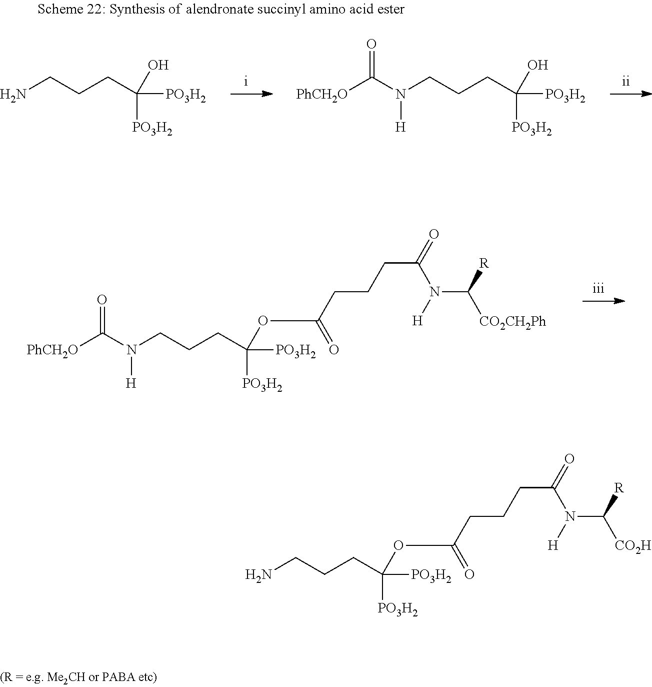

Reagents and Solvents: i, PhCH₂OCOCl, aq NaOH; ii, PhCH₂OCOCH′PrNHCO(CH₂)₃COF, aq NaOH; iii, Pd/H₂, THF.

### Example 23

### Synthesis of Selected Amprenavir Prodrugs

Transient inactivation of amprenavir should avoid the direct adverse effects of this drug on the gut epithelium and so improve patient compliance and hence therapeutic benefit.

The synthesis of amprenavir succinyl valine amide, shown below, was untaken as depicted in scheme 23

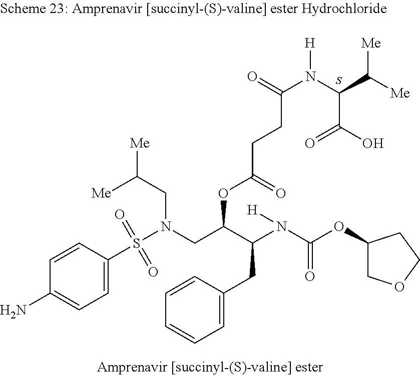

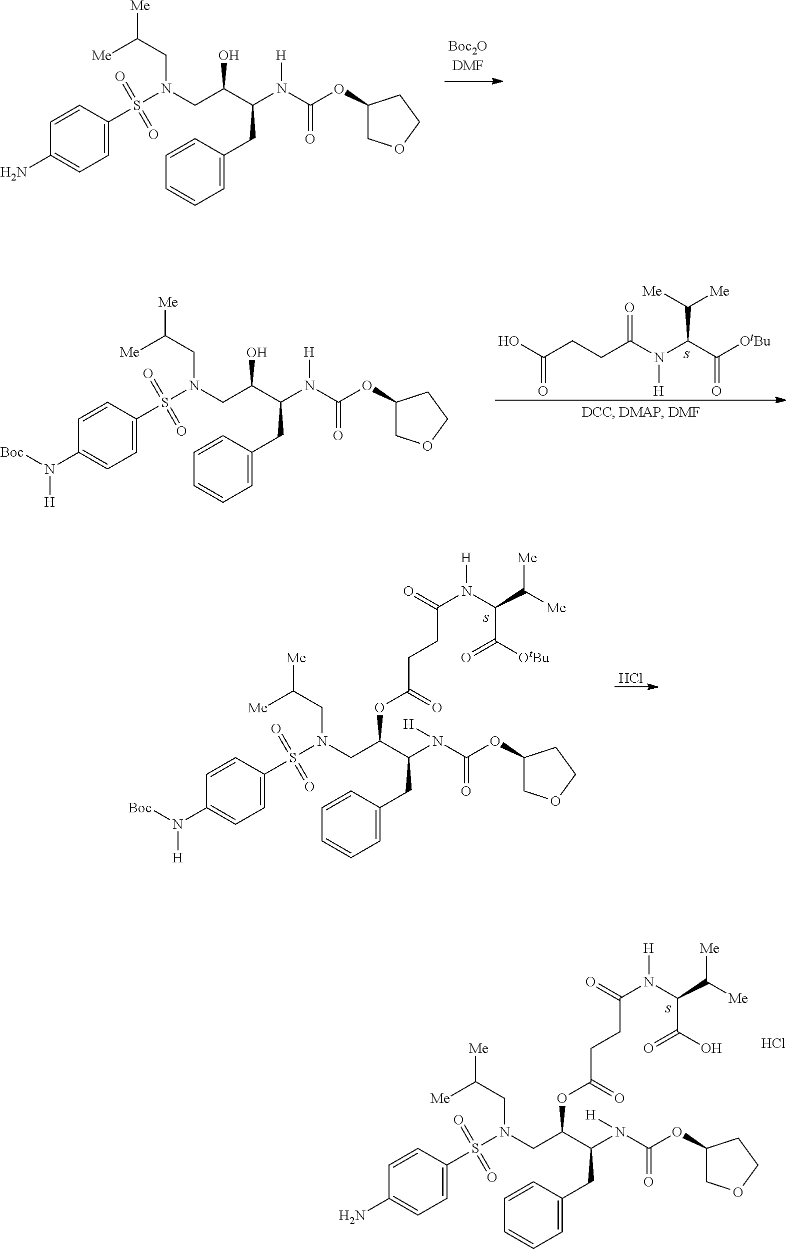

The synthesis of amprenavir glutaryl para amino benzoic acid (PABA) amide, shown below, was undertaken as depicted in scheme 24

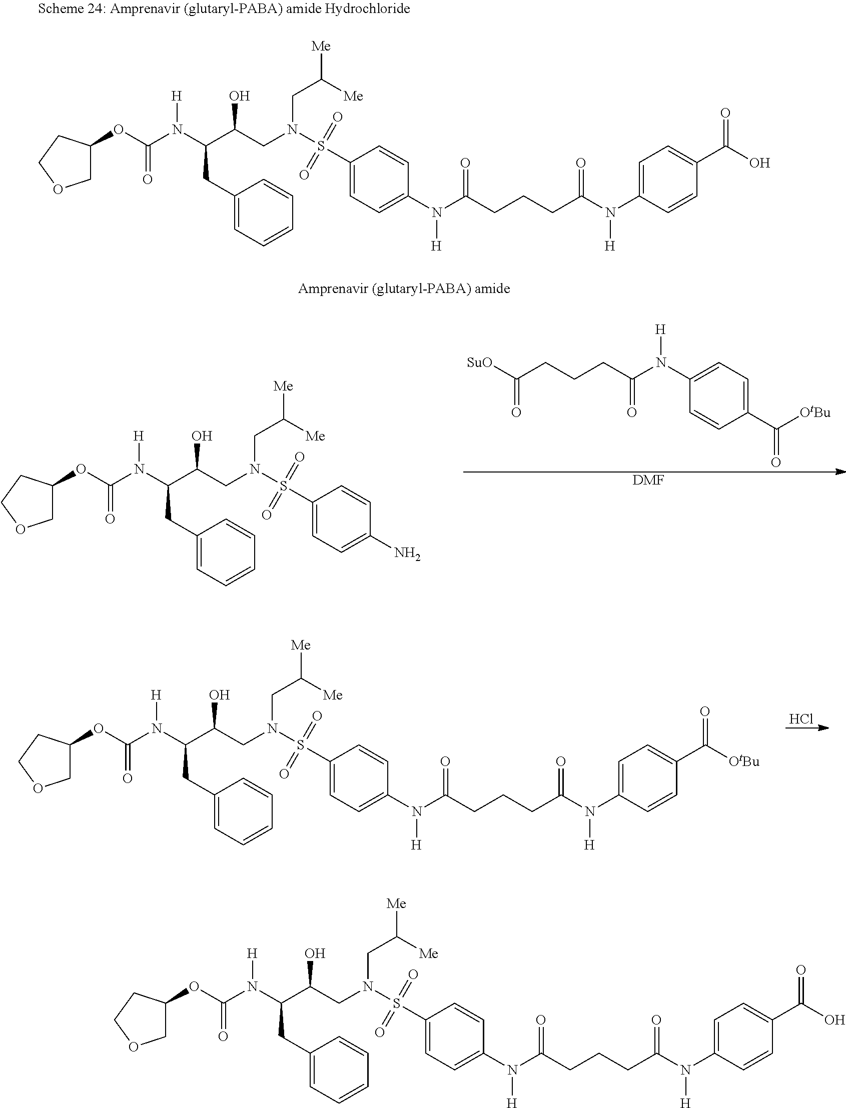

### Example 24

### Synthesis of Didanosine[succinyl-(S)-valine]ester

The synthesis of didanosine[succinyl-(S)-valine]ester was achieved in two steps (Scheme 25). Succinyl-(S)-valine benzyl ester was reacted with didanosine using N,N′-dicyclohexylcarbodiimide (DCC) as coupling agent to afford didanosine[succinyl-(S)-valine benzyl ester]ester, following purification by normal phase chromatography.

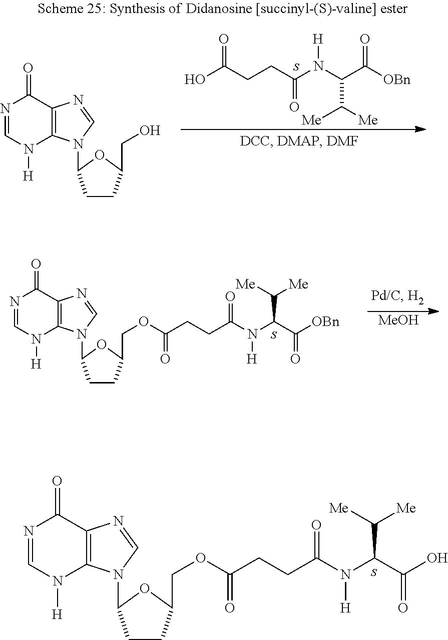

Removal of the benzyl group was achieved by palladium catalysed hydrogenation to give the required didanosine[succinyl-(S)-valine]ester as a white solid.

Detail

To a stirred solution of didanosine (2′,3′-dideoxyinosine) (0.31 g, 1.33 mmol), succinyl-(S)-valine benzyl ester (0.41 g, 1.33 mmol) and N,N-dimethylaminopyridine (16 mg, 0.13 mmol) in dry DMF (8 mL) at 0° C. was added N,N-dicyclohexylcarbodi-imide (0.29 g, 1.39 mmol) in one portion and stirring was then continued overnight during which time the mixture was allowed to warm to room temperature. The resulting suspension was filtered through Celite and the filtrate was poured into water (50 mL), and extracted with ethyl acetate (3×50 mL). The combined organic layers were washed with water (3×50 mL) and saturated brine (50 mL), dried (MgSO₄) and concentrated to give a white semi-solid (0.61 g). This crude material was purified using a Biotage Isolera automated chromatography system under normal phase conditions (silica column, gradient of 0→25% ethyl acetate in petrol) with detection at 254 nm to afford didanosine[succinyl-(S)-valine benzyl ester]ester (92 mg, 13%), as a white solid.

10% Palladium on carbon (40 mg) was cautiously wetted with anhydrous ethanol (1 mL) under a nitrogen atmosphere. A solution of didanosine[succinyl-(S)-valine benzyl ester]ester (88 mg, 0.17 mmol) in methanol (2 mL) was added and the flask was evacuated. An atmosphere of hydrogen was introduced via a balloon and the mixture was stirred for 4 h at room temperature. The catalyst was removed by filtration of the suspension through a thin layer of Celite and the filtrate was concentrated to yield didanosine [succinyl-(S)-valine]ester (71 mg, 97%), as a white solid.

### Example 26

### Synthesis of Selected Meformin Prodrugs

The temporary inactivation of metformin while the drug is in the gut should avoid the potential for directly mediated intestinal hurry associated with this drug.

The synthesis of metformin succinyl valine amide, shown below, was untaken as depicted in scheme 26:

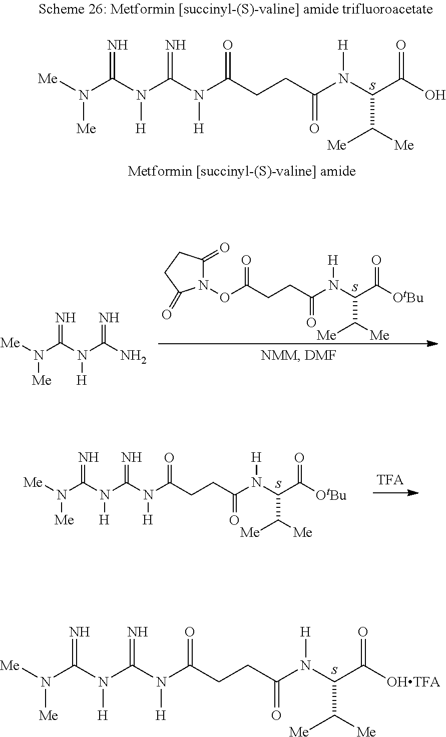

The synthesis of metformin succinyl valine amide, shown below, was untaken as depicted in scheme 27

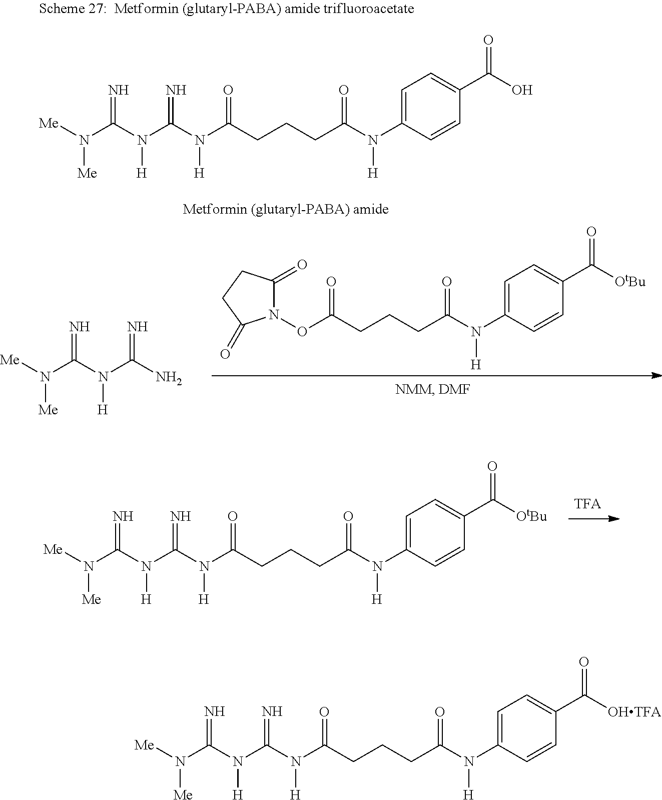

### Example 27

### Synthesis of acetazolamide[succinyl-(S)-valine]amide

Amongst the side effects of acetazolamide treatment are GI disturbances believed to be due a direct local action within the gut lumen. Transiently inactivation the drug as described below should enable these unwanted side-effects to be overcome.

The synthesis of acetazolamide[succinyl-(S)-valine]amide was achieved in two reaction steps (Scheme 28). Succinyl-(S)-valine tert-butyl ester was reacted with acetazolamide using 1-(3-dimethylaminopropyl)-3-ethylcarbodi-imide hydrochloride (EDCI) as coupling agent to afford acetazolamide[succinyl-(S)-valine-tert-butyl ester]amide, following purification by normal phase chromatography.

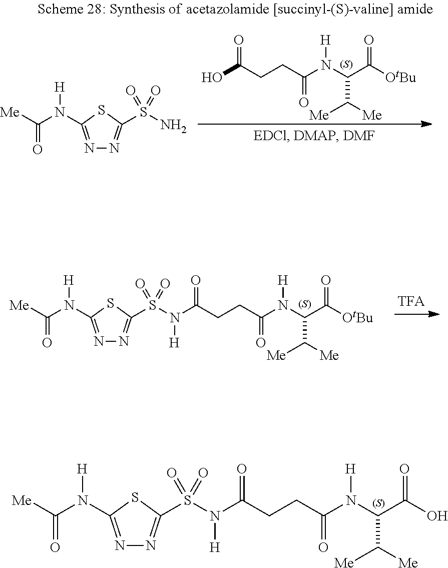

Removal of the tert-butyl group was achieved using trifluoroacetic acid to give the required acetazolamide[succinyl-(S)-valine]amide as an off-white solid.

Detail

To a stirred solution of succinyl-(S)-valine tert-butyl ester (0.50 g, 1.83 mmol), acetazolamide (0.45 g, 2.01 mmol) and N,N-dimethylaminopyridine (22 mg, 0.18 mmol) in dry DMF (15 mL) at 0° C. was added 1-(3-dimethylaminopropyl)-3-ethylcarbodiimide hydrochloride (0.39 g, 2.01 mmol) in one portion and stirring was then continued overnight at 70° C. The resulting solution was allowed to cool to room temperature, poured into water (100 mL) and extracted with ethyl acetate (3×50 mL). The combined organic layers were washed with water (3×50 mL) and saturated brine (50 mL), dried (MgSO₄) and concentrated to give a white solid (0.39 g). The crude solid was purified using a Biotage Isolera automated chromatography system under normal phase conditions (silica column, gradient of 0→25% methanol in dichloromethane) with detection at 254 nm to afford acetazolamide[succinyl-(S)-valine-tert-butyl ester]amide (0.14 g, 16%), as a white solid.

Acetazolamide[succinyl-(S)-valine-tert-butyl ester]amide (94 mg, 0.20 mmol) in trifluoroacetic acid (10 mL) was stirred at room temperature for 30 min. The mixture was evaporated to dryness and residual trifluoroacetic acid was removed azeotropically using chloroform (4×20 mL). The crude residual solid was triturated with diethyl ether, collected by suction filtration and dried in vacuo to afford acetazolamide[succinyl-(S)-valine]amide (90 mg, quantitative), as an off-white solid.

### Example 28

### Synthesis of acetazolamide[succinyl-PABA]amide

The synthesis of acetazolamide (glutaryl-PABA) amide was achieved in two steps (Scheme 29). Glutaryl-PABA tert-butyl ester was reacted with acetazolamide using 1-(3-dimethylaminopropyl)-3-ethylcarbodi-imide hydrochloride (EDCI) as coupling agent to afford acetazolamide (glutaryl-PABA tert-butyl ester) amide, after purification by normal phase chromatography.

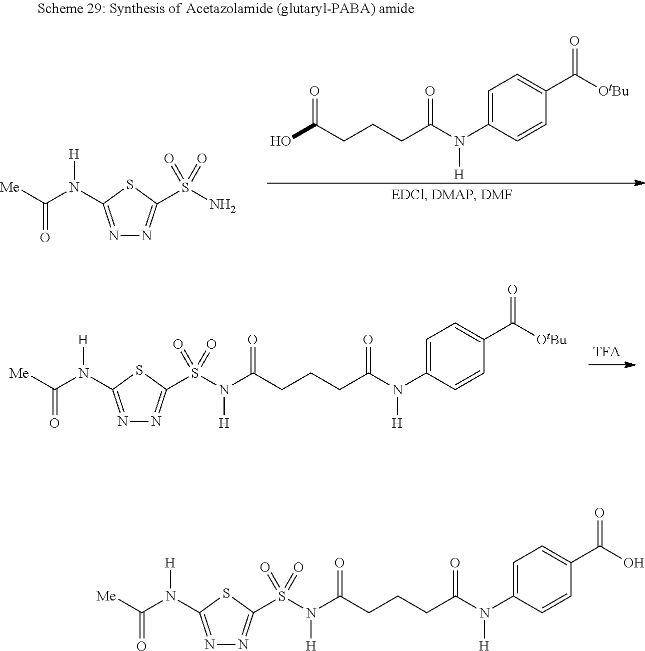

Removal of the tert-butyl group was achieved by treatment with trifluoroacetic acid to give the required acetazolamide (glutaryl-PABA) amide as a pale yellow solid.

Detail

To a stirred solution of glutaryl-PABA tert-butyl ester (0.61 g, 2.00 mmol), acetazolamide (0.49 g, 2.20 mmol) and N,N-dimethylaminopyridine (25 mg, 0.20 mmol) in dry DMF (15 mL) at 0° C. was added 1-(3-dimethylaminopropyl)-3-ethylcarbodiimide hydrochloride (0.42 g, 2.20 mmol) in one portion and stirring was then continued overnight at room temperature. The resulting solution was poured into water (60 mL) and extracted with ethyl acetate (3×50 mL). The combined organic extracts were washed with water (3×50 mL) and saturated brine (50 mL), dried (MgSO₄) and concentrated to give a white solid (0.73 g). The crude solid was purified using a Biotage Isolera automated chromatography system under normal phase conditions (silica column, gradient of 0→25% methanol in dichloromethane) with detection at 254 nm to afford acetazolamide (glutaryl-PABA tert-butyl ester) amide (0.27 g, 27%) as a white solid.

Acetazolamide (glutaryl-PABA tert-butyl ester) amide (0.24 g, 0.47 mmol) in trifluoroacetic acid (20 mL) was stirred at room temperature for 30 min. The mixture was evaporated to dryness and residual trifluoroacetic acid was removed azeotropically with chloroform (4×20 mL). The residue was triturated with diethyl ether, collected by suction filtration and dried in vacuo to give acetazolamide (glutaryl-PABA) amide (0.20 g, 94%), as a pale yellow solid

### Example 29

### Additional Antibiotic Prodrugs—Synthetic Schemes

A. Ceftaroline

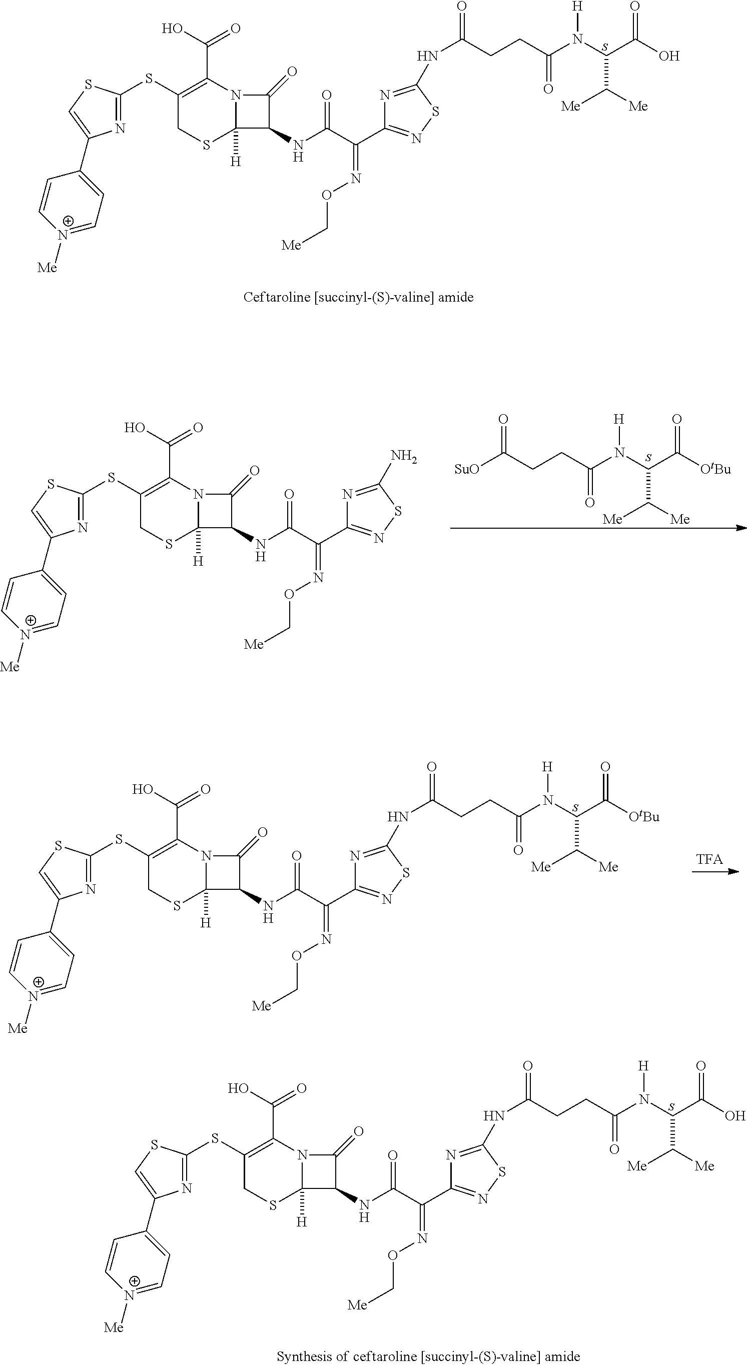

B. Ceftobiprole

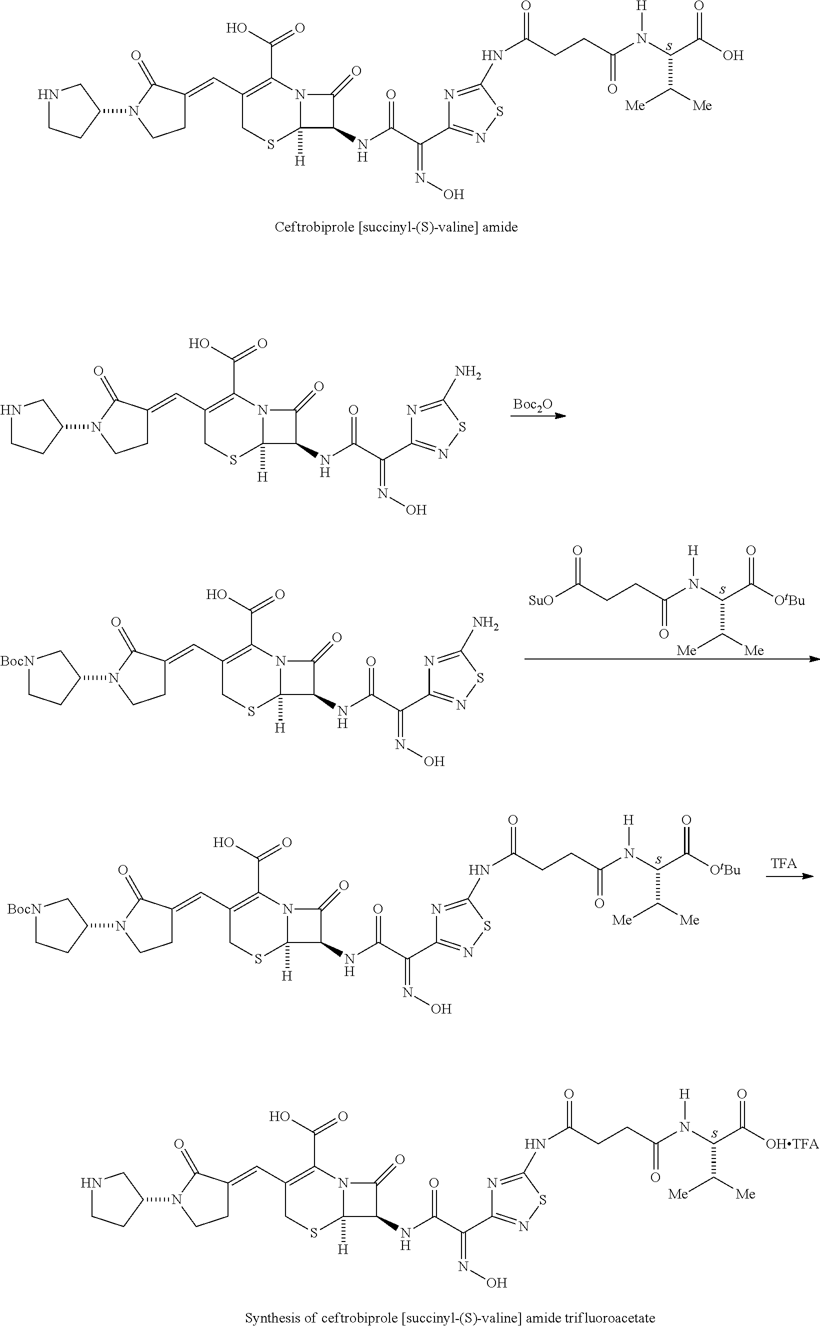

C. Ceftriaxone

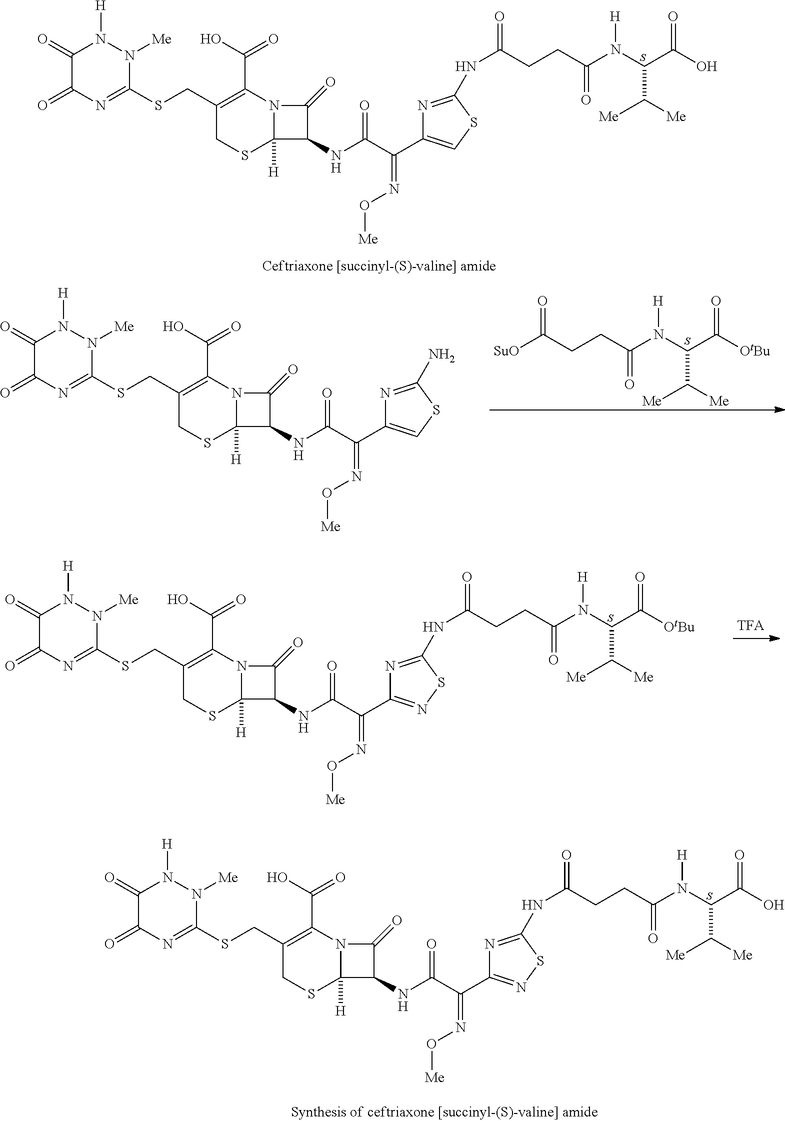

D. Trovafloxacin

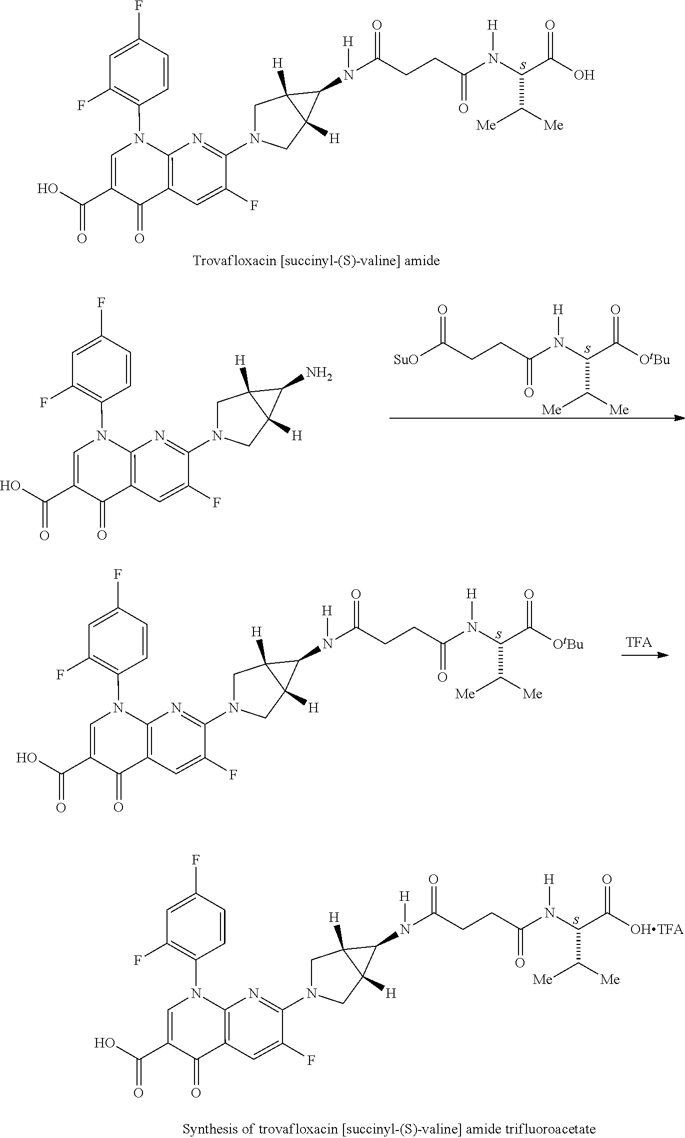

Patents, published patent applications, and non-patent publications cited herein are hereby incorporated by reference in their entirety.

1. A prodrug for treating a systemic disorder, the prodrug having a structure of Formula (I) or a pharmaceutically acceptable salt thereof:
D-L-R (I)
wherein:
D- is a non-opioid drug having a free hydroxyl group, a free amine group or an enolisable carbonyl group, the drug being for use in treating said systemic disorder;
—R is an amino acid residue containing from 2 to 20 carbon atoms or a peptide formed from 2 to 10 independently selected amino acids each containing from 2 to 20 carbon atoms; or —R is an amino amide residue containing from 2 to 20 carbon atoms and terminating with a —CONR^{a}R^{b} group, or —R is a peptide formed from 2 to 9 independently selected amino acids each containing from 2 to 20 carbon atoms and terminating with an amino amide residue containing from 2 to 20 carbon atoms, the amino amide residue terminating with a —CONR^{a}R^{b} group;
R^{a} and R^{b} when present are each independently selected from the group consisting of: H, C₁₋₆ alkyl, —(CH₂)ᵣ—C₃₋₆ cycloalkyl, phenyl and benzyl, or wherein R^{a} and R^{b} together with the nitrogen atom to which they are attached form a ring containing 3, 4, 5 or 6 carbon atoms; wherein each of the R^{a} and
R^{b} groups may be unsubstituted or substituted with 1 or 2 substituent groups independently selected at each occurrence from the group consisting of: F, Cl, CN and OH; r is an integer of 0 or 1;
-L- is 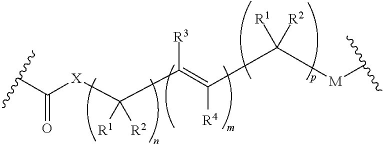
R¹ and R² are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷; or together with the atom to which they are bonded, R¹ and R² may form a carbonyl, an ethylene or a C₃₋₆ cycloalkyl; and R³ and R⁴ are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷;
R⁵ and R⁶ are each independently selected from the group consisting of: H, C₁₋₆ alkyl, C₁₋₆ haloalkyl, C₃₋₈ cycloalkyl and phenyl;
R⁷ is selected from the group consisting of: hydroxyl, C₁₋₆ alkyl, C₁₋₆ alkoxy, C₃₋₈ cycloalkyl and phenyl;
X is selected from the group consisting of: a bond, —O—, —NH—, and a saturated or unsaturated ring having from 3 to 6 carbon atoms in the ring;
M is selected from the group consisting of: 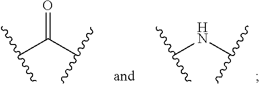
n and p are each independently an integer of 0-16, provided that the sum of n and p is an integer of 0-16;
m is an integer of 0-2; and
q is an integer of 0-3.

2. A prodrug for treating a systemic disorder, the prodrug having a structure of Formula (II) or a pharmaceutically acceptable salt thereof:
D-L-R (II)
wherein:
D- is a non-opioid drug having a free hydroxyl group, a free amine group or an enolisable carbonyl group, the drug being for use in treating said systemic disorder;
—R is —OR^{a} or —NR^{a}R^{b};
R^{a} and R^{b} are each independently selected from the group consisting of: H, C₁₋₆ alkyl, —(CH₂)ᵣ—C₃₋₆ cycloalkyl, phenyl and benzyl, or wherein R^{a} and R^{b} together with the nitrogen atom to which they are attached form a ring containing 3, 4, 5 or 6 carbon atoms; wherein each of the R^{a} and R^{b} groups may be unsubstituted or substituted with 1 or 2 substituent groups independently selected at each occurrence from the group consisting of: F, Cl, CN and OH; r is an integer of 0 or 1;
-L- is 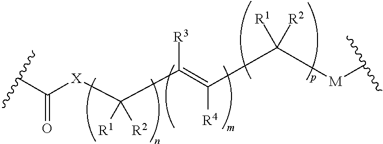
R¹ and R² are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷; or together with the atom to which they are bonded, R¹ and R² may form a carbonyl, an ethylene or a C₃₋₆ cycloalkyl; and
R³ and R⁴ are each independently selected at each occurrence from the group comprising: hydrogen, halogen, hydroxy, C₁₋₆ alkoxy, C₁₋₆ alkyl C₁₋₆ alkoxy, —(CR⁵R⁶)_{q}OC(═O)R⁷, —(CR⁵R⁶)_{q}C(═O)R⁷, —C(═O)R⁷, C₁₋₆ alkyl, C₁₋₆ haloalkyl, aryl, —NR⁵R⁶ and —NR⁵(CO)R⁷;
R⁵ and R⁶ are each independently selected from the group consisting of: H, C₁₋₆ alkyl, C₁₋₆ haloalkyl, C₃₋₈ cycloalkyl and phenyl;
R⁷ is selected from the group consisting of: hydroxyl, C₁₋₆ alkyl, C₁₋₆ alkoxy, C₃₋₈ cycloalkyl and phenyl;
X is selected from the group consisting of: a bond, —O—, —NH—, and a saturated or unsaturated ring having from 3 to 6 carbon atoms in the ring;
M is selected from the group consisting of: 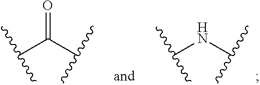
n and p are each independently an integer of 0-16, provided that the sum of n and p is an integer of 0-16;
m is an integer of 0-2; and
q is an integer of 0-3.

3. The prodrug of claim 1 wherein D is selected from the group consisting of: metronidazole, 2-, 3- or 4-clindamycin, lincomycin, ampicillin, amoxicillin, clinafloxacin, delafloxacin, gemifloxacin, nadifloxacin, pazufloxacin, sitafloxacin, sparfloxacin, trovafloxacin, tosufloxacin, ceftobiprole (active metabolite of ceftobiprole medocaril), ceftaroline (active metabolite of ceftaroline fosamil) cefixime, ceftriaxone, cefoperazone, cefotaxime, cefadroxil, donepezil, memantine, miglustat, eliglustat, venlafaxine, desvenlafaxine, fluvoxamine, milnacipran, alendronate, etidronate, pamidronate, neridronate, olpadronate, ibandronate, zoledronate, pazopanib, thioguanine, melphelan, hydroxyurea, temozolomide, metformin, amprenavir, saquinavir, ritonavir, indinavir, nelfinavir, lopinavir, atazanavir, tipranavir, darunavir, didanosine, propafenone, piroxicam, meloxicam, tenoxicam, lornoxicam, acetazolamide ceftazidime, ciprofloxacin and levofloxacin.

4. The prodrug of claim 1 wherein the systemic disease treatable using a prodrug of Formula I includes a disorder selected from the group consisting of: a bacterial infection, an arrhythmia, Gauchers disease, Alzheimer's disease, rheumatoid arthritis, depression, cancer, osteoporosis, a viral infection and diabetes.

5. A prodrug having a structure of Formula (I) or Formula (II) or a pharmaceutically acceptable salt thereof as defined in claim 1, wherein: D- is a drug selected from the group consisting of:—
metronidazole, 2-, 3- or 4-clindamycin, lincamycin, ampicillin, amoxicillin, clinafloxacin, delafloxacin, gemifloxacin, nadifloxacin, pazufloxacin, sitafloxacin, sparfloxacin, trovafloxacin, tosufloxacin, ceftabiprole (active metabolite of ceftabiprole medocaril), ceftaroline (active metabolite of ceftaroline fosamil) cefixime, ceftriaxone, cefoperazone, cefotaxime, cefadroxil, donepezil, memantine, miglustat, eliglustat, venlafaxine, desvenlafaxine, fluvoxamine, milnacipran, alendronate, etidronate, pamidronate, neridronate, olpadronate, ibandronate, zoledronate, pazopanib, thioguanine, melphelan, hydroxyurea, temozolomide, metformin, amprenavir, saquinavir, ritonavir, indinavir, nelfinavir, lopinavir, atazanavir, tipranavir, darunavir, didanosine, propafenone and piroxicam, meloxicam, tenoxicam and lornoxicam, acetazolamide, ceftazidime, ciprofloxacin and levo floxacin.

6. The prodrug of any preceding claim wherein M is

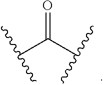

7. The prodrug of claim 1 wherein X is a bond.

8. The prodrug of claim 1 wherein R¹ and R² are each independently selected from the group consisting of: hydrogen, hydroxy, —(CR⁵R⁶)_{q}C(═O)R⁷, —C(═O)R⁷, C₁₋₆ alkyl, aryl, —NR⁵R⁶ and —NR⁵(CO)R⁷; or together with the atom to which they are bonded, R¹ and R² may form a carbonyl or an ethylene.

9. The prodrug of claim 8 wherein R¹ and R² are each independently selected from the group consisting of: hydrogen, hydroxy, —(CH₂)C(═O)OH, —C(═O)OH, methyl and —NH₂.

10. The prodrug of claim 8 wherein R¹ and R² are each independently selected from the group consisting of: hydrogen and C₁₋₄ alkyl.

11. The prodrug of claim 1 wherein R³ and R⁴ are each independently selected from the group consisting of: hydrogen, C₁₋₆ alkoxy, —C(═O)R⁷ and C₁₋₆ alkyl.

12. The prodrug of claim 1 wherein R⁵ is H.

13. The prodrug of claim 1 wherein R⁶ is H.

14. The prodrug of claim 1 wherein R⁷ is selected from the group consisting of: hydroxyl and C₁₋₆ alkyl.

15. The prodrug of claim 1 wherein n is 2 or 3.

16. The prodrug of claim 1 wherein m is 0.

17. The prodrug of claim 1 wherein p is 0.

18. The prodrug of claim 1 wherein L is a residue selected from the group consisting of:

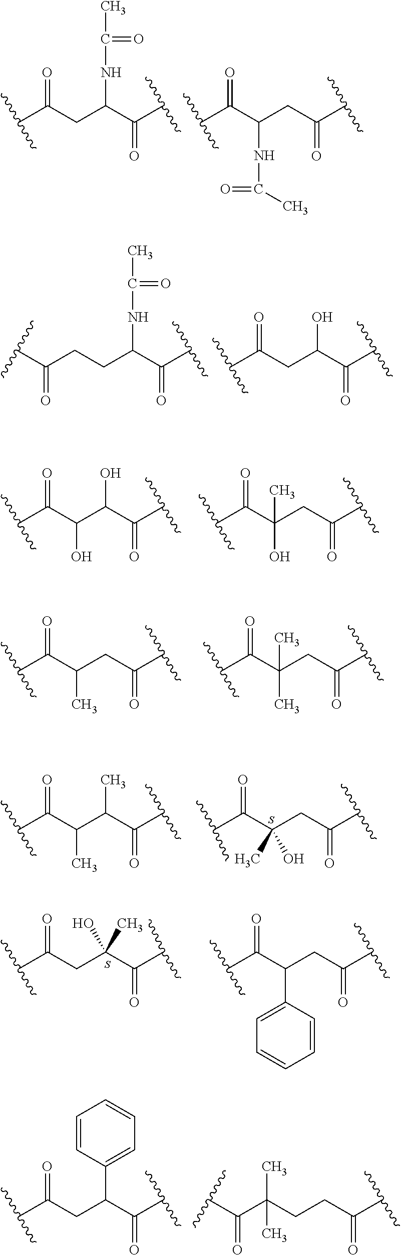

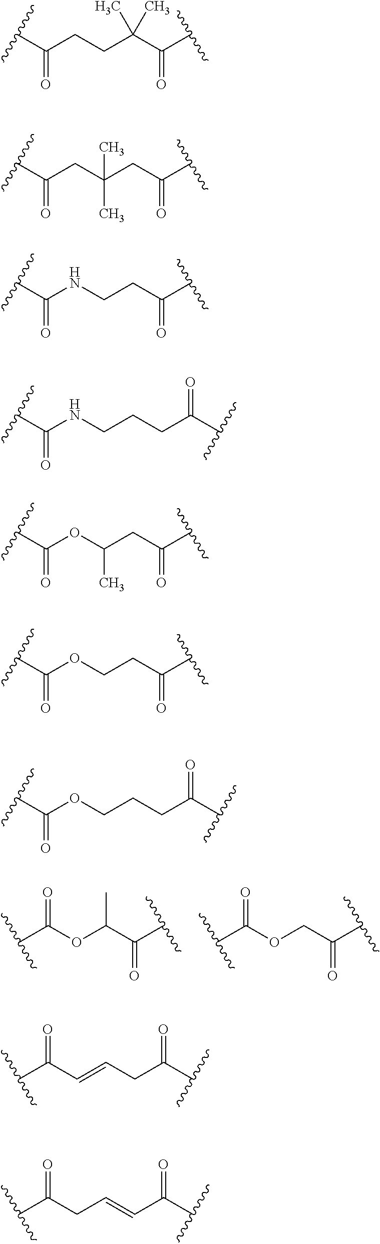

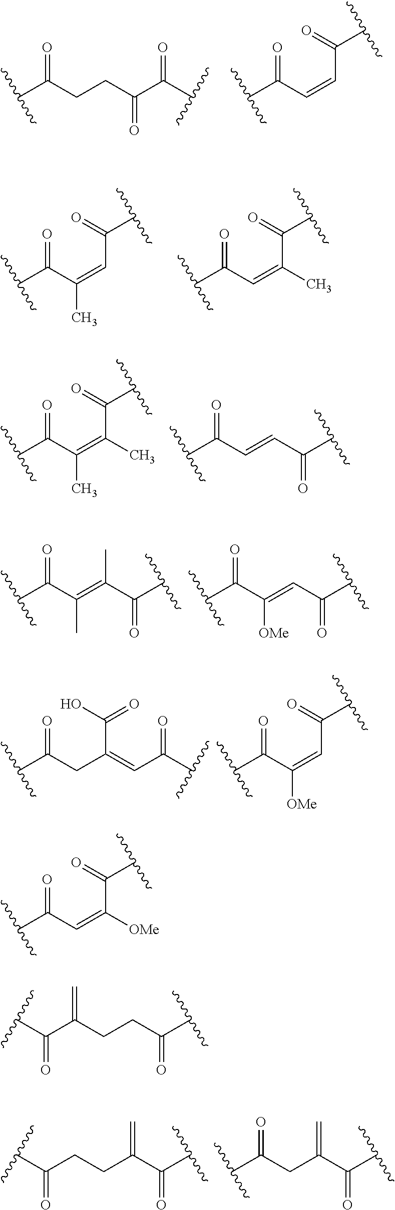

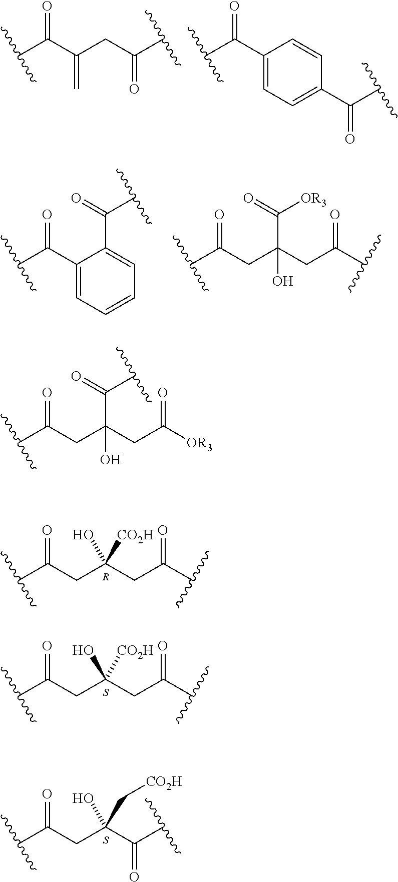

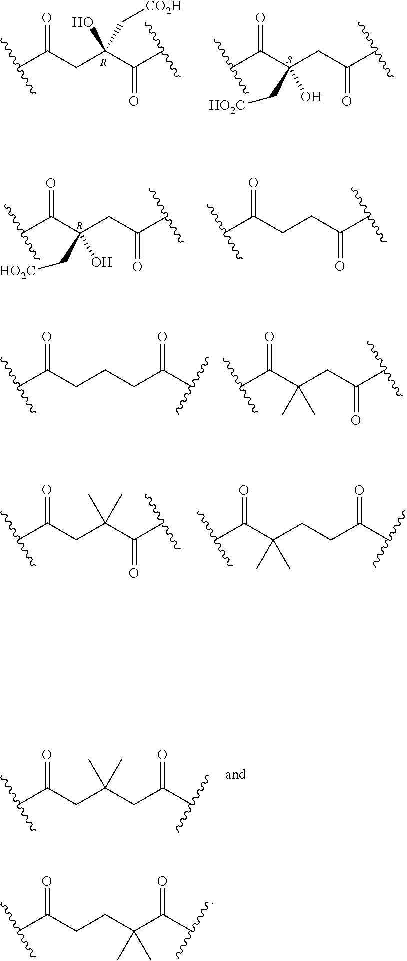

19. The prodrug of claim 1, wherein —R is an amino acid residue containing from 2 to 20 carbon atoms or a peptide formed from 2 to 10 independently selected amino acids each containing from 2 to 20 carbon atoms and the amino acid residue or peptide (—R) terminates with a carboxylic acid group —COOH or an amino group —NH₂.

20. The prodrug of claim 1, wherein —R is an amino acid residue containing from 2 to 20 carbon atoms or a peptide formed from 2 to 10 independently selected amino acids each containing from 2 to 20 carbon atoms and the amino acid residue or peptide (—R) terminates with a carboxylate ester COOR^{a}.

21. The prodrug of claim 1, wherein —R is an amino amide residue containing from 2 to 10 carbon atoms and terminating with a —CONR^{a}R^{b} group.

22. The prodrug of claim 1, wherein R is —OR^{a}.

23. The prodrug of claim 1, wherein R is —NR^{a}R^{b}.

24. The prodrug of claim 21, wherein R^{b} is selected from the group consisting of: H, Me, Et and cyclopropyl.

25. The prodrug of claim 20, wherein R^{a} is selected from the group consisting of: H, Me, Et and cyclopropyl.

26. (canceled)

27. A pharmaceutical composition comprising a prodrug of of claim 5 or a pharmaceutically acceptable salt thereof and a pharmaceutically acceptable excipient.

28. The prodrug of claim 24, wherein R^{b} is H.

29. The prodrug of claim 25, wherein R^{a} is H.
